SEQUENCE LISTING (8 sequences)

SEQ ID NO: 1 - DNA, 36 bp, Artificial Sequence
  Synthetic polynucleotide
  misc_feature (21)..(21): Cy5
  misc_feature (36)..(36): SH
ccctaaccctaaccctaacccttttttttttttttt

SEQ ID NO: 2 - DNA, 36 bp, Artificial Sequence
  Synthetic polynucleotide
  misc_feature (21)..(21): CY5
  misc_feature (36)..(36): SH
tttctatcgcgtacaatctgcttttttttttttttt

SEQ ID NO: 3 - DNA, 19 bp, Artificial Sequence
  Synthetic polynucleotide
  misc_feature (1)..(1): CY3
aaaaaaagggttagggtta

SEQ ID NO: 4 - DNA, 19 bp, Artificial Sequence
  Synthetic polynucleotide
  misc_feature (1)..(1): CY3
aaaaaaagcagattgtacg

SEQ ID NO: 5 - DNA, 40 bp, Artificial Sequence
  Synthetic polynucleotide
  misc_feature (1)..(1): DBCO- TEG
tttttttttttttttcccctaacdcctaacccctaactcc

SEQ ID NO: 6 - DNA, 40 bp, Artificial Sequence
  Synthetic polynucleotide
  misc_feature (1)..(1): DBCO-TEG
tttttttttttttttttttttttdtttttttttttttttt

SEQ ID NO: 7 - DNA, 13 bp, Artificial Sequence
  Synthetic polynucleotide
  misc_feature: DBCO TEG
ttttttttttttt

SEQ ID NO: 8 - DNA, 16 bp, Artificial Sequence
  Synthetic polynucleotide
cccccdcccccccccc

(19) United States
(12) Patent Application Publication
(10) Pub. No.: US 2023/0088835 A1
(43) Pub. Date: Mar. 23, 2023

(54) FIT-FLARES FOR DETECTION OF INTRACELLULAR ANALYTES IN LIVE CELLS

(71) Applicant: NORTHWESTERN UNIVERSITY, Evanston, IL (US)

(72) Inventors: Chad A. Mirkin, Wilmette, IL (US); Sasha B. Ebrahimi, DeKalb, IL (US); Devleena Samanta, Evanston, IL (US)

(21) Appl. No.: 17/908,809

(22) PCT Filed: Jul. 20, 2020
(86) PCT No.: PCT/US20/42835

§ 371 Date: Sep. 1, 2022

Related U.S. Application Data
(60) Provisional application No. 62/984,138, filed on Mar. 2, 2020.

(51) Int. Cl. G01N 33/53; G01N 33/535

(52) U.S. Cl. CPC G01N 33/5308; G01N 33/535

(57) ABSTRACT

The present disclosure is directed to spherical nucleic acids (SNAs) comprising a nanoparticle core and an oligonucleotide, use of the SNAs to, e.g., detect target analytes, and methods of making the SNAs. In various embodiments, the target analyte is detected using the nanoparticle core, the oligonucleotide, or both. In some embodiments, the oligonucleotide comprises a detectable marker situated at an internal location within the oligonucleotide. In some aspects, the disclosure provides methods for detecting a target analyte comprising the step of contacting the target analyte with a spherical nucleic acid (SNA) and an agent, the SNA comprising a protein core and an oligonucleotide attached thereto, wherein the contacting of the protein core with the target analyte results in a change in the target analyte that is detectable by the agent, thereby detecting the target analyte.

### CROSS-REFERENCE TO RELATED APPLICATIONS

This application claims the priority benefit under 35 U.S.C. § 119(e) of U.S. Provisional Patent Application No. 62/984,138, filed Mar. 2, 2020, which is incorporated herein by reference in its entirety.

### STATEMENT OF GOVERNMENT INTEREST

This invention was made with government support under U54CA199091 awarded by the National Institutes of Health, FA8650-15-2-5518 awarded by The Air Force Research Laboratory, FA9550-17-1-0348 awarded by The Air Force Office of Scientific Research, DE-S00000989 awarded by The Department of Energy, and N00014-15-1-0043 awarded by the Office of Naval Research. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2020-044A_Seqlisting.txt”, which was created on Jul. 20, 2020 and is 2,385 bytes in size. The subject matter of the Sequence Listing is incorporated herein in its entirety by reference.

### BACKGROUND

The chemical analysis of live cells at the molecular level provides fundamental insight into dynamic cellular processes, informs about the role of intracellular analytes in disease progression, and has guided the development of new medical diagnostic tools.¹⁻⁶ Although fluorescent probes based on both molecular recognition (binding-based sensing)^{3,7} and molecular reactivity (activity-based sensing)⁸ have led to significant new capabilities, the majority of techniques either necessitate the fixing or lysis of the cells, the use of cytotoxic transfection reagents, or the genetic encoding of the cells. Specifically, protein- and nucleic acid-based approaches, such as enzyme-linked immunosorbent assays,⁹ genetically encoded-fluorescent proteins¹⁰ and RNA sensors,¹¹ polymerase chain reaction,¹² and fluorescence in situ hybridization,¹³ are routinely used to detect a wide variety of biological analytes. However, exogenous proteins and nucleic acids are not efficiently internalized by cells.

The first examples of spherical nucleic acid (SNA)-based intracellular probes were NanoFlares (NFs).^{17,18} The NF construct generally includes a gold nanoparticle core that acts as a quencher. Oligonucleotide duplexes comprising a recognition strand and a shorter fluorophore-labeled reporter strand are immobilized onto the gold nanoparticle through a gold-thiol linkage. Inside the cell, the target of interest displaces the reporter strand as it binds to the recognition sequence, and results in fluorescence turn on due to separation of the fluorophore and quencher. By designing the recognition strand to be complementary to nucleic acids in cells, genetic content can be measured.¹⁷⁻²² On the other hand, using aptamer and DNAzyme sequences, ions, small molecules, and proteins can be detected.²³⁻²⁵ NFs allow for live-cell genetic and metabolic analyses,^{17,23} the sorting and isolation of circulating tumor cells based on variations in genetic profiles,²⁶ and the identification of diseased tissue in vivo.²⁷⁻²⁹

### SUMMARY

A versatile platform for studying many different types of analytes in live cells remains an outstanding challenge. Accordingly, in some aspects, the disclosure provides constructs referred to as FIT-Flares, comprising a nanoparticle core functionalized with one or more oligonucleotides, that are capable of studying analytes in single living cells with subcellular resolution. See FIGS. 34 and 35. The oligonucleotide shell allows FIT-Flares to be actively taken up by cells without the need of transfection reagents. It is shown herein that both the oligonucleotide and the nanoparticle core can be used as sensor elements. By way of example, by choosing the oligonucleotide sequence to be a FIT-aptamer, the molecule or ions to which the aptamer binds can be detected. Choosing a “functional core” (e.g., proteins, polymers, or liposomes) dramatically expands the capabilities of FIT-Flares by allowing amplified detection through enzymatic assays, by enabling multiplexed detection, and by facilitating the development of theranostic platforms. Importantly, by labeling the nanoparticle core with a normalizing dye, quantitative analyte measurements can be made. Taken together, FIT-Flares constitute the only nanoparticle-based platform capable of subcellular resolution and single-cell measurements of analytes in living cells without the need for transfection reagents.

Applications of the technology described herein include, but are not limited to:
Detecting and imaging analytes in living cells and complex milieu
Quantifying levels of analytes in cells and complex milieu
Quantifying levels of analytes in cells at subcellular resolution with organelle-level specificity
Imaging analytes with spatiotemporal resolution in living cells
Regulating and detecting analytes of interest (theranostic)
Delivery of proteins that can act as both a therapeutic and sensor component (theranostic)

Advantages of the technology disclosed herein include, but are not limited to:
Previously disclosed constructs represented the first transfection reagent-free probes for monitoring analytes in live cells. Such constructs are described in, e.g., U.S. Pat. Nos. 8,507,200, 9,890,427, and 10,301,622, each of which is incorporated herein by reference in its entirety.
The use of a FIT-based strategy as described herein makes FIT-Flares resistant to false positive signals as this platform is quencher-free.
FIT-Flares enter cells actively, overcoming limitations associated with delivery of payload into cells. In particular, a major challenge in the biological imaging field is the effective delivery of both proteins and DNA into living cells, which is made possible by the FIT-Flare platform.
FIT Flares represent a new platform that allows FIT-based recognition elements to be delivered into live cells without the use of transfection reagents.
In the context of a NanoFlare, a FIT-based strategy eliminates the use of a “flare” strand. This has important consequences in intracellular detection contexts. The presence of a flare strand partially blocks the binding site in the recognition sequence, reducing binding affinity and sensitivity, and retarding kinetics of sensing. The new design disclosed herein allows the detection of analytes with higher sensitivity as well as analytes that fluctuate on faster timescales.
The FIT-based oligonucleotide probes contemplated herein bind to the target and fluoresces, which allows spatial monitoring of the analyte.
FIT-Flares can be modified with targeting moieties to enter specific cells in a complex mixture of cells or enter cells through different pathways. This would allow the organelle-specific analyte mapping, which has been a challenge in the field of detection.
Using cores that can encapsulate different molecules (e.g., liposomes and PLGA), one can deliver dyes for detecting analytes in intracellular compartments (detection and diagnostics) or one can deliver molecules (small molecules or proteins) for theranostic purposes.
Using protein cores, one can deliver proteins into cells (proteins do not readily enter cells). If an enzyme is used as the core, one can detect additional intracellular analytes based on enzymatic assays in live cells. If a fluorescent protein is used, one can detect intracellular analytes based on fluorescence enhancement in the presence of the analyte to which the protein binds. Any protein that has been used as a genetically encoded sensor can be delivered to cells exogenously to detect intracellular analytes. This is particularly important for detecting analytes in clinical samples where cells cannot be genetically engineered to express fluorescent probes.

Accordingly, in some aspects the disclosure provides SNAs comprising a protein core and one or more oligonucleotides attached thereto. As described herein, various protein cores are contemplated and include, without limitation, an enzyme, a therapeutic protein, a structural protein (e.g., actin), a defensive protein (e.g., an antibody), a storage protein (e.g., ovalbumin), a transport protein (e.g., hemoglobin), a hormone (e.g., insulin), a receptor protein (e.g., G-Protein Coupled Receptors), a motor protein (e.g., kinesin, dynein, or myosin), or a fluorescent protein. Selection of a particular protein core is based on the particular desired application. For example and without limitation, if an enzyme is used as the core then a target analyte can be detected based on enzymatic assays. In addition, the one or more oligonucleotides attached to the protein core provide additional capabilities including cell uptake, gene inhibition, immunostimulatory, and/or detection capabilities, each as described herein.

In various aspects, the present disclosure provides a class of probes, called FIT-Flares, capable of multiplexed detection of analytes in living cells at single cell resolution. FIT-Flares comprise FIT-based recognition oligonucleotides conjugated to a nanoparticle core. The FIT-based recognition oligonucleotides can in principle be designed to detect any molecule or ion of interest. Furthermore, any nanoparticle core can be used, allowing for the core to, for example, act as a sensor element or act as an agent that can encapsulate dyes capable of imaging processes inside of cells. Taken together, FIT-Flares are a multifunctional platform that represents the only nanoparticle-based method to make quantitative measurements within live cells at subcellular and single-cell resolution.

Studying intracellular analytes in living cells taken from clinical samples is an outstanding challenge. FIT-Flares constitute probes that are, in some aspects, used as simultaneous transfection and imaging agents in these cells, giving information about cellular analyte levels with high sensitivity and lack of false-positive signal.

Accordingly, in some aspects the disclosure provides a method for detecting a target analyte comprising the step of contacting the target analyte with a spherical nucleic acid (SNA), the SNA comprising a nanoparticle core and an oligonucleotide attached thereto, wherein the oligonucleotide comprises a detectable marker situated at an internal location within the oligonucleotide, wherein the contacting results in binding of the target analyte to the oligonucleotide, and wherein the binding results in restriction of internal rotation of the marker, resulting in a detectable change and thereby detecting the target analyte. In some embodiments, a plurality of the oligonucleotides is attached thereto. In some embodiments, the detectable change is an increase in fluorescence. In some embodiments, the oligonucleotide is an aptamer. In some embodiments, target analyte binding to the oligonucleotide results in forced intercalation (FIT) of the marker between base pairs of the oligonucleotide. In some embodiments, the detectable marker is a marker with internal rotation-dependent fluorescence. In some embodiments, the detectable marker is a viscosity-sensitive marker. In some embodiments, the detectable marker is thiazole orange (TO), quinoline blue, quinoline violet, thiazole red, a derivative thereof, or a cyanine derivative. In some embodiments, the detectable change is proportional to concentration of the target analyte. In some embodiments, the target analyte is a protein, an ion, a small molecule, a lipid, a carbohydrate, an oligosaccharide, a cell, a nucleic acid, or a combination thereof. In some embodiments, the ion is a metal ion. In some embodiments, the metal ion is a mercury ion, a copper ion, a silver ion, a zinc ion, a gold ion, a manganese ion, or a combination thereof. In some embodiments, the ion is a hydrogen ion. In some embodiments, the detectable change is indicative of a pH change. In some embodiments, the oligonucleotide is DNA, RNA, or a modified form thereof. In some embodiments, the oligonucleotide is about 5 to about 1000 nucleotides in length. In some embodiments, the oligonucleotide is about 10 to about 100 nucleotides in length. In some embodiments, the oligonucleotide comprises a spacer. In some embodiments, the detectable marker is situated at a position that is x nucleotides from a terminus of the oligonucleotide, wherein x is an integer that is 1, n/2, or any integer between 1 and n/2, wherein n is (i) the length of the oligonucleotide and (ii) an even number. In some embodiments, the detectable marker is situated at a position that is x nucleotides from a terminus of the oligonucleotide, wherein x is an integer that is 1, (n+1)/2, or any integer between 1 and (n+1)/2, wherein n is (i) the length of the oligonucleotide and (ii) an odd number. In some embodiments, the detectable marker is situated at a position that is 1, 2, 3, 4, 5, 6, 7, 8, 9, or 10 nucleotides from a terminus of the oligonucleotide. In some embodiments, the SNA further comprises an inhibitory oligonucleotide attached thereto. In some embodiments, the inhibitory oligonucleotide is antisense DNA, small interfering RNA (siRNA), an aptamer, a short hairpin RNA (shRNA), a DNAzyme, or an aptazyme. In some embodiments, the SNA further comprises an immunostimulatory oligonucleotide attached thereto. In some embodiments, the immunostimulatory oligonucleotide is a toll-like receptor (TLR) agonist. In further embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the SNA further comprises a toll-like receptor (TLR) antagonist. In further embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some aspects, the disclosure provides a method for identifying a nucleotide recognition sequence that is useful to detect a target analyte comprising the steps of contacting the target analyte with a spherical nucleic acid (SNA), the SNA comprising a nanoparticle core and an aptamer attached thereto, wherein the aptamer comprises a candidate nucleotide sequence and a detectable marker situated at an internal location within the aptamer, wherein binding of the candidate nucleotide sequence to the target analyte results in an increase in fluorescence due to restriction of internal rotation of the detectable marker; comparing fluorescence before and after the contacting, and identifying the candidate nucleotide sequence as the nucleotide recognition sequence from an increase in fluorescence after the contacting. In some embodiments, target analyte binding to the aptamer results in forced intercalation (FIT) of the marker between oligonucleotide base pairs of the aptamer. In some embodiments, a plurality of the aptamers are attached thereto. In some aspects, the disclosure provides a method for detecting a target analyte comprising the step of contacting the target analyte with a spherical nucleic acid (SNA), the SNA comprising a nanoparticle core and a plurality of oligonucleotides attached thereto, the plurality of oligonucleotides comprising (a) an aptamer or portion thereof comprising (i) nucleotide sequence X, (ii) nucleotide sequence Y which binds to the target analyte, either alone or in combination with nucleotide sequence Y′, and (iii) a detectable marker situated at an internal location within the aptamer, and (b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y, wherein the contacting results in hybridization of nucleotide sequence X with nucleotide sequence X′ and binding of the target analyte with nucleotide sequence Y and nucleotide sequence Y′, wherein the binding of nucleotide sequence X with nucleotide sequence X′ and the target analyte with nucleotide sequence Y and nucleotide sequence Y′ result in restriction of internal rotation of the marker, resulting in a detectable change and thereby detecting the target analyte. In some embodiments, nucleotide sequence Y and nucleotide sequence Y′ bind to different binding sites of the target analyte. In some embodiments, nucleotide sequence Y and nucleotide sequence Y′ together bind to the same binding site of the target analyte. In some embodiments, the binding of nucleotide sequence X with nucleotide sequence X′ and the target analyte with nucleotide sequence Y and nucleotide sequence Y′ result in forced intercalation (FIT) of the marker between oligonucleotide base pairs of the aptamer and the additional aptamer. In some embodiments, the detectable marker is situated at a position that is x nucleotides from a terminus of the aptamer, wherein x is an integer that is 1, n/2, or any integer between 1 and n/2, wherein n is (i) the length of the aptamer and (ii) an even number. In some embodiments, the detectable marker is situated at a position that is x nucleotides from a terminus of the aptamer, wherein x is an integer that is 1, (n+1)/2, or any integer between 1 and (n+1)/2, wherein n is (i) the length of the aptamer and (ii) an odd number. In some embodiments, the detectable marker is situated at a position that is 1, 2, 3, 4, 5, 6, 7, 8, 9, or 10 nucleotides from a terminus of the aptamer. In some embodiments, the plurality of oligonucleotides comprises an inhibitory oligonucleotide. In further embodiments, the inhibitory oligonucleotide is antisense DNA, small interfering RNA (siRNA), an aptamer, a short hairpin RNA (shRNA), a DNAzyme, or an aptazyme. In some embodiments, the plurality of oligonucleotides comprises an immunostimulatory oligonucleotide. In some embodiments, the immunostimulatory oligonucleotide is a toll-like receptor (TLR) agonist. In further embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the plurality of oligonucleotides comprises a toll-like receptor (TLR) antagonist. In further embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some embodiments, the nanoparticle core is a metallic core, a semiconductor core, an insulator core, an upconverting core, a micellar core, a dendrimer core, a liposomal core, a polymer core, a metal-organic framework core, a protein core, or a combination thereof. In some embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), or chitosan. In some embodiments, the polymer is poly(lactic-co-glycolic acid) (PLGA). In some embodiments, the nanoparticle core is gold, silver, platinum, aluminum, palladium, copper, cobalt, indium, cadmium selenide, iron oxide, fullerene, metal-organic framework, zinc sulfide, or nickel. In some embodiments, the nanoparticle core is a protein core. In some embodiments, contacting the protein core with the target analyte results in an additional detectable change. In some embodiments, the additional detectable change is a fluorescence change or a luminescence change. In some embodiments, the protein core is an enzyme, a therapeutic protein, a structural protein, a defensive protein, a storage protein, a transport protein, a hormone, a receptor protein, a motor protein, or a fluorescent protein. In some embodiments, the protein core comprises an enzyme that interacts with and allows detection of an additional target analyte. In some embodiments, the target analyte and the additional target analyte are the same. In some embodiments, the enzyme is glucose oxidase (GOx), cholesterol oxidase, luciferase, or creatinine deaminase. In some embodiments, the methods further comprise contacting the additional target analyte with an agent. In some embodiments, the agent is associated with the external side of the nanoparticle core. In some embodiments, the agent is encapsulated in the nanoparticle core. In some embodiments, the agent is associated with the oligonucleotide. In some embodiments, the agent is added exogenously. In some embodiments, the additional target analyte is detectable after contacting the additional target analyte with the agent. In some embodiments, the agent is a small molecule. In some embodiments, the small molecule is a dye or a luminophore. In some embodiments, the dye is a normalizing dye, or a dye that localizes to an organelle.

In some aspects, the disclosure provides a method for detecting a target analyte comprising the step of contacting the target analyte with a spherical nucleic acid (SNA) and an agent, the SNA comprising a protein core and an oligonucleotide attached thereto, wherein the contacting of the protein core with the target analyte results in a change in the target analyte that is detectable by the agent, thereby detecting the target analyte. In some embodiments, the SNA comprises a plurality of oligonucleotides attached thereto. In some embodiments, the change that is detectable by the agent is a fluorescence change or a luminescence change. In some embodiments, the change that is detectable by the agent is proportional to concentration of the target analyte. In some embodiments, the protein core comprises an enzyme. In some embodiments, the enzyme is glucose oxidase (GOx), cholesterol oxidase, luciferase, or creatinine deaminase. In some embodiments, the agent is associated with the external side of the nanoparticle core. In some embodiments, the agent is encapsulated in the nanoparticle core. In some embodiments, the agent is associated with the oligonucleotide. In some embodiments, the agent is added exogenously. In some embodiments, the agent is a small molecule. In some embodiments, the small molecule is a dye or a luminophore. In some embodiments, the dye is a normalizing dye or a dye that localizes to an organelle. In some embodiments, the oligonucleotide is an inhibitory oligonucleotide. In some embodiments, the inhibitory oligonucleotide is antisense DNA, small interfering RNA (siRNA), an aptamer, a short hairpin RNA (shRNA), a DNAzyme, or an aptazyme. In some embodiments, the oligonucleotide is an immunostimulatory oligonucleotide. In some embodiments, the immunostimulatory oligonucleotide is a toll-like receptor (TLR) agonist. In further embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the oligonucleotide is a toll-like receptor (TLR) antagonist. In further embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some embodiments, the target analyte is a protein, an ion, a small molecule, a lipid, a carbohydrate, an oligosaccharide, a cell, or a combination thereof. In some embodiments, the target analyte is not a nucleic acid. In some embodiments, the ion is a metal ion. In further embodiments, the metal ion is a mercury ion, a copper ion, a silver ion, zinc ion, gold ion, manganese ion, or a combination thereof. In some embodiments, the ion is a hydrogen ion. In some embodiments, the detectable change is indicative of a pH change. In some embodiments, the SNA further comprises a therapeutic agent. In some embodiments, the therapeutic agent is associated with the nanoparticle core. In some embodiments, the therapeutic agent is encapsulated in the nanoparticle core or is attached to the external side of the nanoparticle core. In some embodiments, the therapeutic agent is associated with the oligonucleotide. In some embodiments, the target analyte is detected intracellularly.

### BRIEF DESCRIPTION OF THE DRAWINGS

FIG. 1 shows the ¹H NMR (400 MHz, methanol-d₄, 298 K) spectrum of 3-methyl-2-(methylthio)-benzothiazolium tosylate.

FIG. 2 shows the ¹³C NMR (101 MHz, methanol-d₄, 298 K) spectrum of 3-methyl-2-(methylthio)-benzothiazolium tosylate.

FIG. 3 shows the ¹H NMR (400 MHz, methanol-d₄, 298 K) spectrum of carboxymethylated thiazole orange.

FIG. 4 shows results of a fluorescence melt experiment to determine the melting temperature of pH-sensitive i-motif and pH-insensitive control NFs recognition/flare duplex. It is seen from the plot that the melting temperatures are nearly identical. Data points and error bars represent the mean and standard deviation of three replicates, respectively.

FIG. 5 shows fluorescence enhancement (I_{f}/I₀) versus pH for gold NF constructs in buffer. Fluorescence enhancement is defined as the fluorescence intensity of the solution at a given pH (I_{f}) relative to the lowest fluorescence intensity of the solution across all the pH tested (I₀). All fluorescence intensities are corrected for the fluorescence intensity of the buffer. Data points and error bars represent the mean and standard deviation of three replicates, respectively.

FIG. 6 shows fluorescence enhancement over time of 100 μL of 2 nM i-motif or control gold NF in the presence of 10 μL of 0.2 U/μL DNAse I. Fluorescence enhancement is defined as the fluorescence intensity of the solution at a given time (I_{f}) relative to the initial fluorescence intensity of the same solution (I₀). All fluorescence intensities are corrected for the fluorescence intensity of the buffer. In the presence of DNAse I, the fluorescence increased by over 15-fold while in the absence of DNAse I, the fluorescence remains unchanged. Data points and error bars represent the mean and standard deviation of three replicates, respectively.

FIG. 7 shows fluorescence enhancement over time of MDA-MB-231 cells treated with control gold NFs. Fluorescence enhancement is defined as the mean fluorescence intensity of the cells at a given time (I_{f}) relative to the mean fluorescence intensity (I₀) at the initial timepoint. All fluorescence intensities are corrected for the mean fluorescence intensity of untreated cells. Time 1 h corresponds to a 1 h pulse, 0 h chase. Time 3 h corresponds to a 1 h pulse, 2 h chase. Time 5 h corresponds to 1 h pulse, 4 h chase. The fluorescence increased by approximately 3.5 fold over time. Data points and error bars represent the mean and standard deviation of three replicates, respectively.

FIG. 8 shows fluorescence enhancement over time of 100 μL of 500 nM (by DNA) of ProTOn or control ProSNA in the presence of 10 μL of 0.2 U/μL DNAse 1. The fluorescence of samples without DNAse I was monitored over time as a control. Fluorescence enhancement is defined as the fluorescence intensity at a given time (I_{f}) relative to the fluorescence intensity (I₀) at the initial timepoint. All fluorescence intensities are corrected for the fluorescence intensity of the buffer. Data points and error bars represent the mean and standard deviation of three replicates, respectively.

FIG. 9 shows fluorescence enhancement over time of MDA-MB-231 cells treated with control ProSNAs. Fluorescence enhancement is defined as the mean fluorescence intensity of the cells at a given time (I_{f}) relative to the mean fluorescence intensity (I₀) at the initial timepoint. All fluorescence intensities are corrected for the mean fluorescence intensity of untreated cells. Time 1 h corresponds to a 1 h pulse, 0 h chase. Time 3 h corresponds to a 1 h pulse, 2 h chase. Time 5 h corresponds to a 1 h pulse, 4 h chase. Unlike with gold NFs (FIG. 7), the fluorescence does not significantly change over time. Data points and error bars represent the mean and standard deviation of three replicates, respectively.

FIG. 10 shows fluorescence enhancement of pHrodo™ Red in (A) buffer at different pH (500 nM pHrodo, excitation: 560 nm, emission: 610 nm) and (B) cells clamped at different pH. Fluorescence enhancement is defined as the fluorescence intensity of the pHrodo™ Red solution/cells at a given pH (I_{f}) relative to the fluorescence intensity (I₀) at pH 7.5. All fluorescence intensities are corrected for the fluorescence intensity of the buffer/untreated cells. Intracellularly, pHrodo™ Red results in an approximately 2-fold fluorescence enhancement when clamped at pH 5.5 relative to pH 7.5. Data points and error bars represent the mean and standard deviation of three replicates, respectively.

FIG. 11 shows SDS-PAGE gel of β-gal and β-gal ProSNA treated with trypsin (protease) shows that while β-gal degrades over a time course of 70 min (as evidenced by the appearance of multiple bands at lower molecular weights), β-gal ProSNA does not. The β-gal ProSNA used in this specific study consists of the DNA sequence 5′-DBCO-dT-(sp18)₂T₃₀-3′.

FIG. 12 shows UV-vis characterization of GOx-SNAs. The absorbance was normalized relative to the GOx peak at 280 nm which is set to a value of 1.

FIG. 13 shows a comparison of catalytic activity of GOx and GOx-SNAs at 37° C. 20 nM GOx-SNA or 20 nM native GOx are treated with 0 mM or 1 mM glucose in 1×PBS containing 5 μM FBBBE. The fluorescence at each time point was corrected for the fluorescence of the dye alone in buffer. The fluorescence was monitored by exciting FBBBE at 485 nm and collecting the emission at 528 nm. The results showed that the protein's native activity was retained in the SNA form. Data points and error bars represent the mean and standard deviation of three replicates, respectively.

FIG. 14 shows fluorescence enhancement of 20 nM GOx-SNAs+5 μM FBBBE in the presence of varying amounts of glucose at 37° C. The fluorescence enhancement is calculated as the ratio, I_{c,30}/I_{0,30}. I_{c,30} represents the fluorescence of the solution at the 30 min time point when a concentration c of glucose is added. I_{0,30} represents the fluorescence of the solution at the 30 min time point when a concentration of 0 mM of glucose is added. The fluorescence is corrected for the fluorescence of the buffer. The fluorescence is monitored by exciting FBBBE at 460 nm and collecting the emission at 530 nm. Data points and error bars represent the mean and standard deviation of three replicates, respectively.

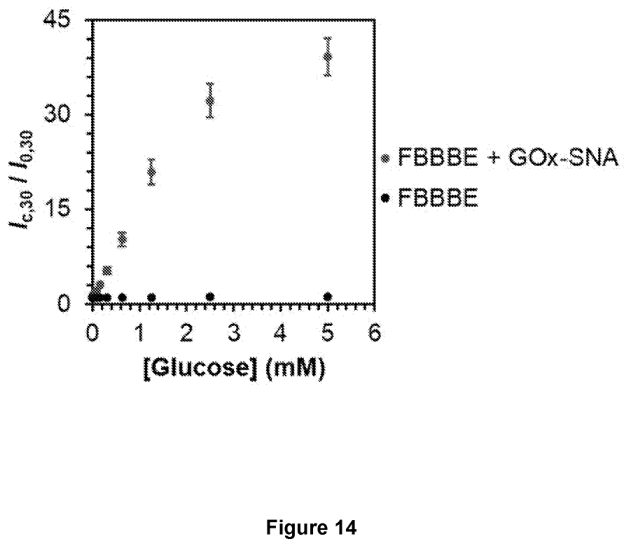

FIG. 15 shows fluorescence enhancement over time of AF-647 conjugated to GOx-SNAs in the presence of varying amounts of glucose and 5 μM FBBBE 1×PBS at 37° C. The fluorescence enhancement is calculated as the ratio of I_{c,t}/I_{0,0}. I_{c,t} represents the fluorescence of the solution at the time t when a concentration c of glucose is added. I_{0,0} represents the fluorescence of the solution at the initial time point when a concentration of 0 mM of glucose is added. These results show that the AF-647 signal from GOx-SNAs is not affected by the formation of FBBBE. The slight decrease in fluorescence over time is attributed to photobleaching. The fluorescence is monitored by exciting GOx-SNAs at 640 nm and collecting the emission at 700 nm. Data points and error bars represent the mean and standard deviation of three replicates, respectively.

FIG. 16 shows the selectivity of GOx-SNAs for glucose over other sugars in a complex mixture. 10 nM GOx-SNAs were incubated with 5 μM FBBBE at 37° C. for 30 min in the presence or absence of 5 mM of sugars. The fluorescence observed when 5 mM glucose was added to the GOx-SNA/FBBBE solution was normalized to a value of one. The other values are plotted relative to this value. The fluorescence of all three data points was corrected for the fluorescence of a solution of containing only FBBBE. The fluorescence was monitored by exciting FBBBE at 485 nm and collecting the emission at 528 nm. Data points and error bars represent the mean and standard deviation of three replicates, respectively.

FIG. 17 depicts an example flow cytometry gating strategy. (A) Cells were first distinguished from debris based on forward and side scatter. (B) Single cells were distinguished from clusters of cells. (C) Live cells were selected based on the fluorescence of the dye DAPI which preferentially stains dead cells. (D) The fluorescence histogram of live cells in the FBBBE channel was plotted. In this example, EG7-OVA cells treated with FBBBE were analyzed.

FIG. 18 shows glucose detection in MDA-MB-231 cells. Representative fluorescence histograms of untreated cells, cells treated with 50 μM FBBBE, and cells treated with 40 nM GOx-SNA and 50 μM FBBBE.

FIG. 19 shows glucose detection in U87 cells. Representative fluorescence histograms of untreated cells, cells treated with 50 μM FBBBE, and cells treated with 40 nM GOx-SNA and 50 μM FBBBE.

FIG. 20 shows glucose detection in SKOV-3 cells. Representative fluorescence histograms of untreated cells, cells treated with 50 μM FBBBE, and cells treated with 40 nM GOx-SNA and 50 μM FBBBE.

FIG. 21 shows glucose detection in EL4 cells. Representative fluorescence histograms of untreated cells, cells treated with 50 μM FBBBE, and cells treated with 40 nM GOx-SNA and 50 μM FBBBE.

FIG. 22 shows glucose detection in Human Dermal Fibroblast (HDF) cells. Representative fluorescence histograms of untreated cells, cells treated with 50 μM FBBBE, and cells treated with 40 nM GOx-SNA and 50 μM FBBBE.

FIG. 23 shows glucose detection in MC38 cells. Representative fluorescence histograms of untreated cells, cells treated with 50 μM FBBBE, and cells treated with 40 nM GOx-SNA and 50 μM FBBBE.

FIG. 24 shows glucose detection in NIH-3T3 cells. Representative fluorescence histograms of untreated cells, cells treated with 50 μM FBBBE, and cells treated with 40 nM GOx-SNA and 50 μM FBBBE.

FIG. 25 shows glucose detection in 4T1 cells. Representative fluorescence histograms of untreated cells, cells treated with 50 μM FBBBE, and cells treated with 40 nM GOx-SNA and 50 μM FBBBE.

FIG. 26 shows glucose detection in EG7-OVA cells. Representative fluorescence histograms of untreated cells, cells treated with 50 μM FBBBE, and cells treated with 40 nM GOx-SNA and 50 μM FBBBE.

FIG. 27 shows the intracellular response of GOx-SNAs to varying glucose concentrations in cell culture media. (A) Representative fluorescence histograms of GOx-SNA-treated cells incubated in cell culture media containing 0 and 25 mM glucose. (B) Mean fluorescence of GOx-SNA-treated cells incubated in cell culture media containing different glucose concentrations (I_{f}) relative to the mean fluorescence of cells incubated in 0 mM glucose-containing media (I₀). Data points and error bars represent the mean and standard deviation of three replicates, respectively. (C) Relative fluorescence values of cells incubated in cell culture media containing 0 and 25 mM glucose as measured using cell lysates through a commercially available glucose assay kit. Data points and error bars represent the mean and standard deviation of three replicates, respectively.

FIG. 28 shows the intracellular response of GOx-SNAs to increase of glucose uptake. (A) Representative fluorescence histograms of GOx-SNA-treated cells incubated in cell culture media containing 0 and 100 nM insulin. (B) Mean fluorescence of GOx-SNA-treated cells incubated in cell culture media containing different insulin concentrations (I_{f}) relative to the mean fluorescence of cells incubated in 0 nM insulin-containing media (I₀). Data points and error bars represent the mean and standard deviation of two replicates, respectively.

FIG. 29 shows the intracellular response of GOx-SNAs to inhibition of glucose uptake. (A) Representative fluorescence histograms of GOx-SNA-treated cells incubated in cell culture media containing 0 and 10 μM cytochalasin B. (B) Mean fluorescence of GOx-SNA-treated cells incubated in cell culture media containing different cytochalasin B concentrations (I_{f}) relative to the mean fluorescence of cells incubated in 0 μM cytochalasin B-containing media (I₀). Data points and error bars represent the mean and standard deviation of three replicates, respectively. (C) Relative fluorescence values of cells incubated in cell culture media containing 0 and 10 μM cytochalasin B as measured using cell lysates through a commercially available glucose assay kit. Data points and error bars represent the mean and standard deviation of three replicates, respectively.

FIG. 30 shows (A) Structure of β-galactosidase with lysine and cysteine residues highlighted. (B) Structure of the forced intercalation dye thiazole orange, TO (carboxymethylated derivative). (C) Unfolded i-motif sequence with a single base replaced with TO. (D) Folded i-motif with TO intercalated between base pairs. (E) Structure of ProTOn at pH 7.5 and pH 5.5. The formation of the i-motif structure leads to fluorescence turn on of TO.

FIG. 31 shows (A) In vitro fluorescence response of ProTOn and a control probe as a function of pH. The fluorescence of TO increased as pH decreases due to the formation of i-motifs in ProTOn. The control probe did not form an i-motif and, therefore, shows no change in fluorescence. The fluorescence of AF-647 remained unchanged for both ProTOn and the control probe. (B) TO channel fluorescence response of MDA-MB-231 cells treated with ProTOn and a control probe. ProTOn-treated cells clamped at pH 5.5 are almost twice as fluorescent as those clamped at pH 7.5. Cells treated with the control probe showed no significant difference in fluorescence.

FIG. 32 shows (A) Structure of glucose oxidase SNAs (GOx-SNAs). (B) Structure of fluorescein bis (benzyl boronic ester), FBBBE. (C) A two-step assay developed for glucose detection. (i) First, GOx-SNAs catalyze the conversion of β-D-glucose to D-glucono-1,5-lactone with the formation of H₂O₂. (ii) The H₂O₂ formed reacts with non-fluorescent FBBBE and yields highly fluorescent fluorescein.

FIG. 33 shows (A) In vitro fluorescence response of GOx-SNA to increasing concentrations of glucose. The y-axis shows the observed fluorescence (I_{c,t}) for a particular concentration, c, of glucose at time, t, relative to the fluorescence (I_{0,0}) of GOx-SNA at the initial timepoint in the absence of glucose. Over 120-fold fluorescence enhancement is observed in the presence of glucose. (B) In vitro selectivity of GOx-SNA against other sugars. (C) Fluorescence of EL4 cells under different treatment conditions. Cells treated with GOx-SNAs and FBBBE fluoresced approximately 12-fold more compared to cells treated with FBBBE alone. (D) Representative fluorescence histograms corresponding to data in (C).

FIG. 34 depicts an exemplary design of a FIT-Flare highlighting its components and their significance to the detection scheme. Specifically, this scheme shows an example of a liposomal spherical nucleic acid-based intracellular probe. The nucleic acid shell facilitates cellular uptake and can detect analytes using FIT-based probes as described herein. The liposomal core can be used to encapsulate additional molecules such as therapeutic agents for the development of theranostics, or fluorescent dyes. Importantly, the fluorescent dye could be a normalizing dye that allows ratiometric analysis or an additional probe that can enable the simultaneous measurement of multiple analytes in the same compartment as the DNA.

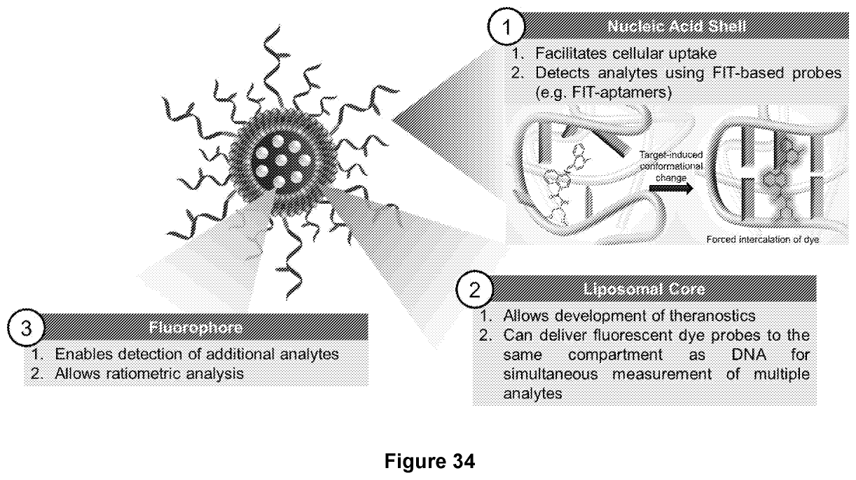

FIG. 35 depicts an exemplary design of a FIT-Flare highlighting its components and their significance to the detection scheme.

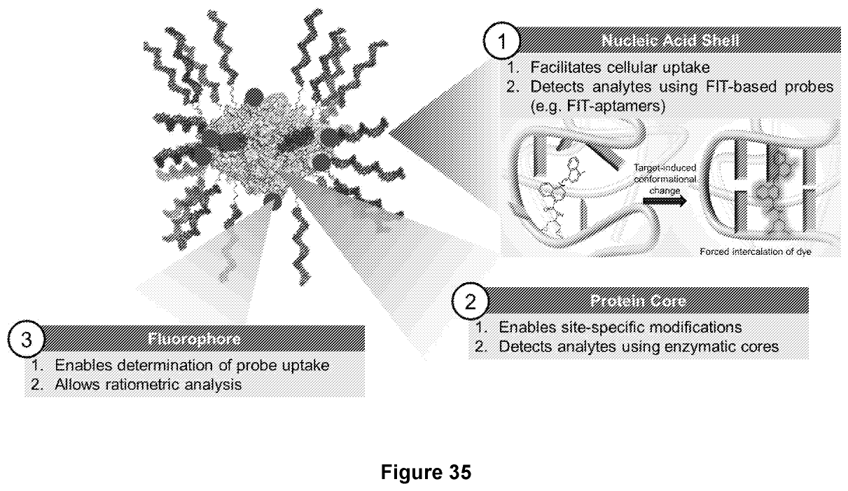

FIG. 36 depicts a general scheme for the synthesis of ProSNAs. (A) Surface cysteine groups are first modified with Alexa Fluor™ 647 C₂ maleimide (highlighted in red). Then, NHS-PEG4-azides are conjugated to the surface-accessible lysine residues (highlighted in blue). Finally, DBCO-terminated DNA is attached to the azide-modified proteins through copper-free click chemistry. Structures of (B) Alexa Fluor™ 647 C₂ maleimide, (C) NHS-PEG4-azide, and (D) DBCO-terminated DNA. It is noted that the scheme shown in FIG. 36 was used for the synthesis of β-gal ProSNAs. For the synthesis of GOx-SNAs, the procedure was slightly modified. In this case, Alexa Fluor™ 647 NHS ester was conjugated through lysine residues due to the lack of surface cysteines.

FIG. 37 shows a scheme for synthesis of a FIT flare as described herein.

FIG. 38 shows data from experiments using ratiometric FIT-Flares for absolute quantification of pH.

FIG. 39 shows data from experiments using FIT-Flares to report pH changes in cells.

FIG. 40 shows nonlimiting examples of how using a functional protein core expands the range of analytes that can be detected using methods of the disclosure.

FIG. 41 shows data from experiments showing that GOx-SNAs were able to detect glucose in vitro.

FIG. 42 shows data from experiments showing that GOx-SNAs selectively turn on for glucose.

FIG. 43 shows data from flow cytometry experiments showing that GOx-SNAs detected glucose in multiple cell lines.

FIG. 44 shows data demonstrating that blocking glucose receptors decreases glucose uptake which was sensed by the GOx-SNA. A difference in fluorescence was no longer seen between dye-treated and dye+SNA-treated MDA MB 231 cells, which indicated that the receptor blocking retarded glucose uptake and that the GOx-SNA sensed this change.

FIG. 45 shows data from experiments demonstrating that liposomal FIT-Flares change the uptake pathway of an ATP-sensitive dye that localizes to the mitochondria.

FIG. 46 shows data from experiments demonstrating that FIT-Flares are localized in the endo-lysosomes.

FIG. 47 shows data from experiments showing that FIT-Flares allowed organelle-specific analyte mapping.

### DETAILED DESCRIPTION

The present disclosure is directed to spherical nucleic acids (SNAs) comprising a nanoparticle core and an oligonucleotide attached thereto, use of the SNAs to, e.g., detect target analytes, and methods of making the SNAs. In any of the embodiments or aspects of the disclosure, a plurality of oligonucleotides is attached to the nanoparticle core.

As used in this specification and the appended claims, the singular forms “a,” “an,” and “the” include plural reference unless the context clearly dictates otherwise.

The terms “polynucleotide” and “oligonucleotide” are interchangeable as used herein.

As used herein, “duplex” refers to a region in two complementary or sufficiently complementary oligonucleotides that form base pairs with one another, either by Watson-Crick base pairing or any other manner that allows for a stabilized duplex between oligonucleotide strands that are complementary or sufficiently complementary. “Sufficiently complementary” refers to the degree of complementarity between two nucleotide sequences such that a stable duplex is formed under the conditions in which the duplex is used. In various embodiments, sufficiently complementary nucleotide sequences are sequences that are or are at least 50%, 55%, 60%, 65%, 70%, 75%, 80%, 85%, 90%, 95%, or 99% complementary within a duplex. In some embodiments, sufficiently complementary nucleotide sequences are sequences that are 100% complementary within a duplex. In further embodiments, two nucleotide sequences are sufficiently complementary when there are 1, 2, 3, 4, 5, 6, 7, 8, 9, 10 or more mismatches between the two nucleotide sequences.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

### Spherical Nucleic Acids

Spherical nucleic acids (SNAs) comprise a nanoparticle core and an oligonucleotide attached thereto. In any of the aspects or embodiments of the disclosure, an SNA comprises a nanoparticle core and a plurality of oligonucleotides attached thereto. Thus, in some embodiments, an SNA comprises a densely functionalized and highly oriented shell of oligonucleotides on the exterior surface of a nanoparticle core. In various embodiments, all of the oligonucleotides attached to a nanoparticle core are the same, or in the alternative, at least two oligonucleotides are different. In some embodiments, each oligonucleotide in the plurality of oligonucleotides attached to a nanoparticle core comprises a detectable marker. In any of the aspects or embodiments of the disclosure, each oligonucleotide in the plurality of oligonucleotides attached to a nanoparticle core comprises a detectable marker situated at an internal location within the oligonucleotide, wherein the detectable marker is a marker with internal rotation-dependent fluorescence. In some embodiments, one or more oligonucleotides in the plurality of oligonucleotides does not comprise a detectable marker. In some embodiments, each oligonucleotide in the plurality of oligonucleotides is an aptamer. In some embodiments, each oligonucleotide in the plurality of oligonucleotides is a FIT aptamer. In some embodiments, one or more oligonucleotides in the plurality of oligonucleotides attached to a nanoparticle core is an inhibitory oligonucleotide. The inhibitory oligonucleotide, in various embodiments, is antisense DNA, small interfering RNA (siRNA), an aptamer, a short hairpin RNA (shRNA), a DNAzyme, an aptazyme, or a combination thereof. In some embodiments, one or more oligonucleotides in the plurality of oligonucleotides attached to a nanoparticle core is an immunostimulatory oligonucleotide. The immunostimulatory oligonucleotide, in various embodiments, is a toll-like receptor (TLR) agonist. In further embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, one or more oligonucleotides in the plurality of oligonucleotides attached to a nanoparticle core is a toll-like receptor (TLR) antagonist. In some embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof.

Accordingly, in various aspects the present disclosure provides spherical nucleic acids comprising a nanoparticle core and an oligonucleotide attached thereto. In some aspects, the disclosure provides intracellular probes based on protein spherical nucleic acids (ProSNAs).^{14,15} This design allows analyte detection via a quencher-free approach using the nucleic acid and/or protein component. Additionally, this platform allows for the detection of intracellular analytes through binding-based or activity-based sensing (or both). ProSNAs are based on the SNA architecture and, in some embodiments, comprise a protein core functionalized with a dense shell of radially oriented oligonucleotides. The SNA architecture is ideally suited for making intracellular measurements as it is non-toxic to cells, elicits minimal immune response, can be taken up by cells without the need for transfection reagents, and is more resistant to nuclease degradation compared to traditionally used linear oligonucleotide probes.¹⁶ Additionally, it enables the intracellular delivery of functional proteins and confers stability against protease degradation.^{14,15}

SNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further aspects, the disclosure provides a plurality of SNAs, each SNA comprising one or more oligonucleotides attached thereto. Thus, in some embodiments, the size of the plurality of SNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of SNAs) of the SNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the SNAs, for example, the amount of surface area to which oligonucleotides may be attached as described herein. It will be understood that the foregoing diameters of SNAs can apply to the diameter of the nanoparticle core itself or to the diameter of the nanoparticle core and the one or more oligonucleotides attached thereto.

### Nanoparticle Core

As described herein, a SNA comprises a nanoparticle core and an oligonucleotide attached thereto. In general, nanoparticles contemplated by the disclosure include any compound or substance with a high loading capacity for an oligonucleotide as described herein, including for example and without limitation, a protein, a metal, a semiconductor, a liposomal particle, a polymer-based particle (e.g., a poly (lactic-co-glycolic acid) (PLGA) particle), insulator particle compositions, and a dendrimer (organic versus inorganic). Thus, in various embodiments, the nanoparticle core is organic (e.g., a liposome), inorganic (e.g., gold, silver, or platinum), porous (e.g., silica-based or metal organic-framework-based), or hollow. In any of the aspects or embodiments of the disclosure, the nanoparticle core is a protein core.

Thus, the disclosure contemplates nanoparticles that comprise a variety of inorganic materials including, but not limited to, metals, semi-conductor materials or ceramics as described in U.S. Patent Publication No 20030147966. For example, metal-based nanoparticles include those described herein. In various embodiments, the nanoparticle core is a metallic core, a semiconductor core, an insulator core, an upconverting core, a micellar core, a dendrimer core, a liposomal core, a polymer core, a metal-organic framework core, a protein core, or a combination thereof. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles. In some embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), chitosan, or a related structure. In some embodiments, the polymer is poly(lactic-co-glycolic acid) (PLGA).

Liposomal particles, for example as disclosed in International Patent Application No. PCT/US2014/068429 (incorporated by reference herein in its entirety) are also contemplated by the disclosure. Hollow particles, for example as described in U.S. Patent Publication Number 2012/0282186 (incorporated by reference herein in its entirety) are also contemplated herein. Liposomes of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. In various embodiments, the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, or a combination thereof.

In some embodiments, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, Agl, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, Agl, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992). In some embodiments, the nanoparticle is an iron oxide nanoparticle. In further embodiments, the nanoparticle core is gold, silver, platinum, aluminum, palladium, copper, cobalt, indium, cadmium selenide, iron oxide, fullerene, metal-organic framework, zinc sulfide, or nickel.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Suitable nanoparticles are also commercially available from, for example, Ted Pella, Inc. (gold), Amersham Corporation (gold) and Nanoprobes, Inc. (gold).

In some aspects of the disclosure, the nanoparticle core is a protein. As used herein, protein is used interchangeably with “polypeptide” and refers to one or more polymers of amino acid residues. In various embodiments of the disclosure, a protein core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically. Protein/oligonucleotide core-shell nanoparticles are also generally described in U.S. Patent Application Publication No. 2017/0232109, which is incorporated by reference herein in its entirety.

Proteins are understood in the art and include without limitation an enzyme, a therapeutic protein, a structural protein (e.g., actin), a defensive protein (e.g., an antibody), a storage protein (e.g., ovalbumin), a transport protein (e.g., hemoglobin), a hormone (e.g., insulin), a receptor protein (e.g., G-Protein Coupled Receptors), a motor protein (e.g., kinesin, dynein, or myosin), or a fluorescent protein. In some embodiments, the enzyme is glucose oxidase (GOx), cholesterol oxidase, luciferase, or creatinine deaminase. In further embodiments, the therapeutic protein is insulin, glucocerebrosidase, thrombin, Chorionic Gonadotropin, Antihemophilic factor, or a combination thereof. In various embodiments, proteins contemplated by the disclosure include without limitation those having catalytic, signaling, therapeutic, or transport activity.

Proteins of the present disclosure may be either naturally occurring or non-naturally occurring. Proteins optionally include a spacer as described herein.

Naturally occurring proteins include without limitation biologically active proteins (including antibodies) that exist in nature or can be produced in a form that is found in nature by, for example, chemical synthesis or recombinant expression techniques. Naturally occurring proteins also include lipoproteins and post-translationally modified proteins, such as, for example and without limitation, glycosylated proteins. Antibodies contemplated for use in the methods and compositions of the present disclosure include without limitation antibodies that recognize and associate with a target molecule either in vivo or in vitro.

Structural proteins contemplated by the disclosure include without limitation actin, tubulin, collagen, and elastin.

Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “peptide” typically refers to short (e.g., about 2-50 amino acids in length) polypeptides/proteins. Non-naturally occurring proteins are prepared, for example, using an automated protein synthesizer or, alternatively, using recombinant expression techniques using a modified polynucleotide which encodes the desired protein.

As used herein a “fragment” of a protein is meant to refer to any portion of a protein smaller than the full-length protein or protein expression product. As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it. As used herein a “variant” refers to a protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art. In various aspects, proteins are modified by glycosylation, pegylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic. By way of example, an endothelial growth factor mimetic is a peptide or protein that has a biological activity comparable to the native endothelial growth factor. The term further includes peptides or proteins that indirectly mimic the activity of a protein of interest, such as by potentiating the effects of the natural ligand of the protein of interest.

Proteins include antibodies along with fragments and derivatives thereof, including but not limited to Fab′ fragments, F(ab)2 fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

### Oligonucleotides

The disclosure provides spherical nucleic acids (SNAs) comprising a nanoparticle core and an oligonucleotide attached thereto. In any of the aspects of embodiments of the disclosure, the SNA comprises a nanoparticle core and a plurality of oligonucleotides attached thereto. The disclosure contemplates, in any aspects or embodiments described herein, the use of DNA oligonucleotides, RNA oligonucleotides, or a combination thereof. In any aspects or embodiments described herein, an oligonucleotide is single-stranded, double-stranded, or partially double-stranded. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}, >C═O, >C═NR^{H}, >C═S, —Si(R″)₂-, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}— CH₂—, —CH₂—NR^{H}—O—, —CH₂—NR^{H}—CO—, —O—NR^{H}— CH₂—, —O—NR^{H}, —O— CH₂—S—, —S— CH₂—O—, —CH₂— CH₂—S—, —O—CH₂— CH₂—S—, —S— CH₂—CH═ (including R⁵ when used as a linkage to a succeeding monomer), —S— CH₂— CH₂—, —S— CH₂— CH₂—O—, —S—CH₂—CH₂—S—, —CH₂—S— CH₂—, —CH₂—SO— CH₂—, —CH₂—SO₂— CH₂—, —O—SO—O—, —O—S(O)₂—O—, —O—S(O)₂— CH₂—, —O—S(O)₂—NR^{H}—, —NR^{H}—S(O)₂— CH₂—; —O—S(O)₂— CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O—CH₂CH₃)—O—, —O—PO(OCH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂— CH₂—, and —O—Si(R″)₂—O—; among which —CH₂—CO—NR^{H}—, —CH₂—NR^{H}—O—, —S— CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)20N(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 10 nucleotides to about 100 nucleotides in length. More specifically, an oligonucleotide of the disclosure is about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. In further embodiments, an oligonucleotide of the disclosure is about 5 nucleotides to about 1000 nucleotides in length. In further embodiments, an oligonucleotide of the disclosure is about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8,9, 10, 11, 12, 13, 14,15, 16, 17, 18, 19,20,21,22,23,24,25,26,27,28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length.

In some embodiments, the oligonucleotide is an aptamer. Accordingly, all features and aspects of oligonucleotides described herein (e.g., length, type (DNA, RNA, modified forms thereof, optional presence of spacer)) also apply to aptamers. Aptamers are oligonucleotide sequences that can be evolved to bind to various target analytes of interest. In some aspects, an aptamer of the disclosure is a FIT aptamer. As used herein, a “FIT aptamer” is an aptamer that comprises a detectable marker situated at an internal location within the aptamer. Thus, in some aspects the present disclosure provides a general design strategy that transduces an aptamer-target binding event into a fluorescence readout via the use of a viscosity-sensitive dye. Target binding to the aptamer leads to forced intercalation (FIT) of the dye between oligonucleotide base pairs, increasing its fluorescence by up to 20-fold. Using the forced intercalation approach, a “duplex-sensitive” dye is chemically attached as a base surrogate in an oligonucleotide sequence. The oligonucleotide sequence is a “recognition” sequence that can bind to a target analyte of interest, and binding of the recognition strand to the target restricts the rotation of the duplex-sensitive dye due to forced intercalation between the base pairs, and leads to a fluorescence turn on. As disclosed herein, the target analytes include but are not limited to a protein, an ion, a small molecule, a lipid, a carbohydrate, an oligosaccharide, a cell, or a combination thereof. In some embodiments, the target analyte is not a nucleic acid.

FIT-aptamers can report target presence through intramolecular conformational changes, sandwich assays, and target-templated reassociation of split-aptamers, showing that the most common aptamer-target binding modes can be coupled to a FIT-based readout. In some embodiments, this strategy is used to detect the formation of a metallo-base pair within a duplexed strand and is therefore attractive for screening for metal-mediated base pairing events. FIT-aptamers reduce false-positive signals typically associated with fluorophore-quencher based systems, quantitatively outperform FRET-based probes by providing up to 15-fold higher signal to background ratios, and allow rapid and highly sensitive target detection (nanomolar range) in complex media such as human serum. Taken together, FIT-aptamers are a new class of signaling aptamers which contain a single modification, yet can be used to detect a broad range of targets. In various embodiments, the disclosure contemplates the use of aptamers, such as FIT aptamers. In some aspects, the disclosure provides a composition comprising a spherical nucleic acid (SNA), wherein the SNA comprises a nanoparticle core and an aptamer attached thereto. In some aspects, the aptamer is a FIT aptamer as described herein. In various embodiments, an aptamer is a DNA oligonucleotide or a modified form thereof, an RNA oligonucleotide or a modified form thereof, or a combination thereof. Aptamers may be single stranded, double stranded, or partially double stranded. As described herein, a FIT aptamer is an aptamer that comprises a detectable marker situated at an internal location within the aptamer. In various embodiments, the detectable marker is situated at a position that is 1, 2, 3, 4, 5, 6, 7, 8, 9, 10, or more nucleotides from a terminus (i.e., 5′ or 3′ terminus) of the aptamer. In some embodiments, the detectable marker is situated at a position that is x nucleotides from a terminus (i.e., 5′ or 3′ terminus) of the aptamer, wherein x is an integer that is 1, n/2, or any integer between 1 and n/2, wherein n is (i) the length of the aptamer and (ii) an even number. In further embodiments, the detectable marker is situated at a position that is x nucleotides from a terminus (i.e., 5′ or 3′ terminus) of the aptamer, wherein x is an integer that is 1, (n+1)/2, or any integer between 1 and (n+1)/2, wherein n is (i) the length of the aptamer and (ii) an odd number. In some embodiments, the detectable marker is situated at about a midpoint along the length of the aptamer, wherein the nucleotide sequences on either side of the detectable marker are sufficiently complementary to form a duplex. In any of the aspects or embodiments of the disclosure, the FIT aptamer consists of one detectable marker. In some embodiments, the FIT aptamer comprises 1, 2, 3, 4, 5, 6, 7, 8, 9, 10, or more detectable markers. Methods of attaching detectable markers and additional moieties as described herein to an oligonucleotide are known in the art. It will be understood that, when more than one aptamer is used together (e.g., in a sandwich assay as described herein) each aptamer may be a different length, or some or all of the aptamers may be the same length.

Spacers. In some aspects, an oligonucleotide is attached to a nanoparticle through a spacer. “Spacer” as used herein means a moiety that serves to increase distance between the nanoparticle and the oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotide to perform an intended function (e.g., bind to a target analyte). In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

Oligonucleotide attachment to a nanoparticle. Oligonucleotides contemplated for use in the methods include those attached to a nanoparticle core through any means (e.g., covalent or non-covalent attachment). Regardless of the means by which the oligonucleotide is attached to the nanoparticle, attachment in various aspects is effected through a 5′ linkage, a 3′ linkage, some type of internal linkage, or any combination of these attachments. In some embodiments, the oligonucleotide is covalently attached to a nanoparticle. In further embodiments, the oligonucleotide is non-covalently attached to a nanoparticle.

Methods of attachment are known to those of ordinary skill in the art and are described in U.S. Publication No. 2009/0209629, which is incorporated by reference herein in its entirety. Methods of attaching RNA to a nanoparticle are generally described in PCT/US2009/65822, which is incorporated by reference herein in its entirety. Methods of associating oligonucleotides with a liposomal particle are described in PCT/US2014/068429, which is incorporated by reference herein in its entirety. Methods of attaching oligonucleotides to a protein core are described, e.g., in U.S. Patent Application Publication No. 2017/0232109 and Brodin et al., J Am Chem Soc. 137(47): 14838-41 (2015), which are incorporated by reference herein in their entirety.

Nanoparticle surface density. A surface density adequate to make the nanoparticles stable and the conditions necessary to obtain it for a desired combination of nanoparticles and oligonucleotides can be determined empirically. Generally, a surface density of at least about 2 pmoles/cm² will be adequate to provide stable nanoparticle-oligonucleotide compositions. In some aspects, the surface density is at least 15 pmoles/cm². Methods are also provided wherein the oligonucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm2, at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of oligonucleotide attached to the SNA is measured by the number of oligonucleotides attached to the SNA. With respect to the surface density of oligonucleotides attached to an SNA of the disclosure, it is contemplated that a SNA as described herein comprises about 1 to about 2,500, or about 1 to about 500 oligonucleotides on its surface. In various embodiments, a SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5,10, 20, 30, 40, 45, 50, 55, 60, 65, 70, 75, 80, 85, 90, 95, 100,105,110,115,120, 125, 130, 135, 140, 145,150, 155, 160, 165, 170,175, 180, 185, 190, 195, or 200 oligonucleotides in the shell of oligonucleotides attached to the nanoparticle core. In further embodiments, a SNA consists of 5, 10, 20, 30, 40, 45, 50, 55, 60, 65, 70, 75, 80, 85, 90, 95, 100, 105, 110, 115, 120,125, 130, 135, 140, 145,150, 155, 160, 165, 170,175, 180, 185, 190, 195, or 200 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20 or more oligonucleotides. In some embodiments, the shell of oligonucleotides attached to the nanoparticle core of the SNA consists of 2, 3, 4, 5, 6, 7, 8, 9, 10, 11, 12, 13, 14, 15, 16, 17, 18, 19, or 20 oligonucleotides.

### Detectable Markers

In any of the aspects or embodiments of the disclosure, an oligonucleotide comprises a detectable marker. In some embodiments, the oligonucleotide is an aptamer (e.g., a FIT aptamer). In various embodiments, an oligonucleotide (e.g., a FIT aptamer) comprises 2, 3, 4, 5, 6, 7, 8, 9, 10, or more detectable markers, which may be either all the same or one or more detectable markers may be different. In various embodiments, the one or more detectable markers is situated at any internal position with an oligonucleotide. In any of the aspects or embodiments of the disclosure, the detectable marker is a marker that exhibits internal rotation-dependent fluorescence or is viscosity-sensitive. In various embodiments, the detectable marker is thiazole orange (TO), quinoline blue, quinoline violet, thiazole red, a derivative thereof, or a cyanine derivative. In various embodiments, however, an oligonucleotide comprises a fluorophore that does not exhibit internal rotation-dependent fluorescence (e.g., fluorescein).

Methods of attaching a detectable marker to an oligonucleotide or to a nanoparticle core are known in the art and exemplified herein. In some embodiments, the detectable marker is situated at a position that is x nucleotides from a terminus (i.e., 5′ or 3′ terminus) of an aptamer, wherein x is an integer that is 1, n/2, or an integer between 1 and n/2, and n is (i) the length of the aptamer and (ii) an even number. In further embodiments, one or more detectable markers are situated at different positions within an aptamer, with each detectable marker being situated at a position that is x nucleotides from a terminus (i.e., 5′ or 3′ terminus) of the aptamer, wherein x is an integer that is 1, n/2, or an integer between 1 and n/2, and n is (i) the length of the aptamer and (ii) an even number. In some embodiments, the detectable marker is situated at a position that is x nucleotides from a terminus (i.e., 5′ or 3′ terminus) of an aptamer, wherein x is an integer that is 1, (n+1)/2 or any integer between 1 and (n+1)/2, and n is (i) the length of the aptamer and (ii) an odd number. In further embodiments, one or more detectable markers are situated at different positions within an aptamer, with each detectable marker being situated at a position that is x nucleotides from a terminus of the aptamer, wherein x is an integer that is 1, (n+1)/2 or any integer between 1 and (n+1)/2, and n is (i) the length of the aptamer and (ii) an odd number.

In some embodiments, a detectable marker is associated with the nanoparticle core. For example and without limitation, a nanoparticle core of the disclosure (e.g., a protein core, a liposome, a polymer core) may be labeled with a fluorophore. In further embodiments, and as described above, an oligonucleotide that is attached to the nanoparticle comprises a fluorophore that does not exhibit internal rotation-dependent fluorescence. In some embodiments, both the nanoparticle core and an oligonucleotide attached thereto comprise a fluorophore and the fluorophore attached to the nanoparticle core may be the same or different than the fluorophore attached to the oligonucleotide attached to the nanoparticle core. In various embodiments, the fluorophore is fluorescein derivatives, rhodamine derivatives, cyanine dyes, AlexaFluor dyes, ATTO dyes.

### Agents

In various aspects, the disclosure contemplates that methods of detecting a target analyte include or further comprise contacting a target analyte with an agent. As described herein, in some embodiments the nanoparticle core is a protein core. In some embodiments, contacting the protein core with the target analyte results in a detectable change. In some embodiments, the protein core comprises an enzyme that interacts with and allows detection of the target analyte. The enzyme, in various embodiments, is glucose oxidase (GOx), cholesterol oxidase, luciferase, or creatinine deaminase. In some embodiments, after the target analyte has been contacted with the protein core, contact between the target analyte and an agent occurs and the target analyte is detectable after contacting the target analyte with the agent. In various embodiments, the agent is associated with the external side of the nanoparticle core, encapsulated in the nanoparticle core, associated with an oligonucleotide that is attached to the protein core, added exogenously, or a combination thereof.

In various embodiments, the agent is a small molecule. In some embodiments, the small molecule is a dye or a luminophore. In further embodiments, the dye is a normalizing dye, a dye that localizes to an organelle, or a dye that detects an additional analyte not being sensed by either the nanoparticle core or the one or more oligonucleotides attached thereto.

### Target Analytes

In various aspects and embodiments, the disclosure provides compositions and methods of detecting target analytes. Target analytes contemplated by the disclosure include without limitation a protein, an ion, a small molecule, a lipid, a carbohydrate, an oligosaccharide, a cell, an oligonucleotide, or a combination thereof.

In some embodiments, the ion is an anion or a cation. In some embodiments, ions contemplated by the disclosure are metal ions. In further embodiments, the metal ion is a mercury ion, a copper ion, a silver ion, zinc ion, gold ion, manganese ion, or a combination thereof. In some embodiments, the ion is a hydrogen ion. In further embodiments, the change in the detectable marker is indicative of a pH change.

In some embodiments, the target analyte is a protein as described herein. Thus, in various aspects and embodiments of the disclosure, a protein functions as a protein core of a SNA and another protein is the target analyte to be detected. In some embodiments, a protein functions as a protein core of a SNA and a different protein is the target analyte to be detected.

The term “small molecule,” as used herein, refers to a chemical compound, or any other low molecular weight organic compound, either natural or synthetic. By “low molecular weight” is meant compounds having a molecular weight of less than 1500 Daltons, typically between 100 and 700 Daltons.

In some embodiments, the target analyte is an oligonucleotide. In some embodiments, it is contemplated that target oligonucleotide binding to a FIT aptamer duplex forms a “triplex structure,” causing a conformational change in the detectable marker that results in detection of the marker.

Lipids are understood in the art. Non-limiting examples include tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, polyunsaturated sterols of different lengths, saturation states, saturated C8-C22 fatty acids, saturated C8-C22 ether derivatives of glycerol, saturated and unsaturated amide derivatives of C8-C22 fatty acids and mono- and 1,2- or 1,3-di-amino glycerols, derivatives thereof, or a combination thereof.

Carbohydrates are known in the art. Non-limiting examples include sucrose, xylose, mannose, fructose, maltose, lactose, galactose, derivatives thereof, or a combination thereof.

Oligosaccharides are understood in the art. Non-limiting examples include cellobiose, cellodextrin, B-cyclodextrin, indigestible dextrin, gentio-oligosaccharide, gluco-oligosaccharide, isomaltoligosaccharide, isomaltose, isomatriose, panose, leucrose), Palatinose, cyananderose, D-agatose, D-lyxo-hexulose, lactosucrose, α-galactooligosaccharide, β-galactooligosaccharide, Transgalactooligosaccharides, lactulose, 4′-galatosyllactose, synthetic galactooligosaccharides, fructans-Levan-type, frustans-Inutin-type, 1f-β-fructofuranosylnystose (1f-β-fructofuranosylnystose), xylooligosaccharide, raffinose (lafinose), lactosucrose and arabino-oligosaccharides, derivatives thereof, or a combination thereof.

### Therapeutic Agents

In some embodiments, a SNA of the disclosure further comprises a therapeutic agent. Thus, the disclosure provides SNAs having theranostic capabilities. The therapeutic agent acts before or after the contacting of the SNA with one or more target analytes. In some embodiments, the therapeutic agent is associated with the nanoparticle core. In some embodiments, the therapeutic agent is encapsulated in the nanoparticle core or is attached to the external side of the nanoparticle core. In further embodiments, the therapeutic agent is associated with the oligonucleotide. In various embodiments, the therapeutic agent is a chemotherapeutic agent or an anti-diabetes drug.

By way of nonlimiting example, a SNA of the disclosure may be designed to detect a target analyte in a cancer cell, or the target analyte may be a cancer cell, such that detection of the target analyte results in an interaction of a candidate chemotherapeutic agent with the target analyte and the effects of the candidate chemotherapeutic agent on the cancer cell are measured. Use of such a method allows for high throughput screens testing the effectiveness of candidate therapeutic agents, and such screens are contemplated herein.

### Methods of Target Analyte Detection

The present disclosure is directed to compositions and methods for detecting various target analytes. The methods generally comprise contacting the target analyte with a spherical nucleic acid (SNA), wherein the SNA comprises a nanoparticle core and an oligonucleotide and the contacting results in a detectable change. In various embodiments, the target analyte is in a cell and the contacting occurs intracellularly. In some embodiments, the target analyte is outside a cell and the contacting occurs extracellularly. The SNAs of the disclosure are highly tailorable, such that they can be designed to detect a single target analyte or multiple different target analytes depending on the selection of the nanoparticle core, the one or more oligonucleotides attached thereto, and any agents that are optionally associated with the SNA or added exogenously. Thus, in various aspects and embodiments of the disclosure, and as described herein, a target analyte may be detected by virtue of its interaction with the nanoparticle core, one or more oligonucleotides attached to the nanoparticle core, an agent, or a combination thereof. In addition, the detectable change that occurs upon interaction of the SNA and/or agent with the target analyte is proportional to the concentration of the target analyte. In any of the aspects or embodiments of the disclosure, the detectable change is an increase in fluorescence or luminescence.

Accordingly, in some aspects the disclosure provides a method for detecting a target analyte comprising the step of contacting the target analyte with a spherical nucleic acid (SNA), the SNA comprising a nanoparticle core and an oligonucleotide attached thereto, wherein the oligonucleotide comprises a detectable marker situated at an internal location within the oligonucleotide, wherein the contacting results in binding of the target analyte to the oligonucleotide, and wherein the binding results in restriction of internal rotation of the marker, resulting in a detectable change and thereby detecting the target analyte. In some embodiments, a plurality of the oligonucleotides is attached thereto. In some embodiments, the detectable change is an increase in fluorescence. As described herein, in some embodiments the oligonucleotide is an aptamer (e.g., a FIT aptamer). In some embodiments, the oligonucleotide is a FIT oligonucleotide. In some embodiments, target analyte binding to the oligonucleotide results in forced intercalation (FIT) of the marker between base pairs of the oligonucleotide. In some embodiments, the detectable marker is a marker with internal rotation-dependent fluorescence. In some embodiments, the detectable marker is a viscosity-sensitive marker. In further embodiments, the detectable marker is thiazole orange (TO), quinoline blue, quinoline violet, thiazole red, a derivative thereof, or a cyanine derivative. In any of the aspects or embodiments of the disclosure, the detectable change is proportional to concentration of the target analyte. In various aspects, the disclosure provides FIT oligonucleotides (e.g., FIT aptamers) that are useful for detecting presence of a target analyte through, for example and without limitation, intramolecular conformational changes, sandwich assays, and target-templated reassociation of split-aptamers, each as described herein.

In some aspects, the disclosure also provides methods for detecting a target analyte comprising the step of contacting the target analyte with a spherical nucleic acid (SNA) and an agent as described herein, the SNA comprising a protein core and an oligonucleotide attached thereto, wherein the contacting of the protein core with the target analyte results in a change in the target analyte that is detectable by the agent, thereby detecting the target analyte. In some embodiments, the SNA comprises a plurality of oligonucleotides attached thereto. In some embodiments, the oligonucleotide does not comprise a detectable marker with internal rotation-dependent fluorescence. In some embodiments, none of the plurality of oligonucleotides attached to a nanoparticle core (e.g., a protein core) comprise a detectable marker with internal rotation-dependent fluorescence. In some embodiments, the change that is detectable by the agent is a fluorescence change or a luminescence change. In some embodiments, the protein core comprises an enzyme. In some embodiments, the enzyme is glucose oxidase (GOx), cholesterol oxidase, luciferase, or creatinine deaminase. In various embodiments, the agent is associated with the external side of the nanoparticle core, encapsulated in the nanoparticle core, associated with the oligonucleotide or with one or more of the plurality of oligonucleotides, is added exogenously, or a combination thereof. In some embodiments, the agent is a small molecule. In some embodiments, the small molecule is a dye or a luminophore. In some embodiments, the dye is a normalizing dye, a dye that localizes to an organelle, or a dye that detects an additional analyte not being sensed by either the nanoparticle core or the one or more oligonucleotides attached thereto. In some embodiments, the oligonucleotide or one or more oligonucleotides in the plurality of oligonucleotides is an inhibitory oligonucleotide. In some embodiments, the inhibitory oligonucleotide is antisense DNA, small interfering RNA (siRNA), an aptamer, a short hairpin RNA (shRNA), a DNAzyme, or an aptazyme. In some embodiments, the oligonucleotide or one or more oligonucleotides in the plurality of oligonucleotides is an immunostimulatory oligonucleotide. In some embodiments, the immunostimulatory oligonucleotide is a toll-like receptor (TLR) agonist. In further embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the oligonucleotide or one or more oligonucleotides in the plurality of oligonucleotides is a toll-like receptor (TLR) antagonist. In some embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some embodiments, the target analyte is a protein, an ion, a small molecule, a lipid, a carbohydrate, an oligosaccharide, a cell, or a combination thereof. In some embodiments, the target analyte is not a nucleic acid. In some embodiments, the ion is a metal ion. In some embodiments, metal ion is a mercury ion, a copper ion, a silver ion, zinc ion, gold ion, manganese ion, or a combination thereof. In some embodiments, the ion is a hydrogen ion. In some embodiments, the detectable change is indicative of a pH change.

Intramolecular conformational changes. As described herein, FIT oligonucleotides (e.g., FIT aptamers) comprise a detectable marker situated at an internal location within the oligonucleotide. In some aspects, the disclosure provides a method of detecting a target analyte comprising the step of contacting the target analyte with a spherical nucleic acid (SNA), wherein the SNA comprises a nanoparticle core and an oligonucleotide attached thereto, wherein the oligonucleotide comprises a detectable marker situated at an internal location within the oligonucleotide, wherein the contacting results in binding of the target analyte to the oligonucleotide, wherein target analyte binding to the oligonucleotide results in restriction of internal rotation of the marker, resulting in a detectable change in the marker. In some embodiments, target analyte binding to the oligonucleotide results in forced intercalation (FIT) of the marker between oligonucleotide base pairs of the oligonucleotide. In some embodiments, the oligonucleotide is a FIT aptamer and target analyte binding to the FIT aptamer results in intramolecular duplex formation in the aptamer. In further embodiments, target analyte binding to the aptamer results in triplex or tetraplex formation in the aptamer.

Sandwich Assays/Split Aptamers. A sandwich assay generally refers to the use of more than one aptamer to bind to a target analyte, wherein each of the more than one aptamers binds to a different binding site on the target analyte. Thus, in some embodiments, a target analyte having two or more binding sites is contacted with two different aptamers that bind independently to different binding sites on the target. The two different aptamers are attached to the same nanoparticle core, or the two different aptamers are attached to different nanoparticle cores. Accordingly, in some aspects, the disclosure provides a method for detecting a target analyte comprising the step of contacting the target analyte with a spherical nucleic acid (SNA), the SNA comprising a nanoparticle core and a plurality of oligonucleotides attached thereto, the plurality of oligonucleotides comprising (a) an aptamer or portion thereof comprising (i) nucleotide sequence X, (ii) nucleotide sequence Y which binds to the target analyte, either alone or in combination with nucleotide sequence Y′, and (iii) a detectable marker situated at an internal location within the aptamer, and (b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y, wherein the contacting results in hybridization of nucleotide sequence X with nucleotide sequence X′ and binding of the target analyte with nucleotide sequence Y and nucleotide sequence Y′, wherein the binding of nucleotide sequence X with nucleotide sequence X′ and the target analyte with nucleotide sequence Y and nucleotide sequence Y′ result in restriction of internal rotation of the marker, resulting in a detectable change and thereby detecting the target analyte. In some embodiments, nucleotide sequence Y and nucleotide sequence Y′ bind to different binding sites of the target analyte. In some embodiments, nucleotide sequence Y and nucleotide sequence Y′ together bind to the same binding site of the target analyte. In some embodiments, binding of nucleotide sequence X with nucleotide sequence X′ and the target analyte with nucleotide sequence Y and nucleotide sequence Y′ result in forced intercalation (FIT) of the marker between oligonucleotide base pairs of the aptamer and the additional aptamer. In some embodiments, the detectable marker is situated at a position that is x nucleotides from a terminus of the aptamer, wherein x is an integer that is 1, n/2, or any integer between 1 and n/2, wherein n is (i) the length of the aptamer and (ii) an even number. In some embodiments, the detectable marker is situated at a position that is x nucleotides from a terminus of the aptamer, wherein x is an integer that is 1, (n+1)/2, or any integer between 1 and (n+1)/2, wherein n is (i) the length of the aptamer and (ii) an odd number. In some embodiments, the detectable marker is situated at a position that is 1, 2, 3, 4, 5, 6, 7, 8, 9, 10, or more nucleotides from a terminus of the aptamer.

In some aspects, methods of the disclosure include the use of split aptamers. In some embodiments, it is contemplated that a target analyte has only a single binding site such that it may be bound by only a single aptamer. Split aptamer methods involve the use of a single aptamer sequence that is split to create two aptamer oligonucleotides, wherein each aptamer oligonucleotide comprises a portion that binds to the single binding site on the target analyte. The single aptamer may be split into two portions and optional additional nucleotides appended to each of the two portions, wherein the appended nucleotide sequences are sufficiently complementary to hybridize to each other. In various embodiments, the two portions, each with optional additional appended portions, are attached to the same nanoparticle core. Alternatively, the two portions, each with optional additional appended portions, are attached to separate nanoparticle cores. In further embodiments, one portion with optional additional appended portion is attached to a nanoparticle core and the other portion with optional additional appended portion is not attached to a nanoparticle core. Accordingly, in some aspects the disclosure provides a method of detecting a target analyte comprising the step of contacting the target analyte with a spherical nucleic acid (SNA), the SNA comprising a nanoparticle core and a plurality of oligonucleotides attached thereto, the plurality of oligonucleotides comprising (a) an aptamer or portion thereof comprising (i) nucleotide sequence X, (ii) nucleotide sequence Y which binds to the target analyte, either alone or in combination with nucleotide sequence Y′, and (iii) a detectable marker situated at an internal location within the aptamer, and (b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y, wherein the contacting results in hybridization of nucleotide sequence X with nucleotide sequence X′ and binding of the target analyte with nucleotide sequence Y and nucleotide sequence Y′, wherein the binding of nucleotide sequence X with nucleotide sequence X′ and the target analyte with nucleotide sequence Y and nucleotide sequence Y′ result in restriction of internal rotation of the marker, resulting in a detectable change and thereby detecting the target analyte. In some embodiments, nucleotide sequence Y and nucleotide sequence Y′ bind to different binding sites of the target analyte. In some embodiments, nucleotide sequence Y and nucleotide sequence Y′ together bind to the same binding site of the target analyte. In some embodiments, nucleotide sequence Y will not bind to the target analyte in the absence of nucleotide sequence Y′ also binding to the target analyte. In some embodiments, binding to the target analyte requires both portions of the aptamer.

### Identification of Novel Recognition Sequences

There are desirable target analytes for which no known recognition sequence exists. Alternatively, a recognition sequence for a target analyte may have been identified but it is not as efficient as would be desired. The present disclosure advantageously provides methods to identify novel recognition sequences for target analytes of interest. Thus, in some aspects the disclosure provides a method for identifying a nucleotide recognition sequence that is useful to detect a target analyte comprising the steps of contacting the target analyte with a spherical nucleic acid (SNA), the SNA comprising a nanoparticle core and an aptamer attached thereto, wherein the aptamer comprises a candidate nucleotide sequence and a detectable marker situated at an internal location within the aptamer, wherein binding of the candidate nucleotide sequence to the target analyte results in an increase in fluorescence due to restriction of internal rotation of the detectable marker; comparing fluorescence before and after the contacting, and identifying the candidate nucleotide sequence as the nucleotide recognition sequence from an increase in fluorescence after the contacting. In some embodiments, a plurality of the aptamers is attached thereto. In some embodiments, target analyte binding to the aptamer results in forced intercalation (FIT) of the marker between oligonucleotide base pairs of the aptamer. In some embodiments, the method is contemplated for use in high-throughput screens in which a plurality of populations of SNAs are constructed and tested. In this way, a multitude of candidate aptamers having different nucleotide sequences can be tested and recognition sequences having varying affinities for a target analyte can be identified.

### Use of SNAs in Gene Regulation/Therapy

It is contemplated that in any of the aspects or embodiments of the disclosure, a SNA as disclosed herein possesses the ability to regulate gene expression in addition to the ability to detect a target analyte. Thus, in some embodiments, a SNA of the disclosure comprises an oligonucleotide having gene regulatory activity (e.g., inhibition of target gene expression or target cell recognition). Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide. In various aspects, the methods include use of an oligonucleotide sufficiently complementary to a target polynucleotide as described herein.

Accordingly, methods of utilizing a SNA of the disclosure in gene regulation therapy are provided. This method comprises the step of hybridizing a polynucleotide encoding the gene with one or more oligonucleotides complementary to all or a portion of the polynucleotide, wherein hybridizing between the polynucleotide and the oligonucleotide occurs over a length of the polynucleotide with a degree of complementarity sufficient to inhibit expression of the gene product. The inhibition of gene expression may occur in vivo or in vitro.

The inhibitory oligonucleotide utilized in the methods of the disclosure is either RNA, DNA, or a modified form thereof. In various embodiments, the inhibitory oligonucleotide is antisense DNA, small interfering RNA (siRNA), a aptamer, a short hairpin RNA (shRNA), a DNAzyme, or an aptazyme.

### Use of SNAs in Immune Regulation

Toll-like receptors (TLRs) are a class of proteins, expressed in sentinel cells, that plays a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotide are located inside special intracellular compartments, called endosomes. The mechanism of modulation of TLR 4, TLR 8 and TLR 9 receptors is based on DNA-protein interactions.

Synthetic immunostimulatory oligonucleotides that contain CpG motifs that are similar to those found in bacterial DNA stimulate a similar response of the TLR receptors. Therefore immunomodulatory oligonucleotides have various potential therapeutic uses, including treatment of immune deficiency and cancer. Thus, in some embodiments, a SNA of the disclosure comprises an oligonucleotide that is a TLR agonist.

In further embodiments, down regulation of the immune system would involve knocking down the gene responsible for the expression of the Toll-like receptor. This antisense approach involves use of a SNA of the disclosure comprising a specific inhibitory oligonucleotide to knock down the expression of any toll-like protein. For example, down regulation of a gene responsible for the expression of a Toll-like receptor may be performed using a SNA as described herein. In further embodiments, a SNA of the disclosure comprises a TLR antagonist oligonucleotide.

Accordingly, in some embodiments, methods of utilizing SNAs as described herein for modulating toll-like receptors are disclosed. The method either up-regulates or down-regulates or antagonizes the Toll-like-receptor activity through the use of a TLR agonist or a TLR antagonist, respectively. The method comprises contacting a cell having a toll-like receptor with a SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Compositions

The disclosure also provides compositions that comprise a SNA of the disclosure, or a plurality thereof. In some embodiments, the composition further comprises a pharmaceutically acceptable carrier. The term “carrier” refers to a vehicle within which the SNA as described herein is administered to a subject. Any conventional media or agent that is compatible with the SNAs according to the disclosure can be used. The term carrier encompasses diluents, excipients, adjuvants and a combination thereof.

### EXAMPLES

The following examples demonstrate methods described herein for the chemical analysis of live cells based on protein spherical nucleic acids (ProSNAs). The ProSNA architecture enables analyte detection via the highly programmable nucleic acid shell and/or a functional protein core. As a proof-of-concept, an i-motif was used as the nucleic-acid recognition element to probe pH in living cells. By interfacing the i-motif with a forced-intercalation readout, a quencher-free approach that is resistant to false-positive signal was introduced. Using glucose oxidase as a functional protein core, activity-based, amplified sensing of glucose was demonstrated. This enzymatic system afforded greater than 100-fold fluorescence turn-on in buffer, was selective for glucose in the presence of close analogs (i.e., glucose-6-phosphate), and detected glucose above a threshold concentration of approximately 5 μM, which enables the study of relative changes in intracellular glucose concentrations.

### Example 1

### Materials and Methods

### Oligonucleotide Design, Synthesis, Purification, and Characterization

### Design

Three types of spherical nucleic acid (SNA)-based constructs were used in the study:

I. Gold NanoFlares (NFs)
a. With a pH-sensitive i-motif sequence as the recognition strand
b. With a pH-insensitive control sequence as the recognition strand

II. β-galactosidase (β-gal) SNAs
a. With a pH-sensitive i-motif sequence as the “FIT-aptamer”. This construct was referred to as ProTOn
b. With a pH-insensitive control sequence

III. Glucose oxidase SNAs (GOx-SNAs)

The DNA sequences used in designing the SNAs are provided in Table 1. D denotes the location of the forced intercalation dye thiazole orange (TO) in the sequence. Note that the 7th T from the 5′ end in the design of GOx-SNAs is modified with an amino group (amino-modifier C2 dT).

TABLE 1
Oligonucleotide sequences used in this study

| | SEQUENCE | SEQ |
|---|---|---|
| ABBREVIATION | (FROM 5′ END TO 3′ END) | ID NO. |
| Gold NF | CCC TAA CCC TAA CCC TAA CCC CY5 | 1 |
| i-motif | T₁₅-SH | |
| recognition | | |
| Gold NF | TTT CTA TCG CGT ACA ATC TGC CY5 | 2 |
| control | T₁₅-SH | |
| recognition | | |
| Gold NF | CY3-A₆ AGG GTT AGG GTT A | 3 |
| i-motif | | |
| flare | | |
| Gold NF | CY3-A₆ AGC AGA TTG TAC G | 4 |
| control | | |
| flare | | |
| ProSNA | DBCO TEG-T₁₅ C₄ TAA CDCC TAA C₄ | 5 |
| i-motif | TAA CTCC | |
| ProSNA | DBCO TEG-T₁₅ T₄ TTT TDTT TTT T₄ | 6 |
| control | TTT TTTT | |
| GOx DNA | DBCO TEG-T₁₃ | 7 |

Cy3 denotes cyanine-3.
Cy5 denotes cyanine-5.
DBCO-TEG denotes 10-(6-oxo-6-(dibenzo[b,f]azacyclooct-4-yn-1-yl)-capramido-N-ethyl)-O-triethyleneglycol-1-[(2-cyanoethyl)-(N,N-diisopropyl)]-phosphoramidite.

### Synthesis, Purification, and Characterization

All reagents for DNA synthesis were purchased from Glen Research. Oligonucleotides were synthesized using solid-phase phosphoramidite coupling chemistry. Universal or thiolated controlled pore glass (CPG) beads were used as the solid support. Synthesis was performed either using a MerMade12 (MM12, BioAutomation Inc., Plano, Tex., USA) or an ABI 394 instrument at 5 or 10 pmol scales. The oligonucleotides were then cleaved from the CPG beads using standard deprotection techniques (4 h at 55° C. or 16 h at room temperature using 2 mL of 30% ammonium hydroxide). An Organomation® Multivap® Nitrogen Evaporator was then used to evaporate off the ammonia. The remaining solution was adjusted to 2 mL in volume using nanopure water and filtered through a 0.2 μM syringe filter to remove the CPG beads. The filtrate was subjected to reverse phase high-performance liquid chromatography (RP-HPLC, Varian ProStar 210, Agilent Technologies Inc., Palo Alto, Calif., USA) to isolate the product. A C4 or C18 column and a gradient of 0 to 75% B over 45 min (A=triethylammonium acetate buffer, B=acetonitrile) were used. The collected fractions for sequences terminating in a 4,4′-dimethoxytrityl (DMTr) group were lyophilized and re-dissolved in 20% acetic acid for 1 h for detritylation. The cleaved DMTr group was removed by ethyl acetate extraction (3 times). The remaining acidic solution was lyophilized and re-dissolved in water. Sequences not terminating in a DMTr group did not require treatment with acetic acid. Matrix-assisted laser desorption ionization time-of-flight mass spectrometry (MALDI-TOF MS) was used to identify the product. The concentration (c) of the final product was determined using UV-vis spectroscopy. Specifically, c=A/E where A is the absorbance measured and E is the extinction coefficient of the oligonucleotide at 260 nm obtained from the IDT Oligo Analyzer Tool.

### Synthesis and Characterization of Thiazole Orange (TO)

### General Methods

All of the chemicals, reagents, and solvents were purchased as reagent grade from Sigma-Aldrich and used as received unless otherwise stated. Glassware and stir bars were oven-dried at 180° C. prior to use. Flash chromatography was performed with SiO₂ (230-400 mesh ASTM, 0.040-0.063 mm; Fluka). Deuterated solvents were purchased from Cambridge Isotope Laboratories and used as received. ¹H and ¹³C NMR spectra were recorded on a Bruker Avance 400 MHz NMR spectrometer at 298 K, and chemical shifts (δ) are given in parts per million. ¹H NMR spectra were referenced to residual proton resonances in the deuterated solvents (methanol-d4=δ 3.31), while absolute referencing was applied for heteronuclear NMR spectra (—C=25.145020). N-carboxylmethyl-4-methquinolinium bromide was synthesized following literature procedures [Bethge, L.; Jarikote, D. V.; Seitz, O. New Cyanine Dyes as Base Surrogates in PNA: Forced Intercalation Probes (FIT-Probes) for Homogeneous SNP Detection. Bioorg. Med. Chem. 2008, 16 (1), 114-125].

### Synthesis of Thiazole Orange Derivative

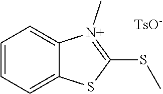

3-Methyl-2-(methylthio)-benzothiazolium tosylate was synthesized as described previously with slight modifications [Bethge, L.; Jarikote, D. V.; Seitz, O. New Cyanine Dyes as Base Surrogates in PNA: Forced Intercalation Probes (FIT-Probes) for Homogeneous SNP Detection. Bioorg. Med. Chem. 2008, 16 (1), 114-125]. Solid 2-methylthiobenzothiazole (4.12 g, 22.7 mmol, 1 equiv) was added to neat methyl p-toluenesulfonate (4.66 g, 25 mmol, 1.1 equiv) in an oven-dried round bottom flask and heated to reflux at 130° C. for 1 h. The reaction mixture was cooled to 70° C., and acetone was added till creamy precipitates formed. The reaction was then refluxed at 70° C. After 30 min, the mixture was cooled to room temperature, filtered, and washed with acetone. The creamy precipitates collected were dried in vacuo to yield the product (8.11 g, 22.1 mmol, isolated yield=97%). ¹H NMR (400 MHz, methanol-d₄) δ 8.20 (ddd, J=8.2, 1.3, 0.7 Hz, 1H), 8.05 (dt, J=8.6, 0.8 Hz, 1H), 7.82 (ddd, J=8.5, 7.3, 1.2 Hz, 1H), 7.71 (ddd, J=8.3, 7.3, 1.0 Hz, 1H), 7.69-7.61 (m, 2H), 7.22-7.14 (m, 2H), 4.12 (s, 3H), 3.10 (s, 3H), 2.33 (s, 3H). ¹³C NMR (101 MHz, MeOD) δ 181.12, 141.97, 141.63, 139.45, 128.62, 127.78, 127.65, 126.41, 124.80, 122.53, 114.37, 34.77, 19.20, 16.42.

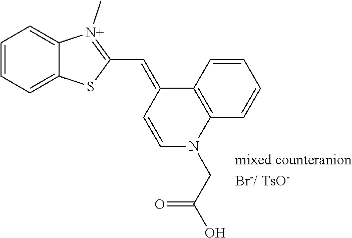 [SMILES: C[n+]1c(/C=C2\C=CN(CC(=O)O)c3ccccc32)sc2ccccc21]

Carboxymethylated thiazole orange was synthesized as described previously with slight modifications [Bethge, L.; Jarikote, D. V.; Seitz, O. New Cyanine Dyes as Base Surrogates in PNA: Forced Intercalation Probes (FIT-Probes) for Homogeneous SNP Detection. Bioorg. Med. Chem. 2008, 16 (1), 114-125]. N-carboxylmethyl-4-methquinolinium bromide (1.52 g, 5.39 mmol, 1.25 equiv) and 3-methyl-2-(methylthio)-benzothiazolium tosylate (1.58 g, 4.31 mmol, 1 equiv) were dissolved in dichloromethane. Triethylamine (1.5 mL, 10.8 mmol, 2.5 equiv) was then added. The reaction mixture, which turned dark red immediately, was stirred in the dark at room temperature for 16 h. The reaction mixture was dried on a rotary evaporator to give a red residue, which was dissolved in 325 mL of boiling methanol. 815 mL of water was then added to the red solution, which was stored at 4° C. for 3 days during crystallization. The red precipitate formed was collected by filtration, washed with a small amount of cold water, and dried in vacuo to give a red powder (1.64 g, 3.83 mmol, 89% isolated yield). Note that the product gradually decomposes in solution if it is exposed to light. ¹H NMR (400 MHz, methanol-d₄) δ 8.66-8.60 (m, 1H), 8.34 (d, J=7.2 Hz, 1H), 7.96-7.86 (m, 2H), 7.75-7.56 (m, 4H), 7.49 (dd, J=7.2, 2.3 Hz, 1H), 7.46-7.37 (m, 1H), 6.93 (s, 1H), 5.17 (s, 2H), 4.00 (s, 3H).

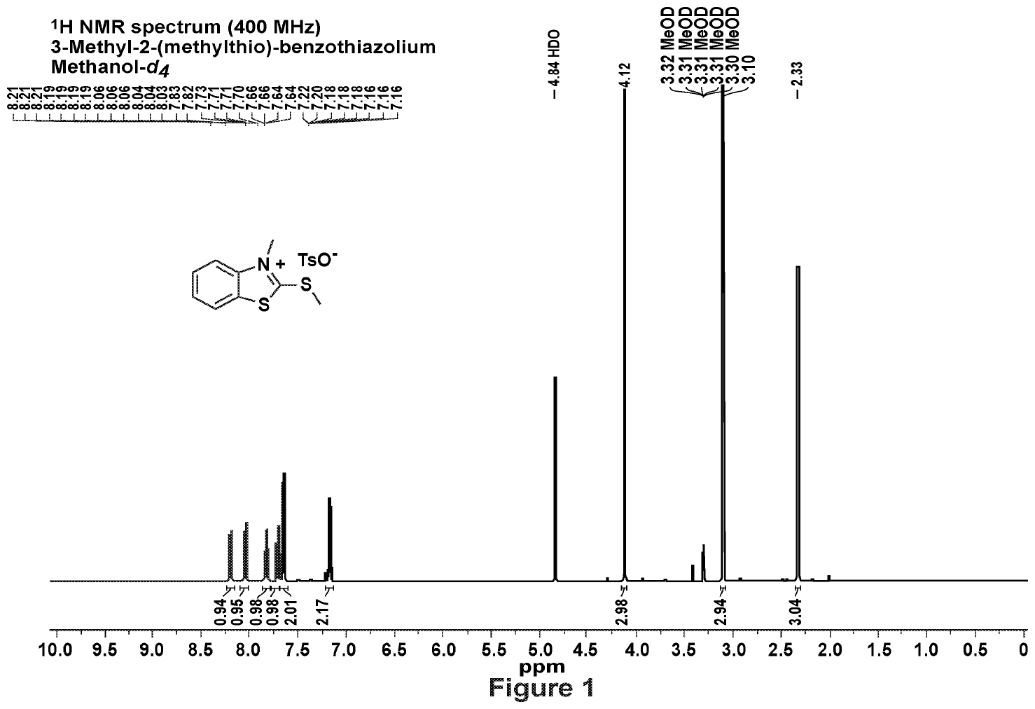

Resulting NMR spectra are shown in FIGS. 1-3.

### Coupling of to to Oligonucleotide Probes

Thiazole orange was conjugated to DNA sequences composed of an amino-modifier (N-trifluoroacetyl serinol phosphoramidite) according to a previously reported protocol [Ebrahimi, S. B.; Samanta, D.; Cheng, H. F.; Nathan, L. I.; Mirkin, C. A. Forced Intercalation (FIT)-Aptamers. J. Am. Chem. Soc. 2019, 141 (35), 13744-13748]. In a typical reaction, thiazole orange (5 pmol), pyridinium para-toluene sulfonate (5 pmol), N-hydroxysuccinimide (25 pmol), and 1-Ethyl-3-(3- dimethylaminopropyl) carbodiimide (50 pmol) were dissolved in 250 μL dimethylformamide and shaken for 10 min at 30° C. After 10 min, 100 nmol of amino-modified DNA in 250 μL of 0.1 M NaHCO₃ was added to the solution and shaken at room temperature for 2 h. After 2 h, the reaction mixture was run through a NAP™-10 (GE Healthcare) column to separate away any free dye. To separate DNA sequences with and without the dye modification, RP-HPLC was then run on a C₁₈ column (0 to 75% B, 45 min, A=triethylammonium acetate buffer, B=acetonitrile).

### Synthesis, Purification, and Characterization of I-Motif and Control Gold NFS

In a typical reaction, 2 mL of 13 nm gold NPs were added to a 15 mL falcon tube and supplemented with Tween 20 to a final concentration of 0.2%. 10×PBS was added to the falcon tube such that its final concentration became 1×. The mixture was vortexed for 30 s and then sonicated for 30 s.

In a separate tube, recognition and flare strands were mixed in a 1:1 molar ratio and adjusted to 50 μM concentration in 1× duplexing buffer (30 mM HEPES, 100 mM KOAc, 2 mM MgOAc). This mixture was heated at 95° C. for 5 min and then allowed to cool to room temperature. The duplex was then added to the AuNP mixture at 300 equiv. duplex per AuNP. The subsequent mixture was allowed to incubate for 2 h, at which point 5 M NaCl was added to make the NaCl concentration 350 mM. After 1 h of further incubation, more 5 M NaCl was added to make the final NaCl concentration 500 mM. The mixture was then shaken for 48 h. After 48 h, the gold NFs were purified using 5 rounds of centrifugation (15000 rcf, 10 min) through successive pelleting and resuspension steps in 1×PBS.

The concentration of the gold NFs was determined via UV-vis spectroscopy using an extinction coefficient of 2.7×10⁸ M⁻¹cm⁻¹ for 13 nm gold nanoparticles [Halo, T. L.; McMahon, K. M.; Angeloni, N. L.; Xu, Y.; Wang, W.; Chinen, A. B.; Malin, D.; Strekalova, E.; Cryns, V. L.; Cheng, C.; Mirkin, C. A.; Thaxton, C. S NanoFlares for the Detection, Isolation, and Culture of Live Tumor Cells from Human Blood. Proc. Natl. Acad. Sci. U.S.A 2014, 111 (48), 17104-17109]. Next, the number of DNA strands per particle was determined. 40 μL of 50 nM gold NFs was added to 140 uL 8 M urea. 180 μL of 40 mM KCN was added to this mixture. Over the next 5-10 min, the gold nanoparticles were etched completely by KCN and this process could be followed visually as the wine-red color of the nanoparticles disappeared. The solution was heated to 60° C. for 10 min. The fluorescence of the resultant solution was measured using a plate reader both in the Cy3 (excitation: 554 nm, emission 600 nm) and Cy5 (excitation: 647 nm, emission: 700 nm) channels. The concentration of DNA could be calculated from a calibration curve of the Cy3- and Cy5-labeled recognition and flare strands in the same solvent mixture. All fluorescence measurements were performed in triplicate. The ratio of the concentration of DNA to that of the nanoparticles yielded the number of strands per particle. On average, each gold nanoparticle had approximately 40 duplexes (one flare strand for every recognition strand).

### Synthesis of ProSNAs

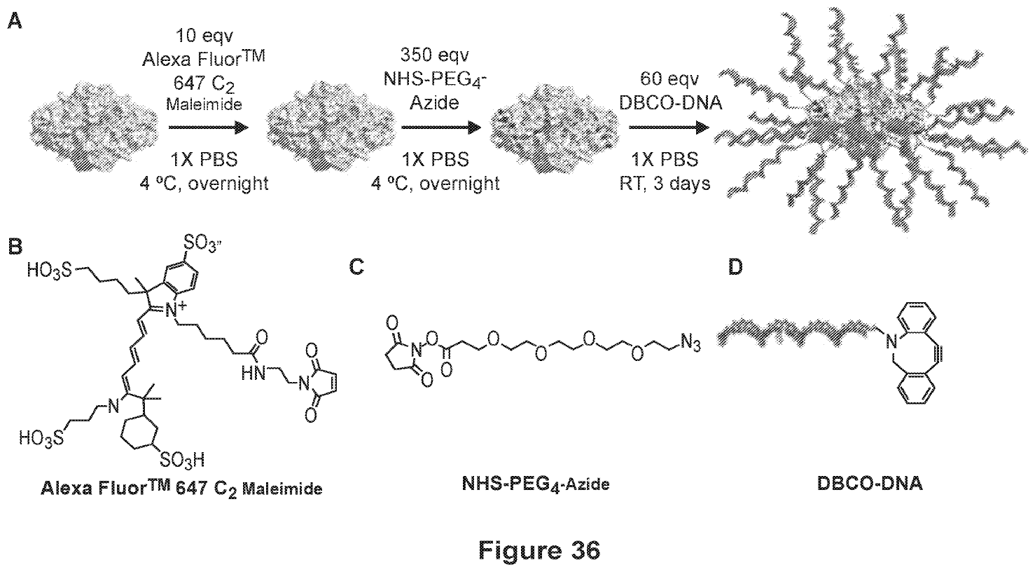

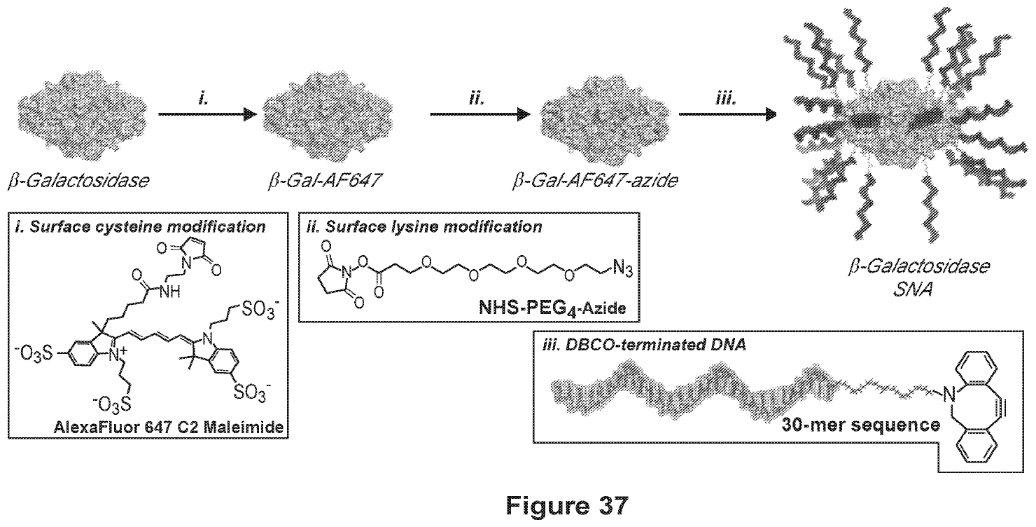

A general scheme for the synthesis of ProSNAs is shown in FIGS. 36 and 37.

### Synthesis, Purification, and Characterization of I-Motif and Control ProSNAs

β-galactosidase (β-gal) ProSNAs were synthesized and characterized following previously reported procedures [Kusmierz, C. D.; Bujold, K. E.; Callmann, C. E.; Mirkin, C. A. Defining the Design Parameters for in Vivo Enzyme Delivery Through Protein Spherical Nucleic Acids. ACS Cent. Sci. 2020, 6 (5), 815-822; Brodin, J. D.; Sprangers, A. J.; McMillan, J. R.; Mirkin, C. A. DNA-Mediated Cellular Delivery of Functional Enzymes. J. Am. Chem. Soc. 2015, 137 (47), 14838-14841]. Lyophilized β-gal from an E. coli overproducer (Roche) was centrifuged and resuspended in 1×PBS three times using a 100 kDa MWCO Amicon® filter to remove storage salts. Next, a thiol-reactive Alexa Fluor™ 647 C₂ Maleimide (ThermoFisher), AF-647, was introduced at a ten-fold excess, and the reaction was allowed to proceed overnight at 4° C. in 1×PBS with shaking. Multiple washing cycles were conducted in a 100 kDa MWCO Amicon® filter to remove the unreacted dye, resuspending β-Gal-AF-647 in 1×PBS after each centrifuge. Wash cycles were stopped once the filtrate did not have a detectable absorbance signal at approximately 653 nm, as monitored by a Cary-500 UV-vis spectrophotometer. A 350-fold excess of NHS-PEG₄-Azide (ThermoFisher) was added to β-Gal-AF-647 and incubated overnight at 4° C. in 1×PBS with shaking. Unreacted linker was removed by ten wash cycles using a 100 kDa MWCO Amicon® filter, and resuspending the β-Gal-AF-647-azide in 1×PBS after each centrifugation. The number of AF-647 modifications made to the protein were calculated based on absorbance spectra collected on a Cary-500 UV-vis spectrophotometer and their respective extinction coefficients (ε_{β-gal}=1,142,000 M⁻¹cm⁻¹ at 280 nm and 596,268 M⁻¹cm⁻¹ at 260 nm; ε_{AF-647}=270,000 at 650 nm). The number of PEG₄-azide linker modifications was assessed by MALDI-TOF MS using sinapinic acid (ThermoFisher) as a matrix in a Bruker AutoFlex-Ill. Each linker addition leads to an increase of 275 m/z. In a typical reaction, 60 molar equivalents (DNA:protein) of DBCO-modified DNA was added to a 1.5 mL Eppendorf tube and dried on a Centrivap. Protein (1×PBS, concentration approximately 6 μM β-gal) was then added to this DNA and shaken for 3 days (60 eq DNA:protein). After 3 days, the ProSNAs were purified through approximately 15-20 washes (1×PBS) using a 100 kDa Amicon filter. The number of DNA strands per protein was calculated based on UV-vis spectroscopy (ε_{i-motif DNA}=337,600 M⁻¹cm⁻¹ at 260 nm and ε_{control DNA}=315,400 M⁻¹cm⁻¹ at 260 nm). Because the DNA's max absorbance (260 nm) overlaps with the protein's max absorbance (280 nm), the AF-647 UV-vis trace was used to back-calculate the protein's concentration. There were approximately 6.8 AF-647 dyes per protein. Approximately 30 DNA strands were attached per i-motif ProSNA (ProTOn) and approximately 40 DNA strands per control ProSNA.

### Design, Synthesis, Purification, and Characterization of GOx-SNAs

Due to the absence of chemically accessible cysteine residues on the surface of glucose oxidase (GOx), both AF-647 and DNA were conjugated to the surface through lysine residues. The relative ratios of the two modifications were controlled by controlling the molar equivalents and the conjugation reaction time. On average, each protein contained approximately 2 AF-647 dyes and approximately 28 DNA strands. A simple DBCO-labeled T₁₃ sequence was used as the DNA shell to enable probe uptake and as in the case of β-gal, the AF-647 dyes allow for monitoring of probe uptake.

Glucose oxidase protein (Millipore-Sigma G7141) was first dissolved in 0.1 M NaHCO₃ at 10 mg/mL. Alexa Fluor™ 647 NHS Ester (Thermo-Fisher A37573) was dissolved at a concentration 10 mg/mL in DMF. This dye solution was added dropwise to the protein solution while vortexing in 13-fold molar excess. After shaking the mixture for 1 h at 1400 rpm, the unfunctionalized AF-647 was separated from the protein using a 30 kDa Amicon filter (13,000 rcf at 4° C.) and washing 10 times using 0.1 M NaHCO₃.

The protein was then labeled with PEG-azide to allow subsequent functionalization to DBCO-modified DNA. 300 μL of 50 μM protein in PBS was reacted with 18 μL NHS-PEG₄-Azide for 1.75 h. The mixture was then purified using a 30 kDa Amicon filter by washing with 1×PBS (10 washes, 13,000 rcf at 4° C.).

In a typical DNA functionalization reaction, 300 molar equivalents (DNA:protein) of DBCO-modified DNA was added to a 1.5 mL Eppendorf tube and dried on a Centrivap. Protein (1×PBS, concentration approximately 10 μM) was then added to this DNA and shaken for 3 days (300 eq DNA:protein). After 3 days, GOx-SNAs were purified through approximately 15-20 washes (1×PBS) using a 100 kDa Amicon filter.

The number of dyes and DNA strands per protein was calculated via UV-vis spectroscopy using the extinction coefficient of the DNA (ε_{DNA}=113,900 M−1 cm−1), protein (ε_{GOx}=267,200 M⁻¹cm⁻¹), and the dye (ε_{AF-647}=270,000 M⁻¹cm⁻¹).

### Fluorescence Experiments

All experiments were done in triplicate unless otherwise stated.

### Experiments with i-Motif and Control Gold NFs

Fluorescence melt to determine duplex melting temperature. The melting temperature of the flare/recognition strand duplex was determined by fluorescence melt experiments. This was done to ensure that the i-motif recognition/flare and control recognition/flare have comparable melting temperatures. Approximately 3 nM by gold i-motif or control NFs were added to pH 7.5 clamping buffer (Thermo-Fisher P35379). The temperature of a BioTek Cytation 5 fluorescence plate reader was set to x° C. (where x=28, 29, 31, 33, 35, 37, 39, 41, 43, 45, 47, 49, 51, 53, 55, 57, 59, 61, 63, 65) and samples were shaken for 5 min. After 5 min, a fluorescence reading was taken, acquiring a fluorescence reading at each temperature (excitation: 554 nm, emission: 600 nm for the Cy3 dye; excitation: 647 nm, emission: 700 nm for the Cy5 dye).

pH sensitivity of i-motif gold NFs in buffer. i-motif or control NFs were incubated in clamping buffer of varying pH to assess the response of the constructs to pH. The pH of buffer was adjusted using NaOH. 1.7 nM by gold i-motif or control NF was added to buffer of pH 4.5 5.0, 5.5, 6.0, 6.5, 7.0, and 7.5 and incubated for 30 min at 37° C. A BioTek Cytation 5 fluorescence plate reader (excitation 554 nm, emission 600 nm for the Cy3 dye, excitation 647 nm, emission 690 nm for the Cy5 dye) was used to measure the fluorescence at each pH.

Fluorescence response of gold NFs in presence of nuclease in buffer. In triplicate, 2 nM gold NFs (by gold) was added to 100 μL 1×DNAse buffer and allowed to incubate at 37° C. for 30 min. After 30 min, 10 μL of 0.2 U/μL DNAse I (Themo-Fisher AM2224) was added to each well (10 μL of water was added to wells not treated with DNAse as a DNAse free control). Fluorescence was monitored on a BioTek Cytation 5 plate reader (excitation 554 nm, emission 600 nm for the Cy3 dye, excitation 647 nm, emission 690 nm for the Cy5 dye) every 1 min over the course of 15 min.

Change of fluorescence signal over time of gold NFs in cellulo. MDA-MB-231 cells were treated in a 24 well plate with 1 nM (by gold) control NFs in Opti-MEM (Thermo-Fisher 31985062). All cells were pulsed for 1 h and chased for either 0, 2, or 4 h in triplicate. Cells were washed with 400 μL Opti-MEM before the pulsing step and 400 μL Opti-MEM before the chasing step. After the chasing step, cells were detached from the plate using 1×TrypLE (Thermo-Fisher 12604021) with added 4′,6-diamidino-2-phenylindole (DAPI) and then analyzed using flow cytometry (BD LSRFortessa and BD Symphony A3).

### Experiments with i-Motif and Control ProSNAs

pH sensitivity of ProTOn in buffer. i-motif or control ProSNAs were incubated in clamping buffer of varying pH to assess the response of the constructs to pH. 500 nM (by DNA) of ProTOn or control ProSNA were added to buffer of pH 5.0, 5.5, 6.0, 6.5, 7.0, and 7.5 in triplicate at room temperature. A BioTek Cytation 5 fluorescence plate reader (excitation 485 nm, emission 528 nm for the thiazole orange dye, excitation 647 nm, emission 690 nm for the AF-647 dye) was used to measure the fluorescence at each pH.

Fluorescence response of ProSNAs in presence of nuclease in buffer. In triplicate, 500 nM (by DNA) of i-motif or control ProSNA was added to 100 μL 1×DNAse buffer and incubated at 37° C. for 15 min. 10 μL of 0.2 U/μL DNAse I was added to induce nuclease degradation. 10 μL of water was added to the wells not treated with DNAse I as a control. The fluorescence was monitored on a BioTek Cytation 5 plate reader (excitation 485 nm, emission 528 nm for the thiazole orange dye) every 1 min over the course of 15 min.

Response of ProSNAs to proteases in buffer. For this specific study, the ProSNA used was comprised of β-gal densely functionalized with a DBCO-dT-(sp18)₂T₃₀ sequence (approximately 34 DNA per protein). The native protein and the ProSNA were incubated with 250 mg/L Trypsin (Gibco) in 1×PBS at 37° C. with shaking. As the degradation reaction proceeded, aliquots were removed every 10 min for a total of 70 min and loaded onto a 7.5% Mini-PROTEAN TGX precast gel (BioRad). The samples were loaded onto the gel using Laemmli sample buffer (BioRad) and the gel was run at 100V for 1.5 h in TGS running buffer. The protein bands were visualized by staining using a SimplyBlue SafeStain (Thermo Fisher).

Change of fluorescence signal over time of ProSNAs in cellulo. MDA-MB-231 cells were treated in a 24 well plate with 500 nM (by DNA) control ProSNAs. All cells were pulsed for 1 h and chased for either 0, 2, or 4 h in triplicate. Cells were washed with 400 μL Opti-MEM before the pulsing step and 400 μL Opti-MEM before the chasing step. After the chasing step, cells were detached from the plate using 1×TrypLE containing DAPI and then analyzed using flow cytometry.

Fluorescence response of i-motif ProSNA after clamping cellular pH. MDA-MB-231 cells were treated in a 24 well plate with 500 nM (by DNA) of ProTOn or control ProSNA. Cells were treated for 3 h, after which they were washed once with 400 μL Opti-MEM and subsequently detached using 1×TrypLE. Each well of the plate was then pipetted into separate Eppendorf tubes, after which tubes were centrifuged to pellet the cells. The supernatant was aspirated off, and cells were suspended in pH 5.5 or pH 7.5 clamping buffer for 10 min before being analyzed by flow cytometry. Control cells not treated with SNAs were also analyzed to ensure that different pH's do not alter the autofluorescence of the cells. As a control, commercially available pHrodo™ Red AM Intracellular pH Indicator (Thermo Fisher P35372) was also used to measure the fluorescence difference between cells clamped at pH 5.5 and pH 7.5. Cells were treated according to manufacturer protocol with no modification.

### Experiments with GOx-SNAs

Fluorescence response of GOx-SNAs to glucose in buffer. Varying concentrations of glucose (in 1×PBS) were added to the wells of a 96 well plate, whereby the concentration of glucose was halved in each well through a half serial dilution (a separate set of wells were prepared that had 0 mM glucose). All wells contained 5 μM FBBBE (Cayman Chemical 14606). The plate was then allowed to incubate for 30 min at 37° C. After 30 min, an automatic dispensing unit on a BioTek Cytation 5 fluorescence plate reader was used to add GOx-SNAs to each well at a final concentration of 20 nM by protein (a buffer only set of wells was also prepared). After addition, the BioTek Cytation 5 plate reader was used to shake the sample for 15 s, and subsequently a fluorescence reading was taken every 3 min over 2 h (excitation 460 nm, emission 530 nm for FBBBE, excitation 640 nm, emission 700 nm for the Alexa Fluor 647 dye).

A separate control experiment was done to ensure that the dye fluorescence in the absence of GOx-SNAs does not change with increasing glucose concentration. Varying concentrations of glucose (in 1×PBS) were added to the wells of a 96 well plate, whereby the concentration of glucose was halved in each well through a half serial dilution (a separate set of wells were prepared that had 0 mM glucose). All wells contained 5 μM FBBBE. The samples were incubated at 37° C. for 30 min, after which a fluorescence reading was taken every 3 min over 2 h (excitation 460 nm, emission 530 nm for FBBBE).

GOx-SNAs fluorescence response to “off-target” sugars in buffer. In triplicate, 20 nM (by protein) GOx-SNAs was co-incubated with 5 μM FBBBE and one different sugar (either 5 mM glucose, sucrose, xylose, mannose, glucose 6-phosphate, fructose, maltose, lactose, or galactose) in 1×PBS. Controls for GOx-SNAs+FBBBE, FBBBE only, GOx-SNAs only, and 1×PBS only were also done. Samples were incubated at 37° C. for 30 min after which a fluorescence reading was taken on a BioTek Cytation 5 plate reader (excitation 460 nm, emission 530 nm for the FBBBE dye).

In another experiment, 10 nM (by protein) GOx-SNAs were incubated with 5 μM FBBBE and all the sugars (5 mM glucose, sucrose, xylose, mannose, glucose 6-phosphate, fructose, maltose, lactose, and galactose) in 1×PBS. Controls for GOx-SNAs+FBBBE+5 mM glucose, GOx-SNAs+FBBBE, FBBBE only, GOx-SNAs only, and 1×PBS only were also done. Samples were incubated at 37° C. for 30 min after which a fluorescence reading was taken on a BioTek Cytation 5 plate reader (excitation 485 nm, emission 528 nm for the FBBBE dye).

GOx-SNAs activity versus native protein. 20 nM native glucose oxidase protein or 20 nM GOx-SNAs were added to 1×PBS in the presence of 1 mM glucose and 5 μM FBBBE at 37° C. in triplicate. A reading of fluorescence was taken every 3 min over 2 h on a BioTek Cytation 5 (excitation 485 nm, emission 528 nm for the FBBBE dye, excitation 647 nm, emission 690 nm for the Alexa Fluor 647 dye).

Fluorescence response of GOx-SNAs in different cell lines. Nine different cell lines (MDA-MB-231, MC38, U87, SKOV3, HDF, EL4, EG7-OVA, 4T1, and NIH/3T3) were tested, representing a mix of cancer and normal cells, adherent and suspension cells, and human and murine-derived cells. Adherent cells were detached from culture dish using 1×TrypLE and subsequently pelleted by centrifugation. The supernatant was removed and the cells were washed twice with 6 mL glucose-free DMEM (Thermo-Fisher 11966025) through successive pelleting and resuspension steps. After the second wash, the cell suspension was split into 3 different treatment groups, each run in triplicate. In two of the treatment groups, the cells were suspended in glucose-free media only and in the third treatment group the cells were suspended in glucose-free media containing 40 nM GOx-SNAs (by protein). After 30 min at 37° C., cells were pelleted by centrifugation, the supernatant was removed, and all cells were washed twice with 1 mL glucose-free media through successive pelleting and resuspension steps. In the first treatment group, previously untreated cells were resuspended in DMEM supplemented with 25 mM glucose (untreated group). In the second treatment group, previously untreated cells were resuspended in DMEM supplemented with 25 mM glucose and 50 μM FBBBE (dye only group). In the third treatment group, previously SNA treated cells were resuspended in DMEM supplemented with 25 mM glucose and 50 μM FBBBE (dye+SNA group). After 30 min at 37° C., cells were pelleted by centrifugation, resuspended in 1×TrypLE containing DAPI, and analyzed using flow cytometry. This procedure was the same for all adherent cell lines.

Suspension cells were pelleted by centrifugation and washed twice with 6 mL glucose-free DMEM through successive pelleting and resuspension steps. After the second wash, the cell suspension was split into 3 different treatment groups each run in triplicate. In two of the treatment groups, cells were suspended in glucose-free media only and in the third treatment group the cells were suspended in glucose-free media containing 40 nM GOx-ProSNA (by protein). After 30 min at 37° C., cells were pelleted by centrifugation, the supernatant was removed, and all cells were washed twice with 1 mL glucose-free media through successive pelleting and resuspension steps. In the first treatment group, previously untreated cells were resuspended in DMEM supplemented with 25 mM glucose (untreated group). In the second treatment group, previously untreated cells were resuspended in DMEM supplemented with 25 mM glucose and 50 μM FBBBE (dye only group). In the third treatment group, previously SNA treated cells were resuspended in DMEM supplemented with 25 mM glucose and 50 μM FBBBE (dye+SNA group). After 30 min at 37° C., cells were pelleted by centrifugation, resuspended in 1×TrypLE containing DAPI, and analyzed using flow cytometry. This procedure was the same for all suspension cell lines.

Fluorescence response of GOx-SNAs in cells exposed to varying glucose concentrations. EL4 suspension cells were pelleted by centrifugation and washed twice with 6 mL glucose-free DMEM through successive pelleting and resuspension steps. After the second wash, the cell suspension was split into different treatment groups, each run in triplicate. The cells were suspended in glucose-free media containing 40 nM GOx-SNAs (by protein). After 30 min at 37° C., cells were pelleted by centrifugation, the supernatant was removed, and all cells were washed twice with 1 mL glucose-free media through successive pelleting and resuspension steps. The cells were then resuspended in DMEM containing 50 μM FBBBE with 0 or 25 mM glucose. After 30 min at 37° C., cells were pelleted by centrifugation, resuspended in TrypLE containing DAPI, and analyzed using flow cytometry. Relative glucose levels in cells measured by flow cytometry were compared with glucose levels measured in cell lysates using a commercially available glucose assay kit, as described herein below.

Fluorescence response of GOx-SNAs in cells when glucose uptake increases. The insulin-sensitive MC38 cell line was used in this study. MC38 cells were detached from culture dish using 1×TrypLE and subsequently pelleted by centrifugation. The supernatant was removed and the cells were washed twice with 6 mL glucose-free DMEM (Thermo-Fisher #11966025) through successive pelleting and resuspension steps. After the second wash, the cell suspension was split into different treatment groups, each run in triplicate. The cells were suspended in glucose-free media containing 40 nM GOx-SNAs (by protein). After 30 min at 37° C., cells were pelleted by centrifugation, the supernatant was removed, and all cells were washed twice with 1 mL glucose-free media through successive pelleting and resuspension steps. The cells were resuspended in DMEM containing 5 mM glucose and 50 μM FBBBE with 0 or 100 nM insulin from Thermo Fisher catalog #12585014. After 30 min at 37° C., cells were pelleted by centrifugation, resuspended in TrypLE containing DAPI, and analyzed using flow cytometry.

Fluorescence response of GOx-SNAs in cells when glucose uptake is inhibited. EL4 suspension cells were pelleted by centrifugation and washed twice with 6 mL glucose-free DMEM through successive pelleting and resuspension steps. After the second wash, the cell suspension was split into different treatment groups, each run in triplicate. The cells were suspended in glucose-free media containing 40 nM GOx-SNAs (by protein). After 30 min at 37° C., cells were pelleted by centrifugation, the supernatant was removed, and all cells were washed twice with 1 mL glucose-free media through successive pelleting and resuspension steps. The cells were resuspended in DMEM containing 25 mM glucose and 50 μM FBBBE with 0 or 10 μM cytochalasin B from Millipore Sigma catalog #C6762. After 30 min at 37° C., cells were pelleted by centrifugation, resuspended in TrypLE containing DAPI, and analyzed using flow cytometry. Relative glucose levels in cells measured by flow cytometry were compared with glucose levels measured in cell lysates using a commercially available glucose assay kit, as described herein below.

Measurement of glucose in cell lysate. EL4 cells were pelleted by centrifugation and washed twice with 6 mL glucose-free DMEM through successive pelleting and resuspension steps. After the second wash, the cell suspension was split into 3 different treatment groups, each run in triplicate. In all of the treatment groups, cells were first suspended in glucose-free media. After 30 min at 37° C., cells were pelleted by centrifugation, the supernatant was removed, and then cells were treated in 3 groups. In the first treatment group, cells were resuspended in DMEM containing no glucose (0 mM glucose treatment group). In the second treatment group, cells were resuspended in DMEM supplemented with 25 mM glucose and 10 μM cytochalasin B (glucose inhibitor group). In the third treatment group, cells were resuspended in DMEM supplemented with 25 mM glucose (25 mM glucose treatment group). After 30 min at 37° C., cells were combined and pelleted by centrifugation, the supernatant was removed, and cells were washed twice with 6 mL DPBS through successive pelleting and resuspension steps. After the second wash, cells were resuspended in 1 mL of glucose assay buffer (Abcam 169559). Cells were then lysed through 5 freeze-thaw cycles.

After lysis, cells were centrifuged to remove any cellular debris and the supernatant was collected for further analysis. A glucose assay kit (Abcam 169559) was used to measure relative glucose concentrations between the 3 different conditions (150,000 cells analyzed in each well). Lysate was first filtered through a 10 kDa Amicon filter to remove any interfering proteins, and then assayed according to the manufacturer protocol using a BioTek Cytation 5 fluorescence plate reader.

### Data Analysis and Statistics

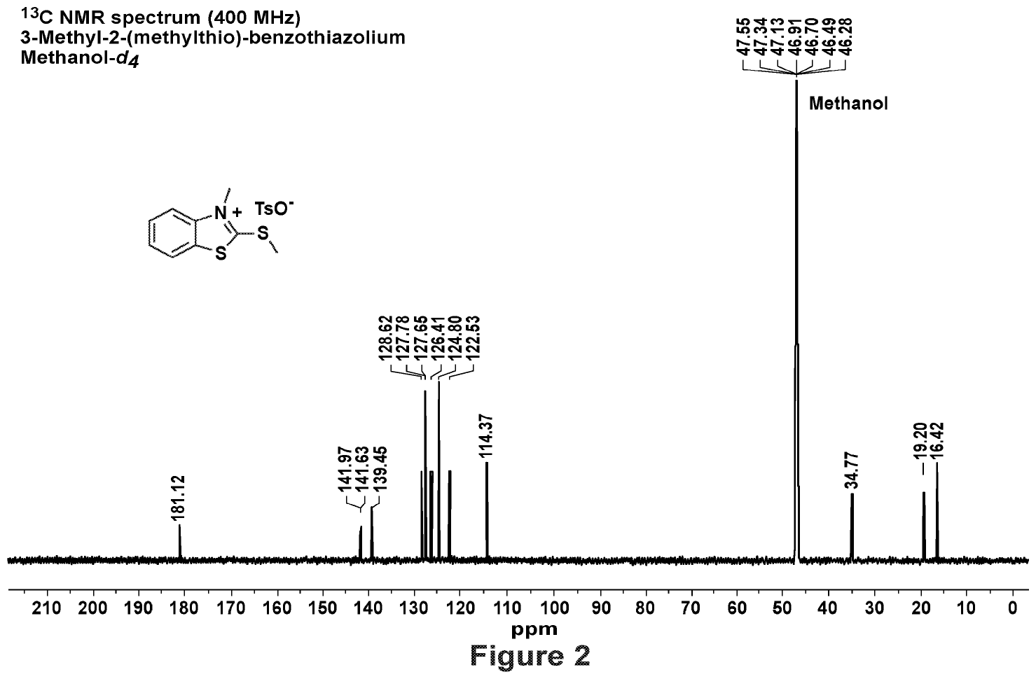

The figures herein show the mean of three independent readings unless otherwise mentioned. Error bars correspond to one standard deviation. For FIG. 2B, the p-value was determined via a t-test using a one-tailed hypothesis (this is because fluorescence at pH 5.5 should be higher than fluorescence at pH 7.5).

### I-Motif and Control Gold NFS

### Fluorescence Melt to Determine the Melting Temperature of Flare Strands

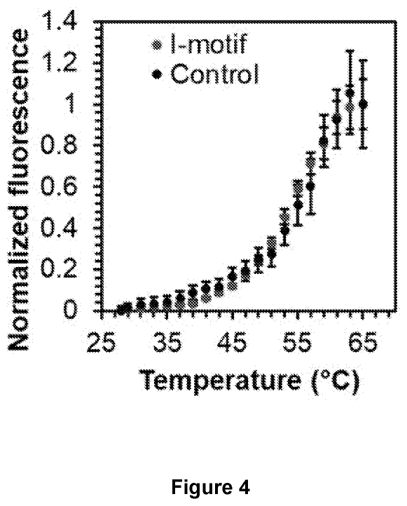

For a fair comparison of the false-positive signal obtained from gold NFs, it is important to ascertain that the flares strands bind to the recognition sequences with similar affinity. As a proxy for the binding affinity, the melting behavior of the duplexes was investigated. When the flare strand is hybridized to the recognition sequence, its proximity to the gold nanoparticle quenches its fluorescence. However, as the temperature of the sample is gradually increased, DNA dehybridization occurs. Therefore, the fluorescence of the flare sequence gradually increases as it is separated from the gold nanoparticle. From the figure below (FIG. 4), the melting temperatures of the flare strands were observed to be nearly identical for both the i-motif and control gold NFs.

### pH-Sensitivity of i-Motif Gold NFs in Buffer

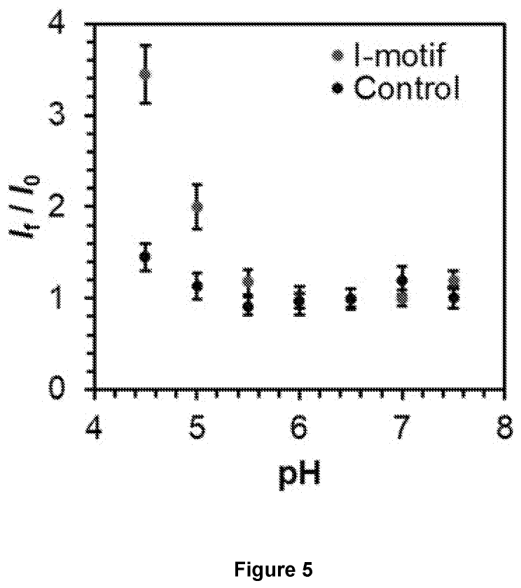

Approximately 1.7 nM of i-motif and control gold NFs were subjected to different pH between 7.5 and 4.5 at 37° C. and their fluorescence was monitored. The fluorescence of i-motif gold NFs increases by up to approximately 3.5-fold as the pH decreases due to the formation of i-motif structures and displacement of the flare sequence. In contrast, the fluorescence of the pH-insensitive control NF does not change significantly in this pH range. FIG. 5.

### Fluorescence Response of Gold NFs in the Presence of Nucleases in Buffer

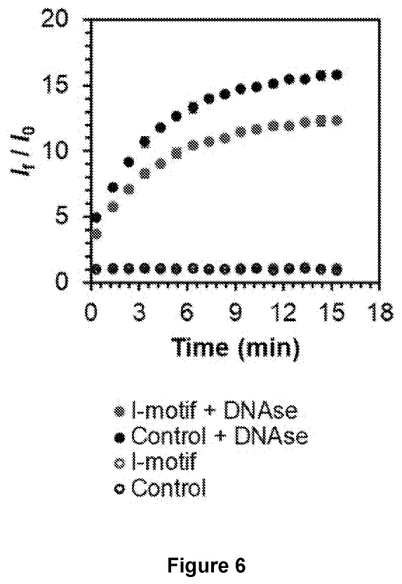

To investigate the effect of nuclease degradation on gold NFs, 2 nM gold NFs were incubated with 2 U DNAse I for at 37° C. In the presence of DNAse I, the fluorescence increases over time for both the i-motif and the control sequence. In contrast, in the absence of DNAse I, the fluorescence remains unchanged. These results showed that nuclease degradation leads to over 15-fold fluorescence enhancement in the absence of a recognition event, giving rise to false-positive signals. FIG. 6.

### Change in Fluorescence Signal Over Time of Control Gold NFs in Cellulo

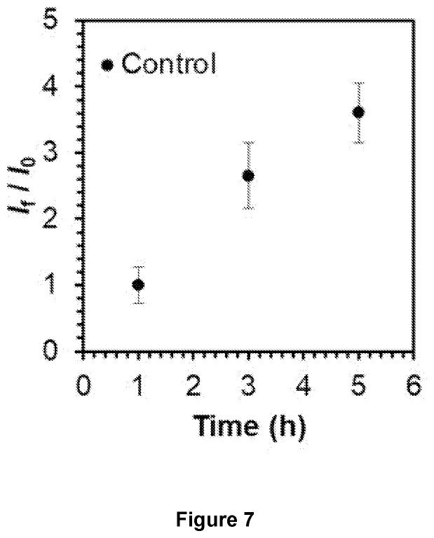

To determine the extent of false-positive signal obtained from NFs in cells, pulse-chase experiments were performed with the control gold NFs. 1 nM of the control NFs were incubated with MDA-MB-231 cells for 1 h in serum-free media. The cells were then washed thoroughly and after additional 0, 2, and 4 h, they were analyzed by flow cytometry. The fluorescence from the flare strand was monitored and found to increase over time. These results showed that a time-dependent false-positive fluorescence signal is observed from gold NFs in cells. FIG. 7.

### ProTOn and Control ProSNAs

### Fluorescence Response of ProSNAs in Presence of Nuclease in Buffer

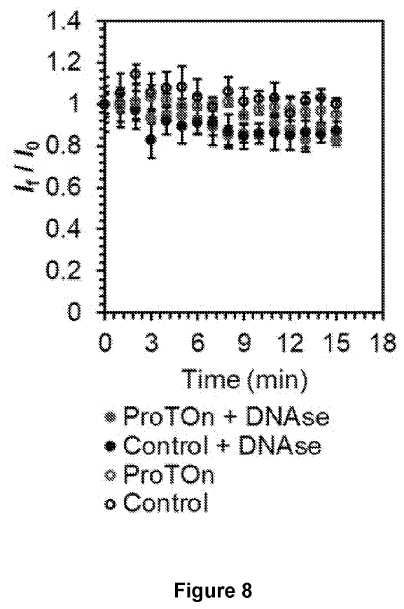

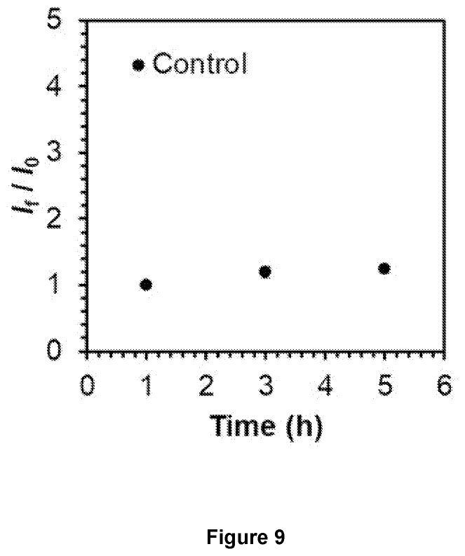

To investigate the effect of nuclease degradation on ProSNAs, 500 nM (by DNA) of ProTOn or control ProSNA were incubated with 2 U DNAse I for at 37° C. The fluorescence remained unchanged in both the presence and absence of DNAse I. These results showed that nuclease degradation does not give rise to a false-positive signal. FIG. 8. The change in fluorescence signal over time of control ProSNAs in cellulo is shown in FIG. 9.

### Fluorescence Response of pHrodo™ Red AM Intracellular pH Indicator

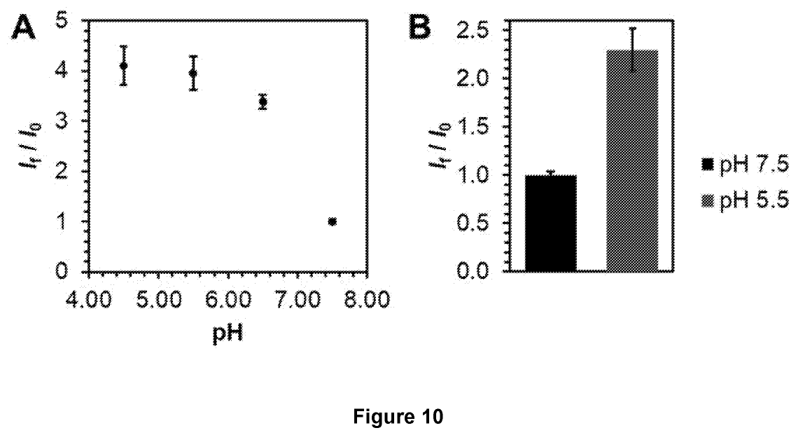

To benchmark ProTOn against a commercially available intracellular pH indicator, the pH response of pHrodo™ Red AM was studied. This hydrophobic pH-sensitive dye is cell permeable. In the cell, non-specific esterases cleave the AM ester groups and, consequently, the dye is retained intracellularly. The pH response of pHrodo™ Red AM was studied both in buffered solutions as well as in cells clamped to specific pH. See FIG. 10.

### The ProSNA Architecture Protects the Protein Against Protease Degradation

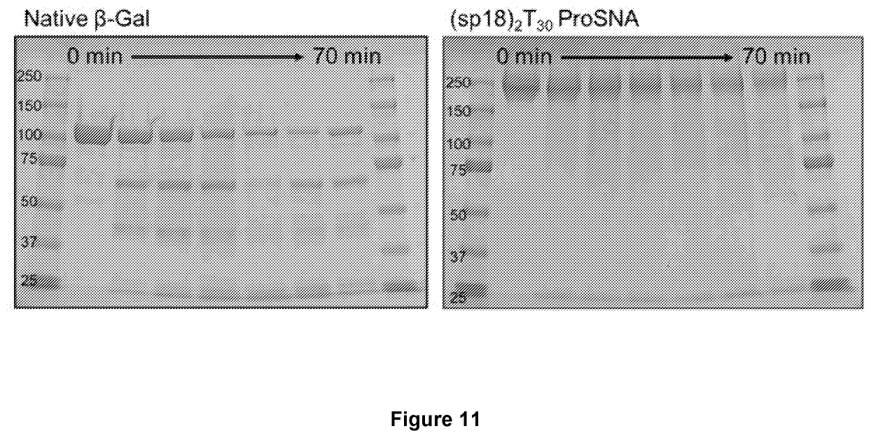

To demonstrate the effectiveness of the SNA architecture in protecting a protein from protease degradation, both the native protein and the ProSNA were incubated with a protease (trypsin) and aliquots of this degradation reaction were loaded on an SDS-PAGE gel. It was observed that the native protein incubated with trypsin produced several lower molecular weights corresponding to degradation products after 10 min. Furthermore, the intensity of these degradation products increased with time. However, with the ProSNA these degradation bands were not observed suggesting that the DNA shell is able to partially protect the protein from substantial degradation by trypsin. See FIG. 11.

### GOx-SNAS

### GOx-Characterization

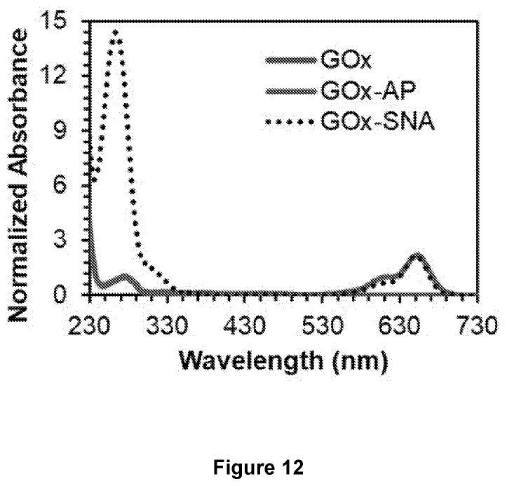

Since this is the first report of GOx-SNAs, UV-vis characterization data is provided herein. Native GOx had an absorbance peak at 280 nm. Upon modifying the GOx with AF-647 and PEG azides (i.e., for GOx-AP), an additional absorbance peak is observed at 650 nm corresponding to AF-647. Upon further functionalization with DNA (i.e., for GOx-SNAs), an additional peak is observed at 260 nm corresponding to the DNA. Based on the extinction coefficients of the GOx, AF-647, and DNA, the number of AF-647 dyes per GOx-SNA were calculated to be approximately 2 while the number of DNA strands was calculated to be approximately 28. See FIG. 12.

### Activity of GOx-SNAs Compared to Native GOx

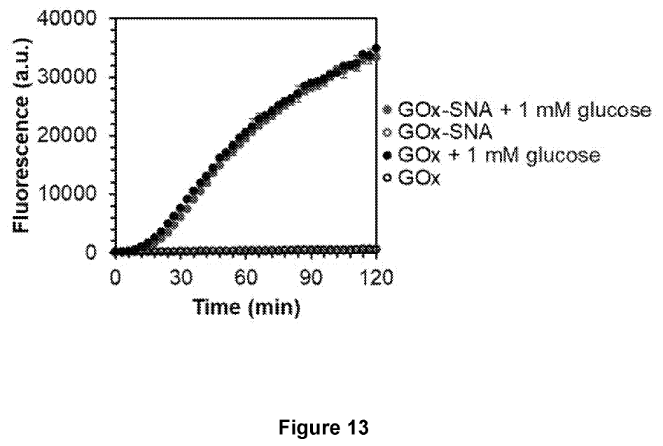

To determine the effect of conjugation of DNA to GOx on its catalytic activity, identical concentrations (20 nM) of GOx and GOx-SNAs were incubated with and without 1 mM glucose in 1×PBS containing 5 μM FBBBE at 37° C. GOx catalyzes the conversion of glucose to gluconic acid with concomitant production of hydrogen peroxide. The hydrogen peroxide formed reacts with FBBBE and cleaves the boronate ester groups, yielding highly fluorescent fluorescein. FIG. 13 shows that the fluorescence increases significantly over time in the presence of glucose. Importantly, the increase in fluorescence for both GOx and GOx-SNAs is nearly identical showing that the activity of GOx was retained in SNA form.

### Response of GOx-SNAs to Increasing Glucose Concentrations in Buffer

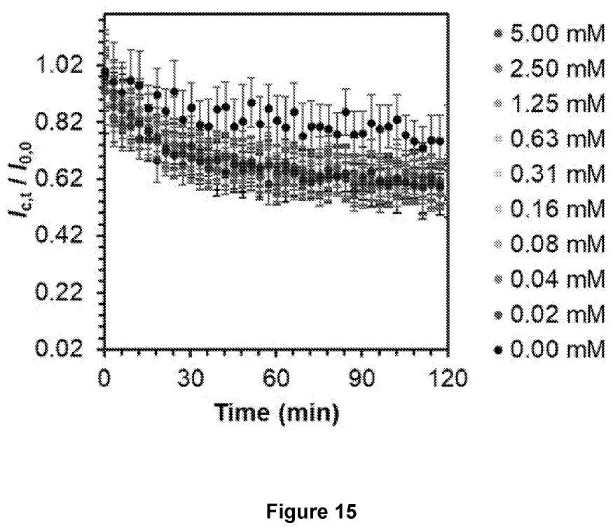

To determine the response of GOx-SNAs to increasing glucose concentrations in buffer, 20 nM GOx-SNAs was incubated with 5 μM FBBBE in 1×PBS at 37° C. and varying amounts of glucose between 0-5 mM was added. The fluorescence from FBBBE was monitored over a period of 2 h. A calibration curve was constructed at the 30 min and 2 h time points. The limit of detection (LOD) was determined at each time point by the 3δ/m method, where 8 denotes the standard deviation of the response and m denotes the initial slope of the calibration curve. The LODs were to be approximately 17 μM and approximately 5 μM at 30 min and 2 h, respectively. Both LODs are well below the typical concentration of glucose in cells (0.1-2 mM) [Nascimento, R. A. S.; Ozel, R. E.; Mak, W. H.; Mulato, M.; Singaram, B.; Pourmand, N. Single Cell “Glucose Nanosensor” Verifies Elevated Glucose Levels in Individual Cancer Cells. Nano Lett. 2016, 16 (2), 1194-1200; Cline, G. W.; Petersen, K. F.; Krssak, M.; Shen, J.; Hundal, R. S.; Trajanoski, Z.; Inzucchi, S.; Dresner, A.; Rothman, D. L.; Shulman, G. I. Impaired Glucose Transport as a Cause of Decreased Insulin-Stimulated Muscle Glycogen Synthesis in Type 2 Diabetes. N. Engl. J. Med. 1999, 341 (4), 240-246]. See FIG. 15

### Selectivity of GOx-SNAs for Glucose Over Other Sugars in a Complex Mixture

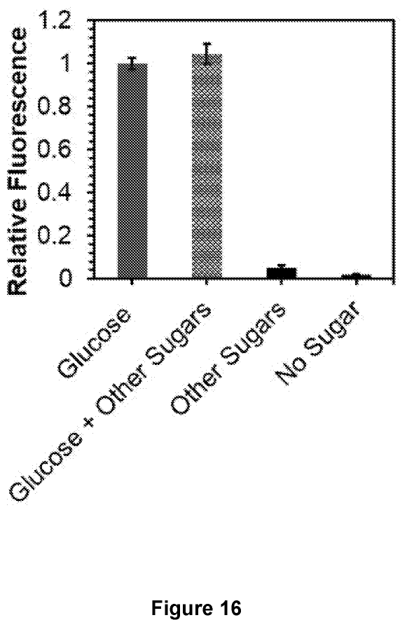

It was studied whether GOx-SNAs can selectively detect glucose in the presence of other sugars (sucrose, xylose, mannose, fructose, maltose, lactose, galactose, and glucose-6-phosphate). It was noted that the fluorescence observed in the presence of glucose was nearly identical to the fluorescence observed when glucose was present in a mixture of other sugars. Importantly, the fluorescence observed in the absence of glucose was negligible. See FIG. 16

### Response of GOx-SNAs in Different Cell Lines

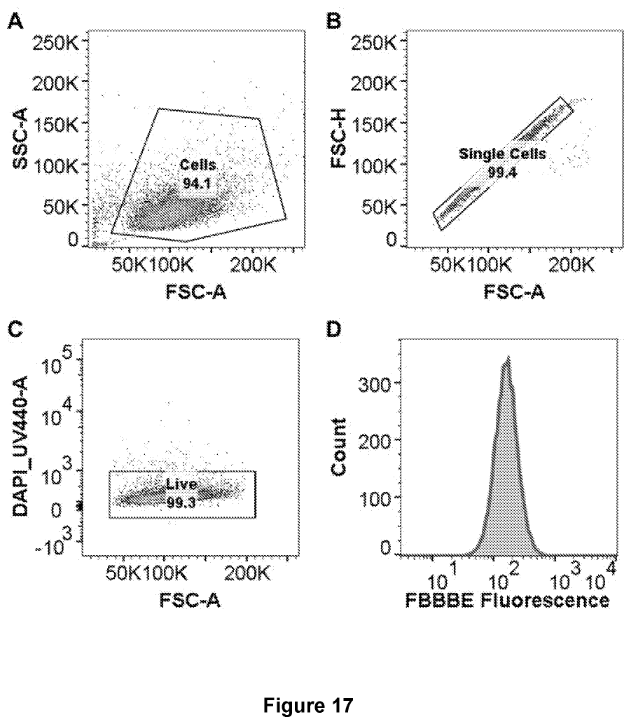

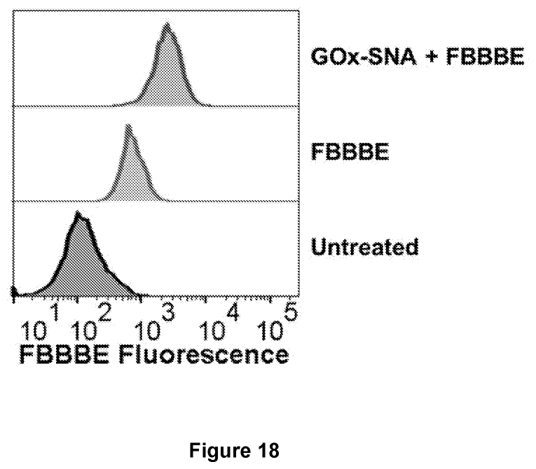

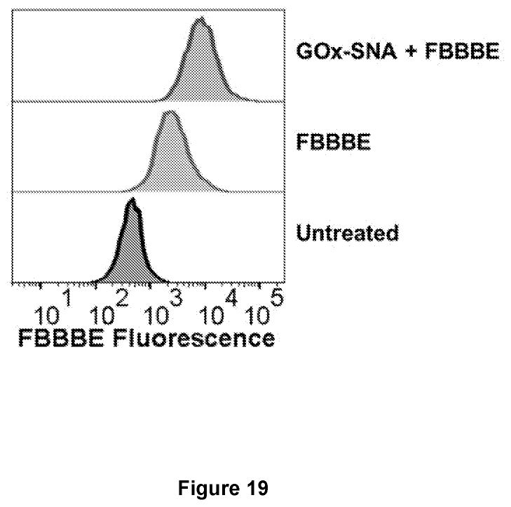

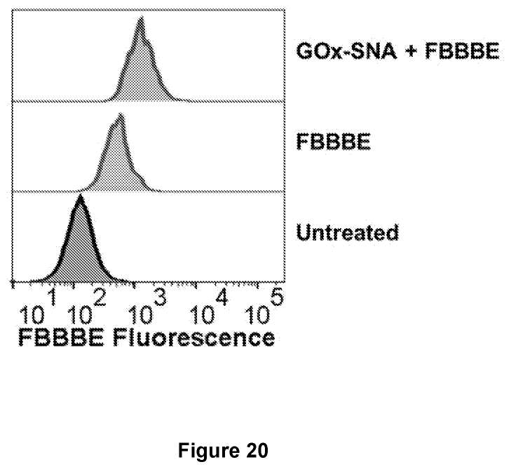

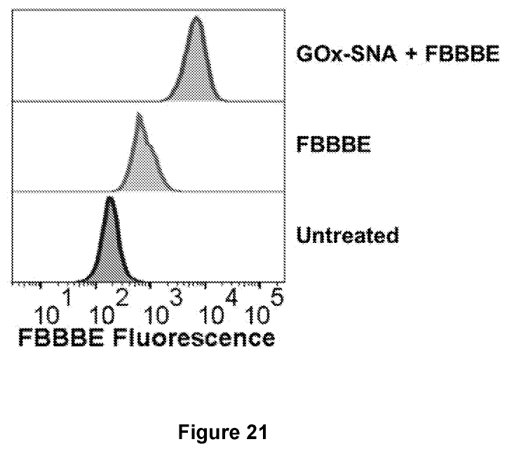

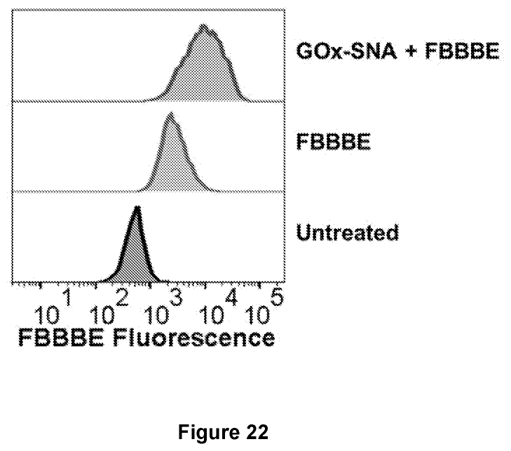

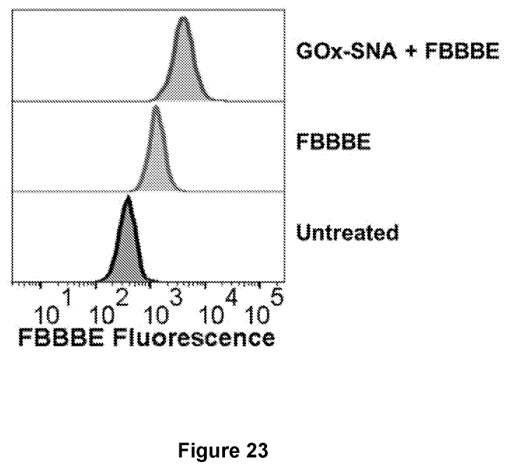

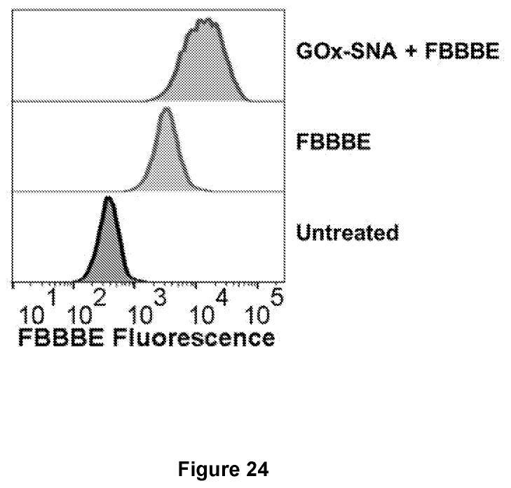

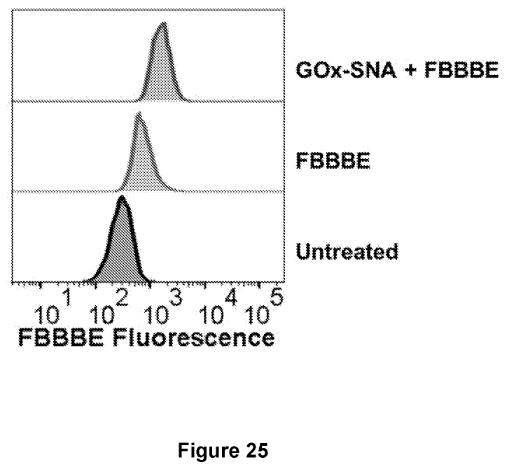

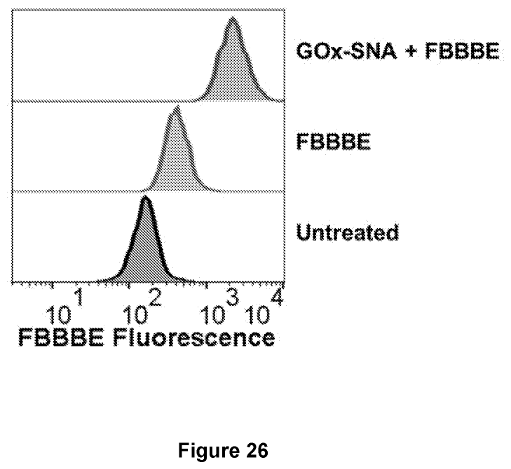

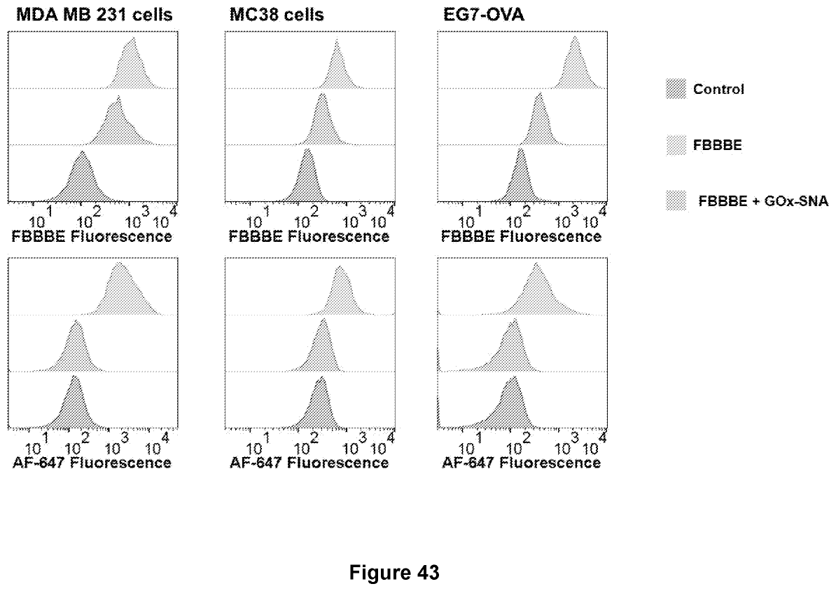

An example flow cytometry gating strategy is shown in FIG. 17. Glucose detection in MDA-MB-231 cells is shown in FIG. 18. Glucose detection in U87 cells is shown in FIGS. 19 and 43. Glucose detection in SKOV-3 cells is shown in FIG. 20. Glucose detection in EL4 cells is shown in FIG. 21. Glucose detection in Human Dermal Fibroblasts (HDF) cells is shown in FIG. 22. Glucose detection in MC38 cells is shown in FIG. 23. Glucose detection in NIH-3T3 cells is shown in FIGS. 24 and 43. Glucose detection in 4T1 cells is shown in FIG. 25. Glucose detection in EG7-OVA cells is shown in FIG. 26.

### Intracellular Response of GOx-SNAs to Varying Glucose Concentrations in Cell Culture Media

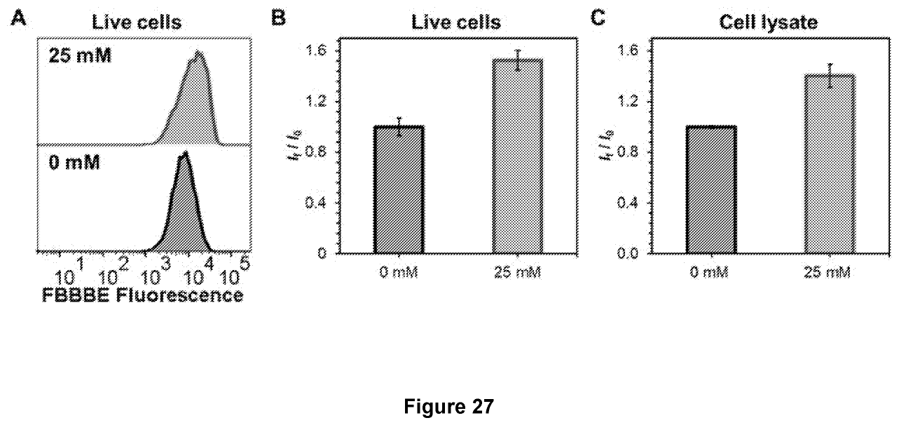

To study the effect of glucose added to cell culture media on the fluorescence reported by GOx-SNAs, EL4 cells were first treated with 40 nM GOx-SNAs in glucose-free media. After washing the cells thoroughly, 50 μM FBBBE in 0 or 25 mM glucose-containing media was added to the cells. The cells were incubated at 37° C. for 30 min and then analyzed by flow cytometry. It was noted that the fluorescence of cells subjected to 25 mM glucose-containing media was approximately 50% higher than cells incubated in 0 mM glucose. Importantly, these results agreed with the fluorescence observed from cell lysates when a commercially available glucose assay kit was used. See FIGS. 27 and 43.

### Intracellular Response of GOx-SNAs to Increased Glucose Uptake

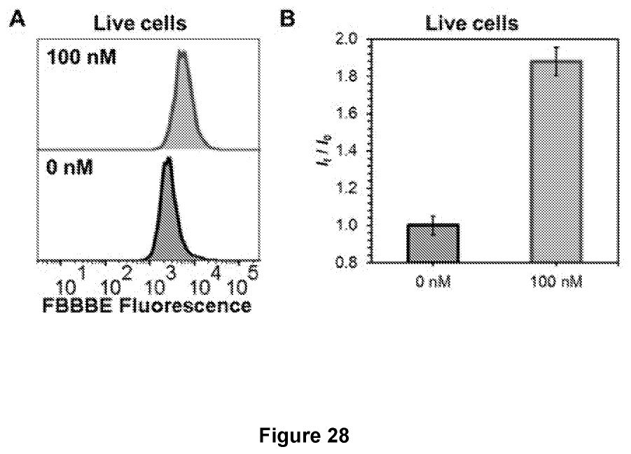

To study the effect of increased glucose uptake on the fluorescence reported by GOx-SNAs, MC38 cells were first treated with 40 nM GOx-SNAs in glucose-free media. After washing the cells thoroughly, 50 μM FBBBE in 5 mM glucose-containing media was added to the cells. Additionally, either 0 or 100 nM insulin, well-known to increase glucose uptake, was added [Rabin-Court, A.; Rodrigues, M. R.; Zhang, X.-M.; Perry, R. J. Obesity-Associated, but Not Obesity-Independent, Tumors Respond to Insulin by Increasing Mitochondrial Glucose Oxidation. PLoS One 2019, 14 (6), e0218126; Leto, D.; Saltiel, A. R. Regulation of Glucose Transport by Insulin: Traffic Control of GLUT4. Nat. Rev. Mol. Cell Biol. 2012, 13 (6), 383-396]. The cells were incubated at 37° C. for 30 min and then analyzed by flow cytometry. It was noted that the fluorescence of cells subjected to 100 nM insulin was increased by approximately 90% compared to cells not treated with the insulin. Importantly, these results agreed with previous reports of the effect of insulin on glucose uptake in MC38 cells measured using radiolabeled glucose [Rabin-Court, A.; Rodrigues, M. R.; Zhang, X.-M.; Perry, R. J. Obesity-Associated, but Not Obesity-Independent, Tumors Respond to Insulin by Increasing Mitochondrial Glucose Oxidation. PLoS One 2019, 14 (6), e0218126]. See FIG. 28.

### Intracellular Response of GOx-SNAs to Inhibition of Glucose Uptake

To study the effect of blocking glucose receptors on the fluorescence reported by GOx-SNAs, EL4 cells were first treated with 40 nM GOx-SNAs in glucose-free media. After washing the cells thoroughly, 50 μM FBBBE in 25 mM glucose-containing media was added to the cells. Additionally, either 0 or 10 μM cytochalasin B, a well-known glucose transport inhibitor, was added [Estensen, R. D.; Plagemann, P. G. W. Cytochalasin B: Inhibition of Glucose and Glucosamine Transport. Proc. Natl. Acad. Sci. 1972, 69 (6), 1430-1434]. The cells were incubated at 37° C. for 30 min and then analyzed by flow cytometry. It was noted that the fluorescence of cells subjected to 10 μM cytochalasin B was reduced by approximately 25% compared to cells not treated with the inhibitor. Importantly, these results agreed with the fluorescence observed from cell lysates when a commercially available glucose assay kit was used.

### Results

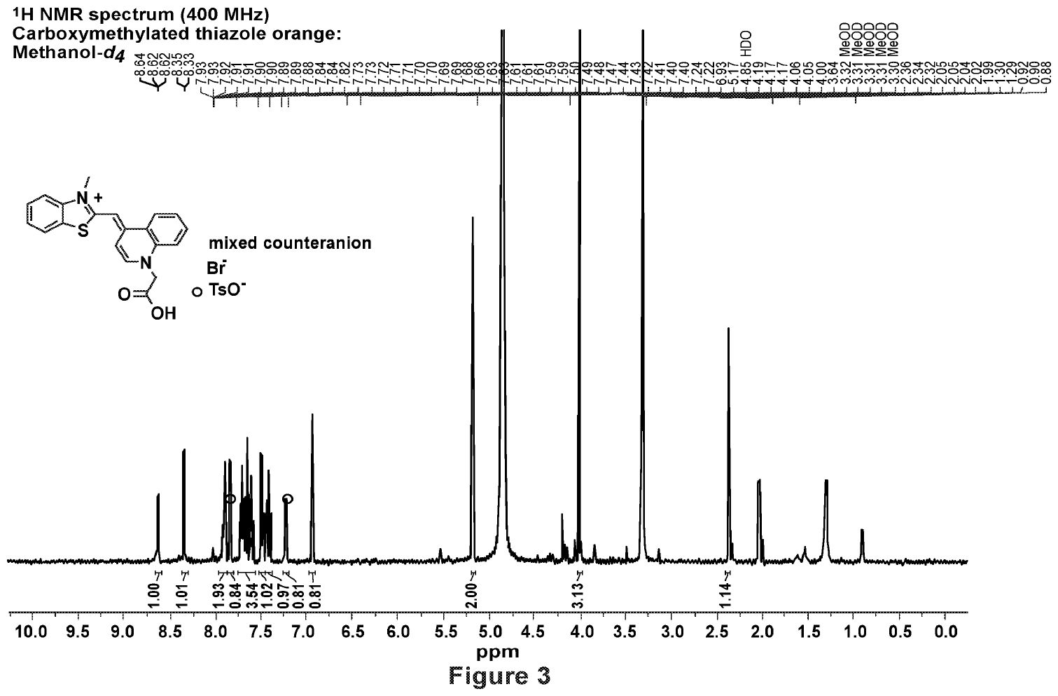

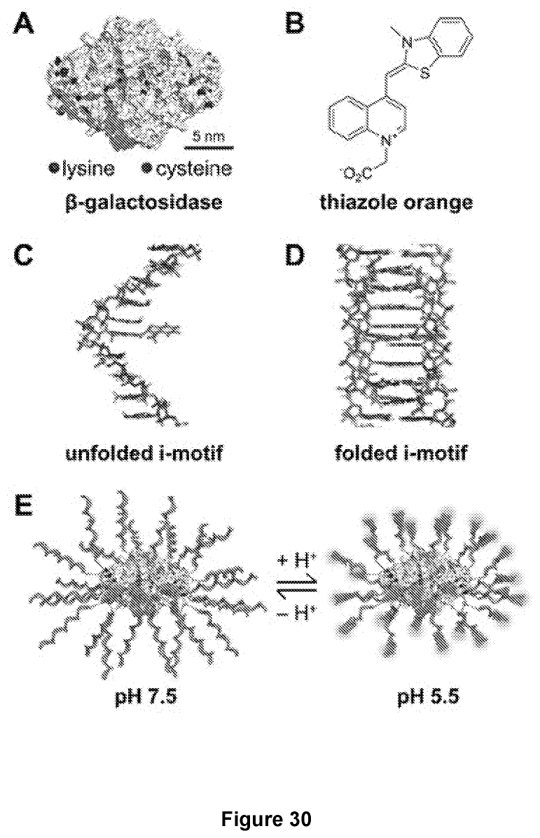

In various aspects, the present disclosure provides a quencher-free strategy coupled to an SNA architecture. To demonstrate, SNAs in which the nucleic acid sequences act as the recognition element were designed. A duplex-sensitive dye, thiazole orange (TO), was used as a base-surrogate in an oligonucleotide recognition sequence that is designed to bind to the target analyte (FIGS. 3 and 30). This class of dyes, derived from intercalators, have low fluorescence in a single-stranded oligonucleotide due to unrestricted rotation about the methine bridge in the molecules.³² In contrast, upon binding to the target analyte, the dye undergoes forced intercalation (FIT) between the oligonucleotide base pairs, thereby restricting its motion, leading to enhanced fluorescence.³²⁻³⁴ The use of a duplex-sensitive dye means that any nanoparticle core may be utilized. Here, a protein core was used as a model system due to its well-established biocompatibility, biodegradability, and known chemical topology, which allowed for site-specific oligonucleotide attachment.^{35,36}

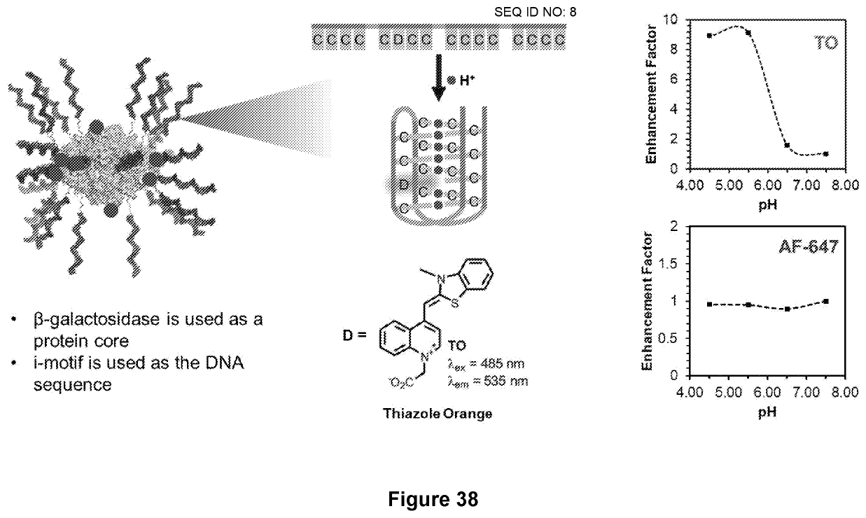

For a proof-of-concept, an i-motif sequence was chosen as the recognition strand that undergoes pH-dependent structural changes between an unfolded and a tetraplex form.^{37,38} The i-motif is an aptamer for protons, and the i-motif was converted into a FIT-aptamer using a strategy previously reported.³⁴ β-galactosidase (β-gal) was chosen as the protein core as it has been used in previous ProSNA studies.^{14,15} A fluorescent dye, Alexa Fluor 647 (AF-647), was covalently conjugated to the cysteine residues of the protein through maleimide-thiol coupling to enable the monitoring of the cellular uptake of the probe. Dibenzocyclooctyne terminated DNA was attached orthogonally to the lysine residues modified with PEG-azides (FIG. 36). These modifications gave rise to a TO containing i-motif-β-gal ProSNA termed ProTOn (FIGS. 30 and 38).

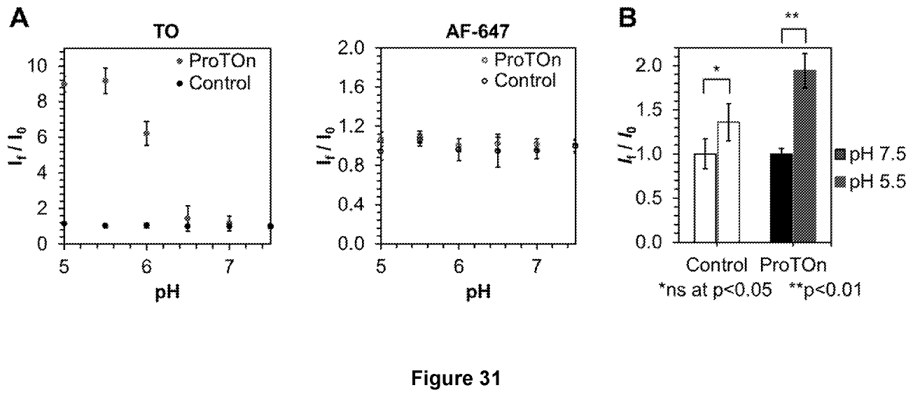

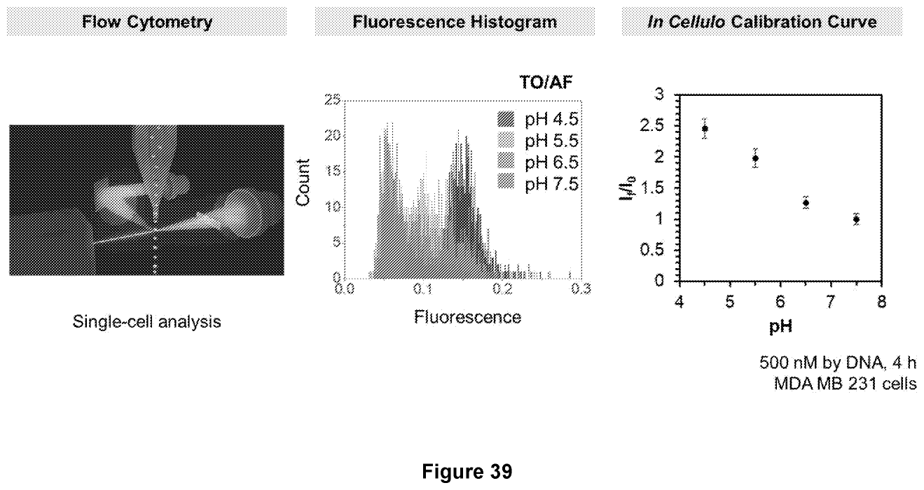

It was first established that ProTOn was capable of detecting pH changes in vitro (FIGS. 31A, 38, and 39) by adding it to buffered solutions at different pH. The results showed a gradual increase in the fluorescence enhancement of TO, saturating at 9-fold, when the pH of the solution changes from 7.5 to 5.0. No change in fluorescence signal was observed from AF-647 in this pH range. Similarly, ProSNAs containing a control sequence that does not form the i-motif structure resulted in no fluorescence enhancement either in the TO channel or the AF-647 channel in this pH range.

Next, the ability of ProTOn to report changes in intracellular pH was determined (FIGS. 31B, 38, and 39). The MDA-MB-231 cell line, a human epithelial breast cancer cell line, was chosen as a model. These cells were incubated with 500 nM ProTOn (by DNA) in serum-free media for 3 h. The cells were then clamped at pH 5.5 and pH 7.5 and analyzed by flow cytometry. The mean fluorescence of cells at pH 5.5 was almost double that of cells at pH 7.5, consistent with the results obtained when pHrodo™ Red AM, a commercially available intracellular pH probe, was used as a benchmark (FIG. 10). As before, control ProSNAs did not elicit a significant fluorescence change, demonstrating the specificity of ProTOn.

Importantly, this new quencher-free ProSNA-based design was not prone to false-positive signals, as evidenced by in vitro nuclease degradation experiments (FIGS. 6 and 8) and pulse-chase experiments in MDA-MB-231 cells (FIGS. 7 and 9). Moreover, the use of a FIT-strategy does not require strand displacement, overcoming limitations associated with non-specific dehybridization of the reporter strand and allowing faster binding to the target analyte.³⁴ Taken together, these results showed that ProSNA-based quencher-free probes constitute a next-generation platform for monitoring intracellular analytes in live cells.

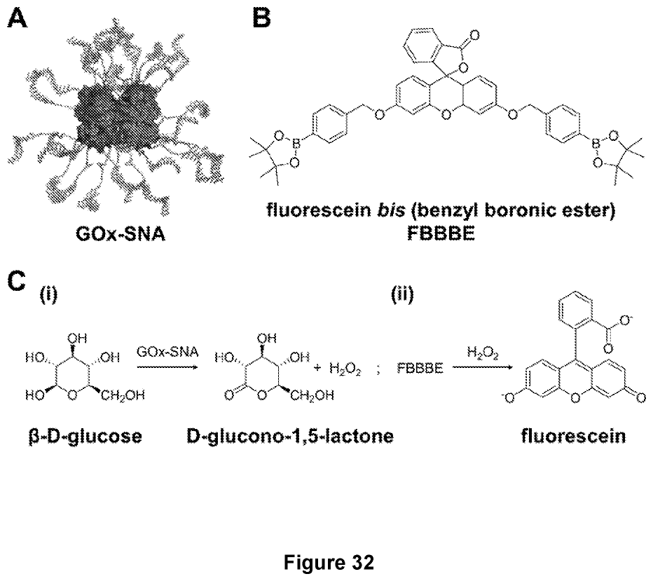

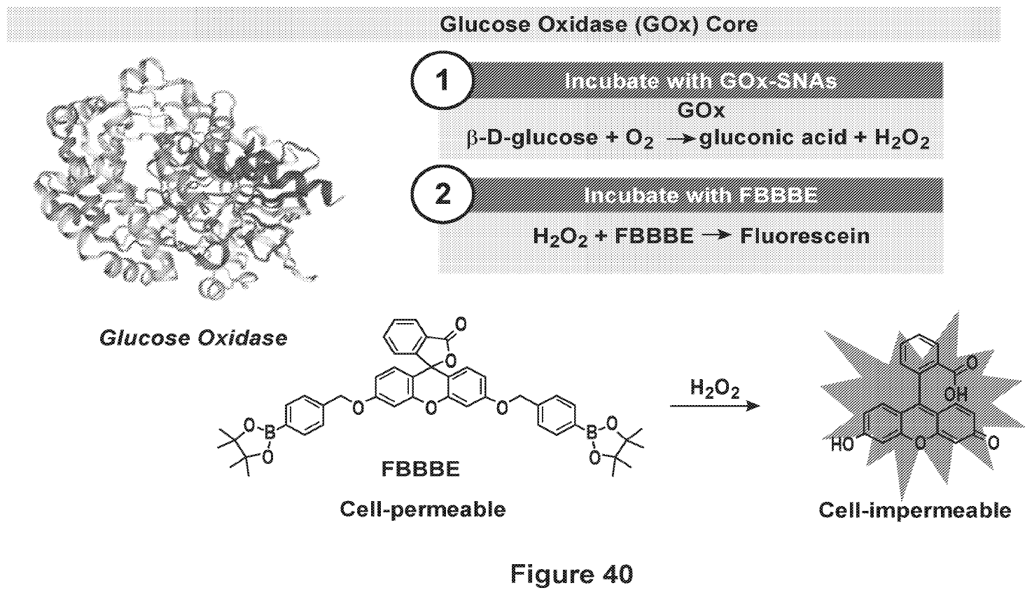

Importantly, the use of a ProSNA allows one to not only detect analytes through the nucleic acid shell but also vastly expands the range of analytes that can be detected by taking advantage of the functional protein core. It was hypothesized that using an enzyme, analytes for which nucleic acid-based recognition sequences are not known can be detected. To test this hypothesis, a ProSNA for intracellular glucose detection was designed using glucose oxidase (GOx) as the core (FIGS. 32 and 40). Glucose was chosen as a model analyte because of its fundamental importance to maintaining cellular functions, its high intracellular abundance (approximately 0.1 mM-2 mM),^{39,40} and the lack of a glucose aptamer with a biologically relevant binding affinity.⁴¹ Remarkably, the activity of GOx-SNAs remained unchanged compared to the native protein (FIG. 13).

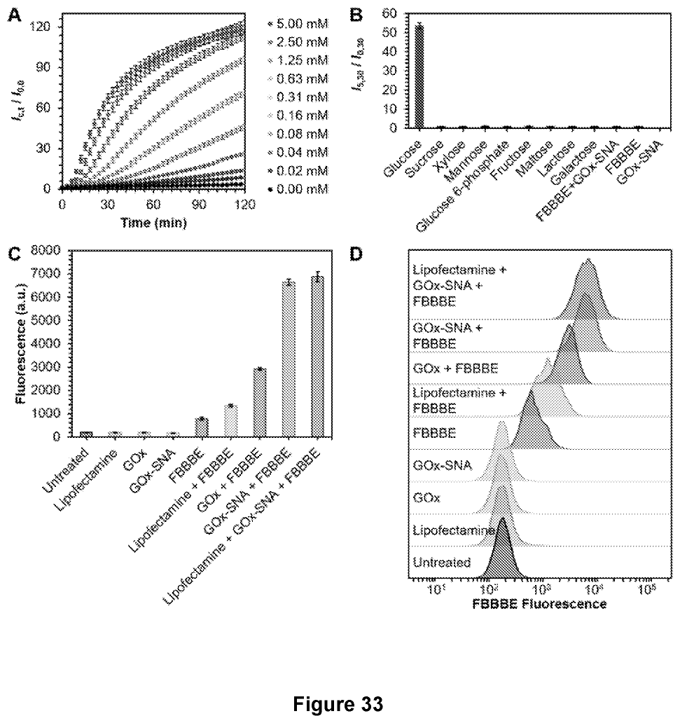

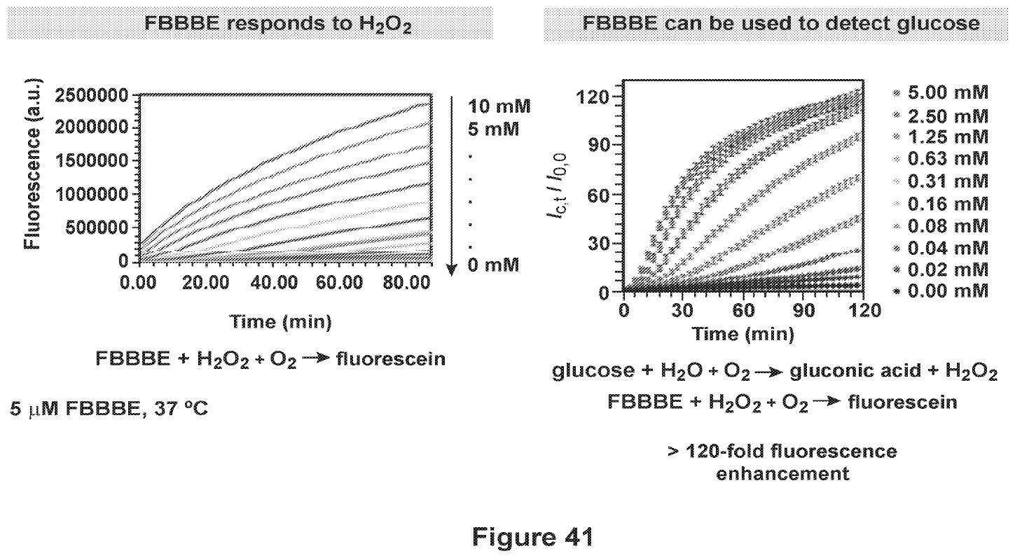

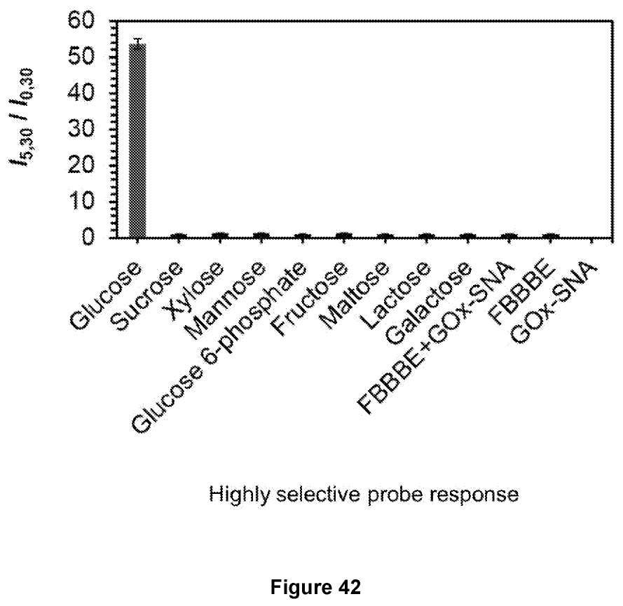

To detect glucose intracellularly, a two-step assay was developed. In the first step, cells are treated with GOx-SNAs in glucose-free media. Upon entering the cells, GOx-SNAs catalyze the conversion of glucose to D-glucono-1,5-lactone and produce hydrogen peroxide. In the second step, the cells are washed thoroughly and treated with fluorescein bis (benzyl boronic ester), FBBBE, a cell-permeable, non-fluorescent fluorescein derivative.⁴² In the presence of hydrogen peroxide, the boronate groups are cleaved and highly fluorescent fluorescein is formed and retained intracellularly. Therefore, the fluorescence is directly proportional to the amount of glucose in the cell. This assay resulted in a 120-fold fluorescence enhancement in the presence of glucose in vitro (FIGS. 33A and 41). Due to the high specificity of GOx, the probe is selective against other sugars including sucrose, xylose, mannose, fructose, maltose, lactose, galactose, as well as glucose-6-phosphate which results from rapid phosphorylation of glucose upon cellular entry (FIG. 33B, FIG. 16), and FIG. 42).⁴³

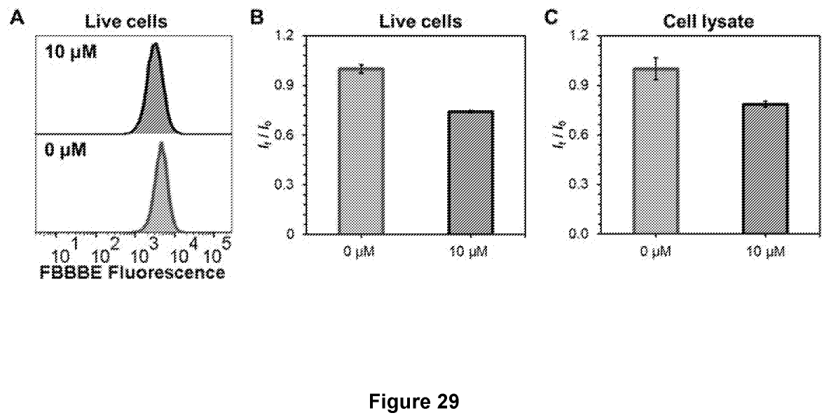

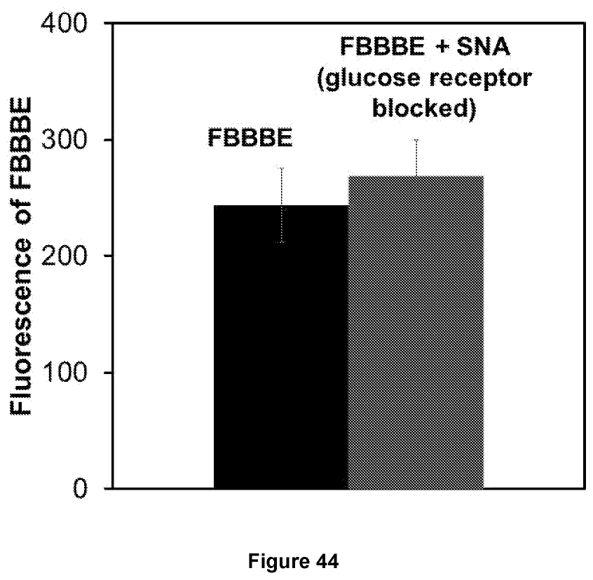

To ensure the validity of this new assay in cells, nine different cells lines (MDA-MB-231, MC38, U87, SKOV3, HDF, EL4, EG7-OVA, 4T1, and NIH/3T3) were tested, representing a mix of cancer and normal cells, adherent and suspension cells, and human and murine-derived cells (FIGS. 18-26 and 43). Flow cytometry experiments show that cells treated with FBBBE alone have increased fluorescence compared to untreated cells. These results were expected because some fluorescein is formed due to the basal level of H₂O₂ produced in the cells. Cells pre-treated with 40 nM GOx-SNAs (by protein) showed up to an approximate 12-fold increase in fluorescence due to the elevated levels of H₂O₂ produced (FIG. 33C). Modulating intracellular glucose levels impacted the fluorescence detected. GOx-SNA-treated cells exposed to media containing 25 mM glucose showed an increase in fluorescence compared to those exposed to 0 mM glucose (FIG. 27). Similarly, increasing glucose uptake by treating cells with 100 nM insulin resulted in higher fluorescence (FIG. 28). In contrast, decreasing glucose uptake by using 10 μM cytochalasin B, a well-known glucose transport inhibitor,⁴⁴ resulted in decreased fluorescence (FIGS. 29 and 44). Taken together, these experiments showed that GOx-SNAs can be used as intracellular glucose probes, which going forward, may aid in the high throughput screening of antihyperglycemic drugs.

### Conclusions

In conclusion, the foregoing experiments demonstrated the effectiveness of intracellular probes based on ProSNAs. Their modular structure allows one to change the protein core and the nucleic acid shell independently and detect analytes using either component as the sensing moiety through binding- or activity-based sensing. The programmable nature of nucleic acids allows for the detection of targets using aptamers, DNAzymes, or hybridization-based probes. The unparalleled specificity of proteins will give rise to probes that are highly selective for their targets. When one takes into account the many proteins that have been developed for sensing analytes in cell lysates or fluorescent proteins that are genetically encoded, the scope of possibilities is enormous, considering that these proteins could be repurposed as exogenous probes for detecting intracellular analytes in live cells and clinical samples.

### Example 2

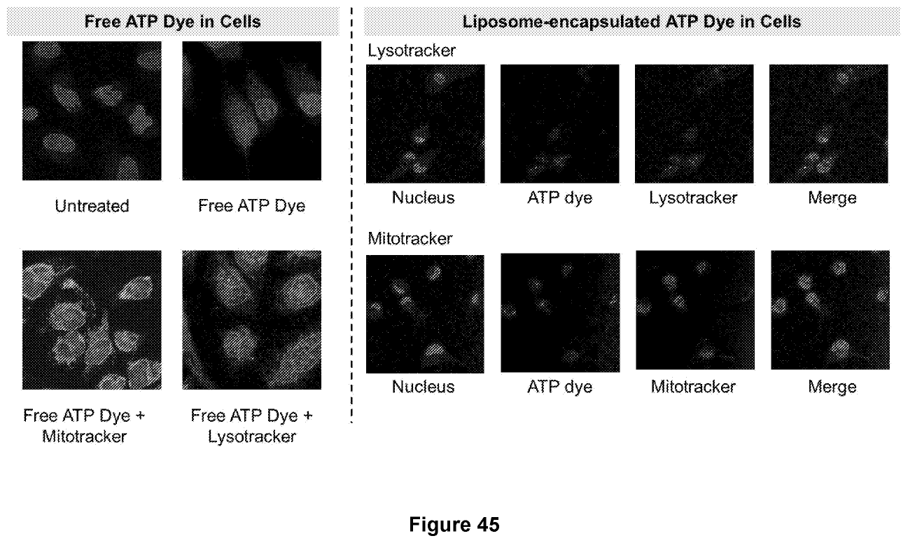

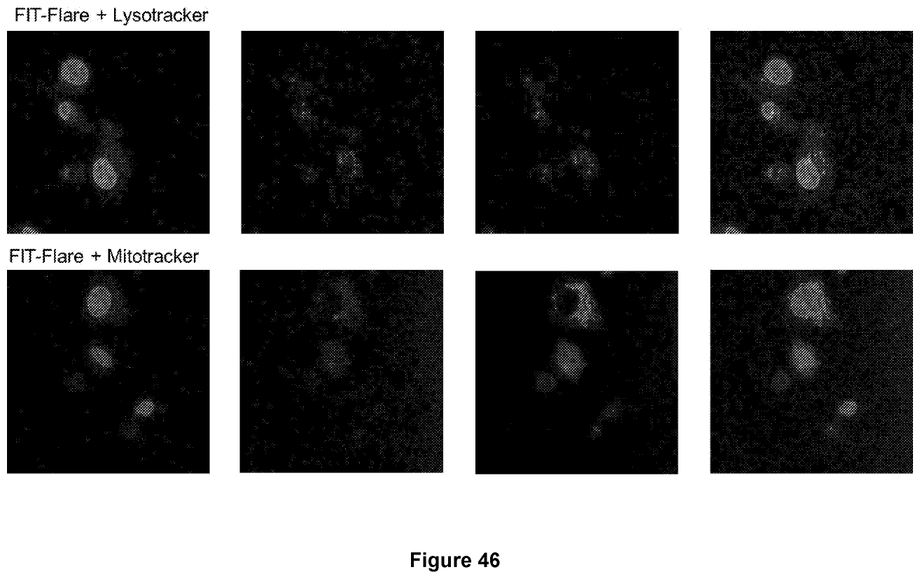

In this example, it is demonstrated that the uptake pathway of an ATP-sensitive dye (ATP Red) can be changed by encapsulating it in liposomal SNAs. In the design used, the DNA shell does not detect analytes and is only used to facilitate cellular uptake. Encapsulated ATP Red entered through the endolysosomal pathway and enabled the detection of ATP along this pathway, whereas the free dye localizes to the mitochondria and detects mitochondrial ATP. The localization of the ATP Red dye is tracked using Lysotracker and Mitotracker which light up the endolysosomes and mitochondria, respectively. Colocalization of fluorescence signals from Lysotracker/Mitotracker with the ATP Red signal indicates where in the cell ATP is being detected. See FIGS. 45 and 46.

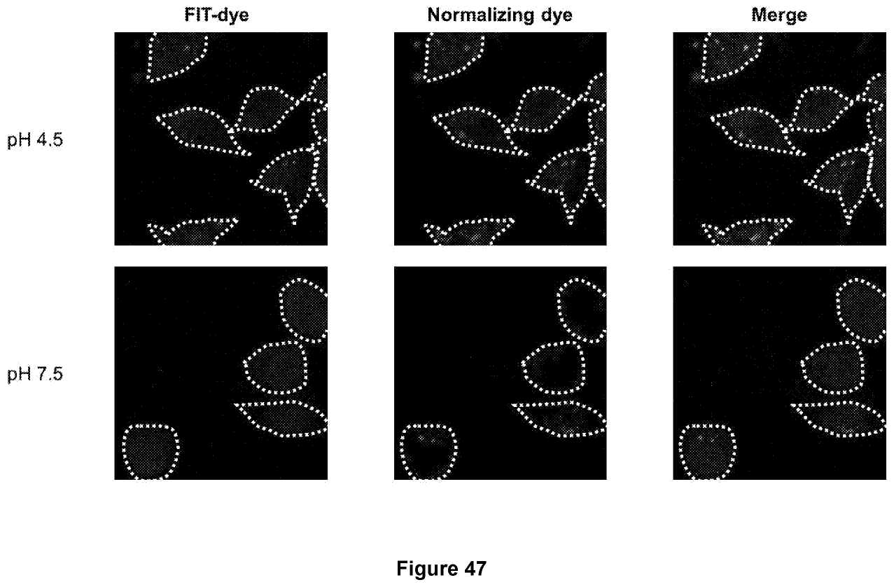

FIG. 47 shows confocal images of cells clamped at pH 4.5 and pH 7.5 after being treated with ProTOn. The signal from the normalizing dye channel was used to account for probe uptake whereas the signal from the FIT-dye channel indicated the pH.

### REFERENCES

(1) Dean, K. M.; Palmer, A. E. Advances in Fluorescence Labeling Strategies for Dynamic Cellular Imaging. Nat. Chem. Biol. 2014, 10 (7), 512-523.
(2) Leung, K.; Chakraborty, K.; Saminathan, A.; Krishnan, Y. A DNA Nanomachine Chemically Resolves Lysosomes in Live Cells. Nat. Nanotechnol. 2019, 14 (2), 176-183.
(3) Samanta, D.; Ebrahimi, S. B.; Mirkin, C. A. Nucleic-Acid Structures as Intracellular Probes for Live Cells. Adv. Mater. 2019, 32 (13), 1901743.
(4) Valm, A. M.; Cohen, S.; Legant, W. R.; Melunis, J.; Hershberg, U.; Wait, E.; Cohen, A. R.; Davidson, M. W.; Betzig, E.; Lippincott-Schwartz, J. Applying Systems-Level Spectral Imaging and Analysis to Reveal the Organelle Interactome. Nature 2017, 546 (7656), 162-167.
(5) Park, S.; Aalipour, A.; Vermesh, O.; Yu, J. H.; Gambhir, S. S. Towards Clinically Translatable in Vivo Nanodiagnostics. Nat. Rev. Mater. 2017, 2 (5), 17014.
(6) Ebrahimi, S. B.; Samanta, D.; Mirkin, C. A. DNA-Based Nanostructures for Live-Cell Analysis. J. Am. Chem. Soc. 2020, 142 (26), 11343-11356.
(7) Schaferling, M. The Art of Fluorescence Imaging with Chemical Sensors. Angew. Chemie Int. Ed. 2012, 51 (15), 3532-3554.
(8) Bruemmer, K. J.; Crossley, S. W. M.; Chang, C. J. Activity-Based Sensing: A Synthetic Methods Approach for Selective Molecular Imaging and Beyond. Angew. Chemie Int. Ed. 2020, doi.org/10.1002/anie.201909690.
(9) Konstantinou, G. N. Enzyme-Linked Immunosorbent Assay (ELISA). In Methods in Molecular Biology; 2017; pp 79-94.
(10) Greenwald, E. C.; Mehta, S.; Zhang, J. Genetically Encoded Fluorescent Biosensors Illuminate the Spatiotemporal Regulation of Signaling Networks. Chem. Rev. 2018, 118 (24), 11707-11794.
(11) Paige, J. S.; Nguyen-Duc, T.; Song, W.; Jaffrey, S. R. Fluorescence Imaging of Cellular Metabolites with RNA. Science 2012, 335 (6073), 1194.
(12) Nolan, T.; Hands, R. E.; Bustin, S. A. Quantification of MRNA Using Real-Time RT-PCR. Nat. Protoc. 2006, 1 (3), 1559-1582.
(13) Trask, B. J. Human Cytogenetics: 46 Chromosomes, 46 Years and Counting. Nat. Rev. Genet. 2002, 3 (10), 769-778.
(14) Brodin, J. D.; Sprangers, A. J.; McMillan, J. R.; Mirkin, C. A. DNA-Mediated Cellular Delivery of Functional Enzymes. J. Am. Chem. Soc. 2015, 137 (47), 14838-14841.
(15) Kusmierz, C. D.; Bujold, K. E.; Callmann, C. E.; Mirkin, C. A. Defining the Design Parameters for in Vivo Enzyme Delivery Through Protein Spherical Nucleic Acids. ACS Cent. Sci. 2020, 6 (5), 815-822.
(16) Cutler, J. I.; Auyeung, E.; Mirkin, C. A. Spherical Nucleic Acids. J. Am. Chem. Soc. 2012, 134 (3), 1376-1391.
(17) Seferos, D. S.; Giljohann, D. A.; Hill, H. D.; Prigodich, A. E.; Mirkin, C. A. Nano-Flares: Probes for Transfection and MRNA Detection in Living Cells. J. Am. Chem. Soc. 2007, 129 (50), 15477-15479.
(18) Chinen, A. B.; Guan, C. M.; Ferrer, J. R.; Barnaby, S. N.; Merkel, T. J.; Mirkin, C. A. Nanoparticle Probes for the Detection of Cancer Biomarkers, Cells, and Tissues by Fluorescence. Chem. Rev. 2015, 115 (19), 10530-10574.
(19) Chen, T.; Wu, C. S.; Jimenez, E.; Zhu, Z.; Dajac, J. G.; You, M.; Han, D.; Zhang, X.; Tan, W. DNA Micelle Flares for Intracellular MRNA Imaging and Gene Therapy. Angew. Chemie—Int. Ed. 2013, 52 (7), 2012-2016.
(20) Vilela, P.; Heuer-Jungemann, A.; EI-Sagheer, A.; Brown, T.; Muskens, O. L.; Smyth, N. R.; Kanaras, A. G. Sensing of Vimentin MRNA in 2D and 3D Models of Wounded Skin Using DNA-Coated Gold Nanoparticles. Small 2018, 14 (12), 1703489.
(21) Jayagopal, A.; Halfpenny, K. C.; Perez, J. W.; Wright, D. W. Hairpin DNA-Functionalized Gold Colloids for the Imaging of MRNA in Live Cells. J. Am. Chem. Soc. 2010, 132 (28), 9789-9796.
(22) Li, N.; Chang, C.; Pan, W.; Tang, B. A Multicolor Nanoprobe for Detection and Imaging of Tumor-Related Mrnas in Living Cells. Angew. Chemie—Int. Ed. 2012, 51 (30), 7426-7430.
(23) Zheng, D.; Seferos, D. S.; Giljohann, D. A.; Patel, P. C.; Mirkin, C. A. Aptamer Nano-Flares for Molecular Detection in Living Cells. Nano Lett. 2009, 9 (9), 3258-3261.
(24) Wu, P.; Hwang, K.; Lan, T.; Lu, Y. A DNAzyme-Gold Nanoparticle Probe for Uranyl Ion in Living Cells. J. Am. Chem. Soc. 2013, 135 (14), 5254-5257.
(25) Chen, T.-T.; Tian, X.; Liu, C.-L.; Ge, J.; Chu, X.; Li, Y. Fluorescence Activation Imaging of Cytochrome c Released from Mitochondria Using Aptameric Nanosensor. J. Am. Chem. Soc. 2015, 137 (2), 982-989.
(26) Halo, T. L.; McMahon, K. M.; Angeloni, N. L.; Xu, Y.; Wang, W.; Chinen, A. B.; Malin, D.; Strekalova, E.; Cryns, V. L.; Cheng, C.; Mirkin, C. A.; Thaxton, C. S. NanoFlares for the Detection, Isolation, and Culture of Live Tumor Cells from Human Blood. Proc. Natl. Acad. Sci. U.S.A 2014, 111 (48), 17104-17109.
(27) Yeo, D. C.; Wiraja, C.; Paller, A. S.; Mirkin, C. A.; Xu, C. Abnormal Scar Identification with Spherical-Nucleic-Acid Technology. Nat. Biomed. Eng. 2018, 2, 227-238.
(28) Liu, Z.; Zhao, J.; Zhang, R.; Han, G.; Zhang, C.; Liu, B.; Zhang, Z.; Han, M. Y.; Gao, X. Cross-Platform Cancer Cell Identification Using Telomerase-Specific Spherical Nucleic Acids. ACS Nano 2018, 12 (4), 3629-3637.
(29) Conde, J.; Oliva, N.; Artzi, N. Implantable Hydrogel Embedded Dark-Gold Nanoswitch as a Theranostic Probe to Sense and Overcome Cancer Multidrug Resistance. Proc. Natl. Acad. Sci. U.S.A 2015, 112 (11), E1278-E1287.
(30) Yang, Y.; Huang, J.; Yang, X.; Quan, K.; Wang, H.; Ying, L.; Xie, N.; Ou, M.; Wang, K. FRET Nanoflares for Intracellular MRNA Detection: Avoiding False Positive Signals and Minimizing Effects of System Fluctuations. J. Am. Chem. Soc. 2015, 137 (26), 8340-8343.
(31) Wilson, B. D.; Hariri, A. A.; Thompson, I. A. P.; Eisenstein, M.; Soh, H. T. Independent Control of the Thermodynamic and Kinetic Properties of Aptamer Switches. Nat. Commun. 2019, 10 (1), 5079.
(32) Kohler, O.; Jarikote, D. V.; Seitz, O. Forced Intercalation Probes (FIT Probes): Thiazole Orange as a Fluorescent Base in Peptide Nucleic Acids for Homogeneous Single-Nucleotide-Polymorphism Detection. ChemBioChem 2005, 6 (1), 69-77.
(33) Hövelmann, F.; Gaspar, I.; Chamiolo, J.; Kasper, M.; Steffen, J.; Ephrussi, A.; Seitz, O. LNA-Enhanced DNA FIT-Probes for Multicolour RNA Imaging. Chem. Sci. 2016, 7 (1), 128-135.
(34) Ebrahimi, S. B.; Samanta, D.; Cheng, H. F.; Nathan, L. I.; Mirkin, C. A. Forced Intercalation (FIT)-Aptamers. J. Am. Chem. Soc. 2019, 141 (35), 13744-13748.
(35) Abascal, N. C.; Regan, L. The Past, Present and Future of Protein-Based Materials. Open Biol. 2018, 8 (10), 180113.
(36) Krall, N.; da Cruz, F. P.; Boutureira, O.; Bernardes, G. J. L. Site-Selective Protein-Modification Chemistry for Basic Biology and Drug Development. Nat. Chem. 2016, 8 (2), 103-113.
(37) Chakraborty, K.; Veetil, A.; Jaffrey, S. R.; Krishnan, Y. Nucleic Acid-Based Nanodevices in Biological Imaging. Annu. Rev. Biochem. 2016, 85, 349-373.
(38) Choi, J.; Kim, S.; Tachikawa, T.; Fujitsuka, M.; Majima, T. PH-Induced Intramolecular Folding Dynamics of i-Motif DNA. J. Am. Chem. Soc. 2011, 133 (40), 16146-16153.
(39) Cline, G. W.; Petersen, K. F.; Krssak, M.; Shen, J.; Hundal, R. S.; Trajanoski, Z.; Inzucchi, S.; Dresner, A.; Rothman, D. L.; Shulman, G. I. Impaired Glucose Transport as a Cause of Decreased Insulin-Stimulated Muscle Glycogen Synthesis in Type 2 Diabetes. N. Engl. J. Med. 1999, 341 (4), 240-246.
(40) Nascimento, R. A. S.; Ozel, R. E.; Mak, W. H.; Mulato, M.; Singaram, B.; Pourmand, N. Single Cell “Glucose Nanosensor” Verifies Elevated Glucose Levels in Individual Cancer Cells. Nano Lett. 2016, 16 (2), 1194-1200.
(41) Nakatsuka, N.; Yang, K.-A.; Abendroth, J. M.; Cheung, K. M.; Xu, X.; Yang, H.; Zhao, C.; Zhu, B.; Rim, Y. S.; Yang, Y.; Weiss, P. S.; Stojanovic, M. N.; Andrews, A. M. Aptamer-Field-Effect Transistors Overcome Debye Length Limitations for Small-Molecule Sensing. Science 2018, 362 (6412), 319-324.
(42) Daniel, K. B.; Agrawal, A.; Manchester, M.; Cohen, S. M. Readily Accessible Fluorescent Probes for Sensitive Biological Imaging of Hydrogen Peroxide. ChemBioChem 2013, 14 (5), 593-598.
(43) Hers, H. G.; Hue, L. Gluconeogenesis and Related Aspects of Glycolysis. Annu. Rev. Biochem. 1983, 52 (1), 617-653.
(44) Estensen, R. D.; Plagemann, P. G. W. Cytochalasin B: Inhibition of Glucose and Glucosamine Transport. Proc. Natl. Acad. Sci. 1972, 69 (6), 1430-1434.

What is claimed is:

1. A method for detecting a target analyte comprising the step of contacting the target analyte with a spherical nucleic acid (SNA), the SNA comprising a nanoparticle core and an oligonucleotide attached thereto, wherein the oligonucleotide comprises a detectable marker situated at an internal location within the oligonucleotide, wherein the contacting results in binding of the target analyte to the oligonucleotide, and wherein the binding results in restriction of internal rotation of the marker, resulting in a detectable change and thereby detecting the target analyte.

2. The method of claim 1, wherein a plurality of the oligonucleotides is attached thereto.

3. The method of claim 1 or claim 2, wherein the detectable change is an increase in fluorescence.

4. The method of any one of claims 1-3, wherein the oligonucleotide is an aptamer.

5. The method of any one of claims 1-4, wherein target analyte binding to the oligonucleotide results in forced intercalation (FIT) of the marker between base pairs of the oligonucleotide.

6. The method of any one of claims 1-5, wherein the detectable marker is a marker with internal rotation-dependent fluorescence.

7. The method of claim 6, wherein the detectable marker is a viscosity-sensitive marker.

8. The method of any one of claims 1-7, wherein the detectable marker is thiazole orange (TO), quinoline blue, quinoline violet, thiazole red, a derivative thereof, or a cyanine derivative.

9. The method of any one of claims 1-8, wherein the detectable change is proportional to concentration of the target analyte.

10. The method of any one of claims 1-9, wherein the target analyte is a protein, an ion, a small molecule, a lipid, a carbohydrate, an oligosaccharide, a cell, a nucleic acid, or a combination thereof.

11. The method of claim 10, wherein the ion is a metal ion.

12. The method of claim 11, wherein the metal ion is a mercury ion, a copper ion, a silver ion, a zinc ion, a gold ion, a manganese ion, or a combination thereof.

13. The method of claim 10, wherein the ion is a hydrogen ion.

14. The method of claim 13, wherein the detectable change is indicative of a pH change.

15. The method of any one of claims 1-14, wherein the oligonucleotide is DNA, RNA, or a modified form thereof.

16. The method of any one of claims 1-15, wherein the oligonucleotide is about 5 to about 1000 nucleotides in length.

17. The method of any one of claims 1-16, wherein the oligonucleotide is about 10 to about 100 nucleotides in length.

18. The method of any one of claims 1-17, wherein the oligonucleotide comprises a spacer.

19. The method of any one of claims 1-18, wherein the detectable marker is situated at a position that is x nucleotides from a terminus of the oligonucleotide, wherein x is an integer that is 1, n/2, or any integer between 1 and n/2, wherein n is (i) the length of the oligonucleotide and (ii) an even number.

20. The method of any one of claims 1-18, wherein the detectable marker is situated at a position that is x nucleotides from a terminus of the oligonucleotide, wherein x is an integer that is 1, (n+1)/2, or any integer between 1 and (n+1)/2, wherein n is (i) the length of the oligonucleotide and (ii) an odd number.

21. The method of any one of claims 1-20, wherein the detectable marker is situated at a position that is 1, 2, 3, 4, 5, 6, 7, 8, 9, or 10 nucleotides from a terminus of the oligonucleotide.

22. The method of any one of claims 1-21, wherein the SNA further comprises an inhibitory oligonucleotide attached thereto.

23. The method of claim 22, wherein the inhibitory oligonucleotide is antisense DNA, small interfering RNA (siRNA), an aptamer, a short hairpin RNA (shRNA), a DNAzyme, or an aptazyme.

24. The method of any one of claims 1-23, wherein the SNA further comprises an immunostimulatory oligonucleotide attached thereto.

25. The method of claim 24, wherein the immunostimulatory oligonucleotide is a toll-like receptor (TLR) agonist.

26. The method of claim 25, wherein the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof.

27. The method of any one of claims 1-26, wherein the SNA further comprises a toll-like receptor (TLR) antagonist.

28. The method of claim 27, wherein the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof.

29. A method for identifying a nucleotide recognition sequence that is useful to detect a target analyte comprising the steps of:
contacting the target analyte with a spherical nucleic acid (SNA), the SNA comprising a nanoparticle core and an aptamer attached thereto, wherein the aptamer comprises a candidate nucleotide sequence and a detectable marker situated at an internal location within the aptamer, wherein binding of the candidate nucleotide sequence to the target analyte results in an increase in fluorescence due to restriction of internal rotation of the detectable marker;
comparing fluorescence before and after the contacting, and
identifying the candidate nucleotide sequence as the nucleotide recognition sequence from an increase in fluorescence after the contacting.

30. The method of claim 29, wherein target analyte binding to the aptamer results in forced intercalation (FIT) of the marker between oligonucleotide base pairs of the aptamer.

31. The method of claim 29 or claim 30, wherein a plurality of the aptamers are attached thereto.

32. A method for detecting a target analyte comprising the step of contacting the target analyte with a spherical nucleic acid (SNA), the SNA comprising a nanoparticle core and a plurality of oligonucleotides attached thereto, the plurality of oligonucleotides comprising:
(a) an aptamer or portion thereof comprising (i) nucleotide sequence X, (ii) nucleotide sequence Y which binds to the target analyte, either alone or in combination with nucleotide sequence Y′, and (iii) a detectable marker situated at an internal location within the aptamer, and
(b) an additional aptamer or portion thereof comprising (i) nucleotide sequence X′ which is sufficiently complementary to hybridize to nucleotide sequence X, and (ii) nucleotide sequence Y′ which binds to the target analyte, either alone or in combination with nucleotide sequence Y,
wherein the contacting results in hybridization of nucleotide sequence X with nucleotide sequence X′ and binding of the target analyte with nucleotide sequence Y and nucleotide sequence Y′, wherein
the binding of nucleotide sequence X with nucleotide sequence X′ and the target analyte with nucleotide sequence Y and nucleotide sequence Y′ result in restriction of internal rotation of the marker, resulting in a detectable change and thereby detecting the target analyte.

33. The method of claim 32, wherein nucleotide sequence Y and nucleotide sequence Y′ bind to different binding sites of the target analyte.

34. The method of claim 32, wherein nucleotide sequence Y and nucleotide sequence Y′ together bind to the same binding site of the target analyte.

35. The method of any one of claims 32-34, wherein the binding of nucleotide sequence X with nucleotide sequence X′ and the target analyte with nucleotide sequence Y and nucleotide sequence Y′ result in forced intercalation (FIT) of the marker between oligonucleotide base pairs of the aptamer and the additional aptamer.

36. The method of any one of claims 32-35, wherein the detectable marker is situated at a position that is x nucleotides from a terminus of the aptamer, wherein x is an integer that is 1, n/2, or any integer between 1 and n/2, wherein n is (i) the length of the aptamer and (ii) an even number.

37. The method of any one of claims 32-35, wherein the detectable marker is situated at a position that is x nucleotides from a terminus of the aptamer, wherein x is an integer that is 1, (n+1)/2, or any integer between 1 and (n+1)/2, wherein n is (i) the length of the aptamer and (ii) an odd number.

38. The method of any one of claims 32-37, wherein the detectable marker is situated at a position that is 1, 2, 3, 4, 5, 6, 7, 8, 9, or 10 nucleotides from a terminus of the aptamer.

39. The method of any one of claims 32-38, wherein the plurality of oligonucleotides comprises an inhibitory oligonucleotide.

40. The method of claim 39, wherein the inhibitory oligonucleotide is antisense DNA, small interfering RNA (siRNA), an aptamer, a short hairpin RNA (shRNA), a DNAzyme, or an aptazyme.

41. The method of any one of claims 32-40, wherein the plurality of oligonucleotides comprises an immunostimulatory oligonucleotide.

42. The method of claim 41, wherein the immunostimulatory oligonucleotide is a toll-like receptor (TLR) agonist.

43. The method of claim 42, wherein the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof.

44. The method of any one of claims 32-43, wherein the plurality of oligonucleotides comprises a toll-like receptor (TLR) antagonist.

45. The method of claim 44, wherein the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof.

46. The method of any one of claims 1-45, wherein the nanoparticle core is a metallic core, a semiconductor core, an insulator core, an upconverting core, a micellar core, a dendrimer core, a liposomal core, a polymer core, a metal-organic framework core, a protein core, or a combination thereof.

47. The method of claim 46, wherein the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), or chitosan.

48. The method of claim 46, wherein the polymer is poly(lactic-co-glycolic acid) (PLGA).

49. The method of claim 46, wherein the nanoparticle core is gold, silver, platinum, aluminum, palladium, copper, cobalt, indium, cadmium selenide, iron oxide, fullerene, metal-organic framework, zinc sulfide, or nickel.

50. The method of claim 46, wherein the nanoparticle core is a protein core.

51. The method of claim 46 or claim 50, wherein contacting the protein core with the target analyte results in an additional detectable change.

52. The method of claim 51, wherein the additional detectable change is a fluorescence change or a luminescence change.

53. The method of claim 51, wherein the protein core is an enzyme, a therapeutic protein, a structural protein, a defensive protein, a storage protein, a transport protein, a hormone, a receptor protein, a motor protein, or a fluorescent protein.

54. The method of claim 50, wherein the protein core comprises an enzyme that interacts with and allows detection of an additional target analyte.

55. The method of claim 54, wherein the target analyte and the additional target analyte are the same.

56. The method of claim 53 or claim 54, wherein the enzyme is glucose oxidase (GOx), cholesterol oxidase, luciferase, or creatinine deaminase.

57. The method of any one of claims 54-56, further comprising contacting the additional target analyte with an agent.

58. The method of claim 57, wherein the agent is associated with the external side of the nanoparticle core.

59. The method of claim 57 or claim 58, wherein the agent is encapsulated in the nanoparticle core.

60. The method of any one of claims 57-59, wherein the agent is associated with the oligonucleotide.

61. The method of any one of claims 57-60, wherein the agent is added exogenously.

62. The method of any one of claims 54-61, wherein the additional target analyte is detectable after contacting the additional target analyte with the agent.

63. The method of any one of claims 57-62, wherein the agent is a small molecule.

64. The method of claim 63, wherein the small molecule is a dye or a luminophore.

65. The method of claim 64, wherein the dye is a normalizing dye, or a dye that localizes to an organelle.

66. A method for detecting a target analyte comprising the step of:
contacting the target analyte with a spherical nucleic acid (SNA) and an agent, the SNA comprising a protein core and an oligonucleotide attached thereto, wherein the contacting of the protein core with the target analyte results in a change in the target analyte that is detectable by the agent, thereby detecting the target analyte.

67. The method of claim 66, wherein the SNA comprises a plurality of oligonucleotides attached thereto.

68. The method of claim 66, wherein the change that is detectable by the agent is a fluorescence change or a luminescence change.

69. The method of claim 66, wherein the protein core comprises an enzyme.

70. The method of claim 69, wherein the enzyme is glucose oxidase (GOx), cholesterol oxidase, luciferase, or creatinine deaminase.

71. The method of any one of claims 66-70, wherein the agent is associated with the external side of the nanoparticle core.

72. The method of any one of claims 66-71, wherein the agent is encapsulated in the nanoparticle core.

73. The method of any one of claims 66-72, wherein the agent is associated with the oligonucleotide.

74. The method of any one of claims 66-72, wherein the agent is added exogenously.

75. The method of any one of claims 66-74, wherein the agent is a small molecule.

76. The method of claim 75, wherein the small molecule is a dye or a luminophore.

77. The method of claim 76, wherein the dye is a normalizing dye or a dye that localizes to an organelle.

78. The method of any one of claims 66-77, wherein the oligonucleotide is an inhibitory oligonucleotide.

79. The method of claim 78, wherein the inhibitory oligonucleotide is antisense DNA, small interfering RNA (siRNA), an aptamer, a short hairpin RNA (shRNA), a DNAzyme, or an aptazyme.

80. The method of any one of claims 66-79, wherein the oligonucleotide is an immunostimulatory oligonucleotide.

81. The method of claim 80, wherein the immunostimulatory oligonucleotide is a toll-like receptor (TLR) agonist.

82. The method of claim 81, wherein the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof.

83. The method of any one of claims 66-82, wherein the oligonucleotide is a toll-like receptor (TLR) antagonist.

84. The method of claim 83, wherein the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof.

85. The method of any one of claims 66-84, wherein the target analyte is a protein, an ion, a small molecule, a lipid, a carbohydrate, an oligosaccharide, a cell, or a combination thereof.

86. The method of any one of claims 66-84, wherein the target analyte is not a nucleic acid.

87. The method of claim 85, wherein the ion is a metal ion.

88. The method of claim 87, wherein the metal ion is a mercury ion, a copper ion, a silver ion, zinc ion, gold ion, manganese ion, or a combination thereof.

89. The method of claim 85, wherein the ion is a hydrogen ion.

90. The method of claim 89, wherein the detectable change is indicative of a pH change.

91. The method of any one of claims 1-90, wherein the SNA further comprises a therapeutic agent.

92. The method of claim 91, wherein the therapeutic agent is associated with the nanoparticle core.

93. The method of claim 92, wherein the therapeutic agent is encapsulated in the nanoparticle core or is attached to the external side of the nanoparticle core.

94. The method of any one of claims 91-93, wherein the therapeutic agent is associated with the oligonucleotide.

95. The method of any one of claims 1-94, wherein the target analyte is detected intracellularly.
